(19) United States
(12) Patent Application Publication
(10) Pub. No.: US 2025/0327050 A1
(43) Pub. Date: Oct. 23, 2025

(54) ZINC FINGER CCCH-TYPE CONTAINING 14 (ZC3H14) MUTANTS AND METHODS OF USE

(71) Applicants: The Children's Medical Center Corporation, Boston, MA (US); Dana-Farber Cancer Institute, Inc., Boston, MA (US)

(72) Inventors: Leonard I. Zon, Brookline, MA (US); Megan L. Insco, Boston, MA (US)

(73) Assignees: The Children's Medical Center Corporation, Boston, MA (US); Dana-Farber Cancer Institute, Inc., Boston, MA (US)

(21) Appl. No.: 18/291,414

(22) PCT Filed: Jul. 21, 2022
(86) PCT No.: PCT/US2022/037839

§ 371 Date: Jan. 23, 2024

Related U.S. Application Data
(60) Provisional application No. 63/225,174, filed on Jul. 23, 2021.

(51) Int. Cl. C12N 9/22; A61K 38/00; A61P 35/00; C07K 14/47

(52) U.S. Cl. CPC C12N 9/222; A61P 35/00; C07K 14/4702; A61K 38/00; C07K 2319/81

(57) ABSTRACT

The disclosure provides polypeptides, polynucleotides, compositions, kits and methods useful for modulating RNA molecules including degrading disease-causing RNA(s) or stabilizing RNA(s) to treat diseases.

### CROSS-REFERENCE TO RELATED APPLICATIONS

This application is a 35 U.S.C. § 371 National Phase Entry Application of International Application No. PCT/US2022/037839 filed Jul. 21, 2022, which designated the U.S., which claims benefit under 35 U.S.C. § 119(e) of U.S. Provisional Application No. 63/225,174 filed on Jul. 23, 2021, content of which is incorporated herein by reference in its entirety.

### GOVERNMENT SUPPORT

This invention was made with government support under Grant number CA103846, awarded by The National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Oct. 24, 2024, is named 046094-785N01US_SL.xml and is 51,545 bytes in size.

### TECHNICAL FIELD

The technology described herein relates to polypeptides, polynucleotides, compositions, kits and methods that are useful for modulating aberrant RNAs.

### BACKGROUND

The numerous quality control pathways that target defective ribonucleic acids (RNAs) for degradation play key roles in shaping mammalian transcriptomes and preventing disease. These pathways monitor most steps in the biogenesis of both noncoding RNAs (ncRNAs) and protein-coding messenger RNAs (mRNAs), degrading many types of ncRNAs and eliminating mRNAs with a variety of defects that encode abnormal, potentially toxic proteins.

There remains a need in the art for composition and methods for modulating, e.g., degrading disease-causing RNA(s) or stabilizing RNA(s) to alleviate disease. The present disclosure addresses some of these needs.

### SUMMARY

In one aspect provided herein is polypeptide. Generally, the polypeptide comprises at least a first domain comprising an amino acids sequence having at least 80% identity to the amino acid sequence of a wild-type Zinc Finger CCCH-Type Containing 14 (ZC3H14) protein. In some embodiments of any one of the aspects, the amino acid sequence of the first domain comprises a mutation at position 475 of the human ZC3H14 or a homologous or orthologous ZC3H14 protein.

In some embodiments of any one of the aspects, the serine at position 475 of the human ZC3H14 or a homologous or orthologous ZC3H14 protein is replaced with a phosphoserine mimetic. In some other embodiments of any one of the aspects, the serine at position 475 of the human ZC3H14 or a homologous or orthologous ZC3H14 protein is replaced with a moiety that is not a phosphoserine mimetic.

In some embodiments of any one of the aspects, the polypeptide further comprises a moiety for binding with a nucleic acid, e.g., an RNA. For example, the polypeptide is linked to a nucleic acid binding domain of a nucleic acid binding protein. Accordingly, in some embodiments, the polypeptide comprises a first domain comprising an amino acid sequence having at least 85% identity to the amino acid sequence of a wild-type ZC3H14 and a second domain comprising an amino acid sequence having at least 80% identity to a nucleic acid binding domain of a nucleic acid binding protein.

In another aspect, provided herein is a method for degrading or stabilizing an RNA. Generally, the method comprises contacting the RNA with a polypeptide described herein. It is noted that contacting with the RNA can be in a cell. Further, contacting with the RNA can be in vitro or in vivo. When the RNA is in a cell, the polypeptide or a nucleic acid encoding the polypeptide can be administered to the cell.

In yet another aspect, provided herein is a method of treating a disease or disorder characterized by aberrant RNA expression. The method comprises administering a polypeptide described herein or a polynucleotide encoding said polypeptide to a subject in need thereof.

In still another aspect, provided herein is a composition comprising a polypeptide or a nucleic acid encoding the polypeptide described herein.

In yet still another aspect, provided herein is a kit comprising a polypeptide or a nucleic acid encoding the polypeptide described herein.

In still yet another aspect, provided herein is a cell comprising a polypeptide or a nucleic acid encoding the polypeptide described herein.

### BRIEF DESCRIPTION OF THE DRAWINGS

FIG. 1 Model of CDK13's activation of PAXT via ZC3H14 S475 phosphorylation.

FIG. 2A Bar graph showing total peptides as a % of bait (ZC3H14 total peptides) for proteins whose binding was most differentially regulated by CDK13 kinase activity. This was generated from immunoprecipitation (IP) mass spectrometry of ZC3H14. Mean+SD. n=biologic replicates. Gray dots=individual values.

FIGS. 2B and 2C show mass spectrometry chromatogram of immunoprecipitated ZC3H14^{S475} phospho peak (mass 722.69) in CDK13^{WT} cells (FIG. 2B) and CDK13^{R860Q} cells (FIG. 2C). Red peaks=control protein peak. Green peaks=ZC3H14^{S475}

FIG. 2D is a heatmap of average total peptides isolated from ZC3H14 IPs. Data filtered for >3 total peptides in all replicates and >3× enrichment of ZC3H14^{WT} IP in CDK13^{WT} cells as compared to control IP. Row 1=CLOVER (control) IP from CDK13^{WT} cells (n=2) & from CDK13^{mut} (n=2) cells. Row 2=ZC3H14^{WT} IP from CDK13^{WT} cells (n=3). Row 3=ZC3H14^{WT} IP from CDK13^{mut} cells (n=3). Row 4=ZC3H14^{S475D} IP from CDK13^{mut} cells (n=2). Row 5=ZC3H14^{S475A} IP from CDK13^{WT} cells (n=2).

FIG. 2E shows the total number of ZC3H14 peptides detected in each ZC3H14 IP condition and replicate. Box labels the cell line from which ZC3H14 was IPed. One-way ANOVA with multiple comparisons showed amount of protein pulled down in each condition is not significantly different for all comparisons.

FIG. 3A is a box plot showing log 2 fold change of siZFC3H1, ZC3H14^{S475A} A, or ZC3H14^{S475E} as compared to the relevant control for ptRNAs (left), last exons (middle), or internal exons (right). Significantly changed genes (q<0.1) plotted for all categories. Z1=ZFC3H1, Z14=ZC3H14. ptRNA=RNAs generated by usage of intronic polyadenylation sites. Last exon=last exon in a gene. Internal exon=constitutive internal exon (non-alternatively spliced) isoform. The black horizontal line indicates the median and whiskers extend to 1.5× the interquartile range (IQR), unless the maximum is > or the minimum is < that the 1.5× the IQR, in which case they extend to max/min respectively.

FIG. 3B is an IGV plot of TP53 ptRNA which is regulated by CDK13, ZFC3H1, and ZC3H14 S475 phosphorylation.

FIGS. 3C and 3D are schema and immunoblots for samples used in FIG. 3A and FIGS. 3B, 3E, 4A-4C and 5.

FIG. 3E is a boxplot of log 2 fold change of siZC3H14 vs. control significantly changed RNAs (p<0.1). IPA=intronic polyadenylation. Last=last exon. Internal=constitutive internal exon. The black horizontal line indicates the median and whiskers extend to 1.5× the interquartile range (IQR), unless the maximum is > or the minimum is < that the 1.5× the IQR, in which case they extend to max/min respectively.

FIGS. 4A-4C show mean average plot of ptRNA isoforms from RNA-seq from A375 human melanoma cells with genetic changes affecting nuclear RNA surveillance vs. Control. Specifically, (FIG. 4A) ZC3H14 S475E vs. Control (CLOVER fluorescent protein) (FIG. 4B) ZC3H14 S475A vs. Control (CLOVER fluorescent protein) and (FIG. 4C) knockdown of PAXT key adaptor ZFC3H1 as compared to control knockdown.

FIG. 5 shows mean average plot of detained intron isoforms from RNA-seq from A375 human melanoma cells with expression of ZC3H14 S475E vs. Control (CLOVER fluorescent protein).

FIG. 6A shows models for how digital droplet PCR of individual targets can be used to discern the mechanism of ptRNA accumulation in CDK13^{mut} cells.

FIG. 6B shows RNA expression in CDK13^{mut}/control human melanoma cells measured by digital droplet PCR for 4 genes with increased ptRNAs by 3′ sequencing and 2 control genes. F=first exon. L=last exon. 2-way ANOVA, multiple comparisons. Adjusted p-values from left to right=****<0.0001, ***=0.0006, *=0.0280, ***=0.0004, ns=0.8626, ns=0.9158.

FIG. 7 shows siRNA knockdown of ZFC3H1 causes accumulation of RNAs with detained introns.

FIGS. 8A and 8B show CDK13/CCNT1 directly phosphorylates full length ZC3H14^{WT} on the 475 residue. (FIG. 8A) in vitro kinase assay P32 counts per minute minus time zero shows that CDK13 directly phosphorylates ZC3H14 WT but not ZC3H14 S475A. (FIG. 8B) anti-thiophosphorylation immunoblot shows that CDK13 directly phosphorylates ZC3H14 WT but not ZC3H14 S475A.

FIGS. 9A and 9B show CDK13 mutant oncogenesis is reversed by expression of an exemplary polypeptide (ZC3H14 S475E) according to an embodiment of the disclosure. (FIG. 9A) Western shows expression of ZC3H14 and control CLOVER in CDK13 mutant human melanoma A375 cells. (FIG. 9B) Cell counts from the lines in FIG. 9A show that ZC3H14^{S475E} is sufficient to reverse CDK13 mutant increased proliferation.

FIGS. 10A-10D show expression of human ptRNAs is sufficient to cause oncogenesis in a zebrafish BRAFV600E p53−/− model zebrafish. (FIGS. 10A and 10B) Triples melanoma model zebrafish with melanocyte-specific expression of EGFP or human TP53 ptRNA. (FIG. 10A) 7-week photos. (FIG. 10B) % melanoma-free survival (log-rank). (#)=zebrafish. (FIGS. 10C and 10D) Triples melanoma model zebrafish with melanocyte-specific expression of EGFP or human SUV39H1 ptRNA. (FIG. 10C) 7-week photos. (FIG. 10D) % melanoma-free survival (log-rank). (#)=zebrafish.

### DETAILED DESCRIPTION

Various aspects described herein include a polypeptide comprising at least a first domain, wherein the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to the amino acid sequence of a wild-type ZC3H14. For example, the first domain comprises an amino acid sequence having at least 85%, 86%, 87%, 88%, 89%, 90%, 91%, 92%, 93%, 94%, 95%, 96%, 97%, 98% or 99% identity to the amino acid sequence of a wild-type ZC3H14. In some embodiments, the first domain comprises an amino acid sequence having at least 90%, 91%, 92%, 93%, 94%, 95%, 96%, 97%, 98% or 99% identity to the amino acid sequence of a wild-type ZC3H14. In some preferred embodiments, the first domain comprises an amino acid sequence having at least 95%, 96%, 97%, 98% or 99% identity to the amino acid sequence of a wild-type ZC3H14.

ZC3H14 is a zinc-finger protein that has been shown to bind poly A tails (Fasken et al., 2019; Morris and Corbett, 2018) and the PAXT complex (Meola et al., 2016). Inventors have shown for the first time that ZC3H14 regulates the PAXT complex. Specifically, they found ZC3H14 S475 phosphorylation as necessary and sufficient for PAXT binding and activation. Exemplary sequences for wild-type ZC3H14 can be found in Genebank with Accession Numbers as listed in Table 1.

TABLE 1
Accession Numbers for exemplary wild-type
ZC3H14 amino acid sequences

| Species | Accession No. |
|---|---|
| Homo sapiens | NP_079100.2 |
| Mus musculus | NP_083610.2 |
| Rattus norvegicus | NP_001029123.1 |
| Gallus gallus | NP_001012604.1 |
| Pan troglodytes | XP_009426555.1 |
| Canis lupus familiaris | XP_854349.2 |
| Bos taurus | NP_001159774.1 |
| Danio rerio | NP_001020695.2 |
| Macaca mulatta | NP_001253521.1 |
| Monodelphis domestica | XP_016278461.1 |
| Equus caballus | XP_023484005.1 |
| Ornithorhynchus anatinus | XP_028915497.1 |
| Sus scrofa | XP_005666426.1 |
| Pongo abelii | XP_009247642.1 |
| Taeniopygia guttata | XP_012429743.4 |
| Oryctolagus cuniculus | XP_008270165.1 |
| Callithrix jacchus | XP_035117140.1 |
| Ailuropoda melanoleuca | XP_011224153.1 |
| Xenopus tropicalis | XP_004917117.2 |
| Meleagris gallopavo | XP_010710108.1 |
| Anolis carolinensis | XP_008114279.1 |
| Nomascus leucogenys | XP_030659779.1 |
| Loxodonta africana | XP_010587099.1 |
| Oreochromis niloticus | XP_005460334.1 |
| Cavia porcellus | XP_005004918.1 |
| Cricetulus griseus | XP_007640237.1 |
| Sarcophilus harrisii | XP_012402866.3 |
| Otolemur garnettii | XP_003787017.1 |
| Pan paniscus | XP_003832794.1 |
| Papio anubis | XP_003902190.1 |
| Saimiri boliviensis | XP_039324731.1 |
| Takifugu rubripes | XP_011610354.2 |
| Felis catus | XP_023111703.1 |
| Ovis aries | XP_027827904.1 |
| Gorilla gorilla | XP_018865411.1 |
| Oryzias latipes | XP_020570309.1 |
| Orcinus orca | XP_012387649.1 |
| Tursiops truncatus | XP_019774823.1 |
| Odobenus rosmarus divergens | XP_004398154.1 |
| Dasypus novemcinctus | XP_004470372.1 |
| Maylandia zebra | XP_004541935.1 |
| Ochotona princeps | XP_036352239.1 |
| Sorex araneus | XP_012788584.1 |
| Octodon degus | XP_004635171.1 |
| Jaculus jaculus | XP_004649405.1 |
| Condylura cristata | XP_004681694.1 |
| Echinops telfairi | XP_012859134.1 |
| Mustela putorius furo | XP_004739147.2 |
| Heterocephalus glaber | XP_004837143.1 |
| Anas platyrhynchos | XP_038035323.1 |
| Ficedula albicollis | XP_005047505.1 |
| Mesocricetus auratus | XP_005068475.1 |
| Macaca fascicularis | XP_005562047.1 |
| Falco peregrinus | XP_005241966.1 |
| Chrysemys picta | XP_005285431.1 |
| Ictidomys tridecemlineatus | XP_005321362.1 |
| Microtus ochrogaster | XP_005343490.1 |
| Chinchilla lanigera | XP_005390315.2 |
| Geospiza fortis | XP_030920918.1 |
| Zonotrichia albicollis | XP_005483204.1 |
| Columba livia | XP_021148188.1 |
| Pseudopodoces humilis | XP_014105516.1 |
| Capra hircus | XP_017909360.1 |
| Pundamilia nyererei | XP_005734412.1 |
| Xiphophorus maculatus | XP_023202962.1 |
| Myotis brandtii | XP_005871228.1 |
| Bos mutus | XP_005911963.1 |
| Haplochromis burtoni | XP_005914480.1 |
| Latimeria chalumnae | XP_005986584.1 |
| Alligator sinensis | XP_025071338.1 |
| Bubalus bubalis | XP_025150907.1 |
| Myotis lucifugus | XP_023608286.1 |
| Pelodiscus sinensis | XP_006131737.1 |
| Tupaia chinensis | XP_027631296.1 |
| Camelus ferus | XP_032338372.1 |
| Vicugna pacos | XP_006208239.1 |
| Alligator mississippiensis | XP_006268082.1 |
| Lepisosteus oculatus | XP_015205778.1 |
| Leptonychotes weddellii | XP_006742262.1 |
| Myotis davidii | XP_006754796.1 |
| Chrysochloris asiatica | XP_006839648.1 |
| Elephantulus edwardii | XP_006879279.1 |
| Pteropus alecto | XP_006925572.1 |
| Peromyscus maniculatus bairdii | XP_015860902.1 |
| Chelonia mydas | XP_037756739.1 |
| Panthera tigris altaica | XP_007095048.1 |
| Physeter catodon | XP_023986704.1 |
| Balaenoptera acutorostrata | XP_007188782.1 |
| scammoni | |
| Astyanax mexicanus | XP_007259926.2 |
| Python bivittatus | XP_007431647.1 |
| Lipotes vexillifer | XP_007462794.1 |
| Erinaceus europaeus | XP_016045375.1 |
| Poecilia formosa | XP_007552970.1 |
| Callorhinchus milii | XP_007902012.1 |
| Chlorocebus sabaeus | XP_007985724.1 |
| Eptesicus fuscus | XP_008146627.1 |
| Stegastes partitus | XP_008288609.1 |
| Cynoglossus semilaevis | XP_024909497.1 |
| Poecilia reticulata | XP_008396106.1 |
| Calypte anna | XP_030308220.1 |
| Equus przewalskii | XP_008534621.1 |
| Corvus brachyrhynchos | XP_008627917.1 |
| Ursus maritimus | XP_040497497.1 |
| Nannospalax galili | XP_008842014.1 |
| Merops nubicus | XP_008936656.1 |
| Acanthisitta chloris | XP_009068343.1 |
| Serinus canaria | XP_009100073.1 |
| Aptenodytes forsteri | XP_009279036.1 |
| Pygoscelis adeliae | XP_009317706.1 |
| Nipponia nippon | XP_009474200.1 |
| Pelecanus crispus | XP_009487830.1 |
| Cuculus canorus | XP_009559600.1 |
| Egretta garzetta | XP_009637151.1 |
| Cariama cristata | XP_009694692.1 |
| Gavia stellata | XP_009818665.1 |
| Apaloderma vittatum | XP_009871626.1 |
| Charadrius vociferus | XP_009882853.1 |
| Dryobates pubescens | XP_009903136.1 |
| Haliaeetus albicilla | XP_009912774.1 |
| Opisthocomus hoazin | XP_009935159.1 |
| Leptosomus discolor | XP_009957261.1 |
| Tyto alba | XP_032866441.1 |
| Tauraco erythrolophus | XP_009984777.1 |
| Chaetura pelagica | XP_010002006.1 |
| Nestor notabilis | XP_010009120.1 |
| Pterocles gutturalis | XP_010085392.1 |
| Buceros rhinoceros silvestris | XP_010141934.1 |
| Eurypyga helias | XP_010149583.1 |
| Antrostomus carolinensis | XP_028942884.1 |
| Mesitornis unicolor | XP_010180397.1 |
| Colius striatus | XP_010208804.1 |
| Tinamus guttatus | XP_010223270.1 |
| Phaethon lepturus | XP_010287300.1 |
| Balearica regulorum gibbericeps | XP_010302453.1 |
| Rhinopithecus roxellana | XP_010382421.1 |
| Haliaeetus leucocephalus | XP_010582103.1 |
| Larimichthys crocea | XP_027140397.1 |
| Bison bison bison | XP_010843455.1 |
| Esox lucius | XP_028970513.1 |
| Camelus bactrianus | XP_010966976.1 |
| Camelus dromedarius | XP_031310021.1 |
| Pteropus vampyrus | XP_011355426.1 |
| Macaca nemestrina | XP_011715593.1 |
| Mandrillus leucophaeus | XP_011829681.1 |
| Cercocebus atys | XP_011939023.1 |
| Aotus nancymaae | XP_021530631.1 |
| Microcebus murinus | XP_020141170.1 |
| Clupea harengus | XP_031438003.1 |
| Fundulus heteroclitus | XP_021168184.2 |
| Dipodomys ordii | XP_012871508.1 |
| Apteryx mantelli mantelli | XP_013796093.1 |
| Thamnophis sirtalis | XP_013931584.1 |
| Equus asinus | XP_014701347.1 |
| Sturnus vulgaris | XP_014725348.1 |
| Calidris pugnax | XP_014802515.1 |
| Poecilia mexicana | XP_014846249.1 |
| Poecilia latipinna | XP_014876399.1 |
| Acinonyx jubatus | XP_026922177.1 |
| Cyprinodon variegatus | XP_015229002.1 |
| Gekko japonicus | XP_015282194.1 |
| Marmota marmota marmota | XP_015350101.1 |
| Parus major | XP_033370784.1 |
| Protobothrops mucrosquamatus | XP_015680971.1 |
| Coturnix japonica | XP_015720780.1 |
| Nothobranchius furzeri | XP_015802642.1 |
| Rousettus aegyptiacus | XP_015979280.1 |
| Miniopterus natalensis | XP_016065590.1 |
| Sinocyclocheilus rhinocerous | XP_016382243.1 |
| Kryptolebias marmoratus | XP_017282851.1 |
| Ictalurus punctatus | XP_017306735.1 |
| Manis javanica | XP_017535359.1 |
| Pygocentrus nattereri | XP_017562499.1 |
| Rhinopithecus bieti | XP_017750854.1 |
| Nanorana parkeri | XP_018418854.1 |
| Lates calcarifer | XP_018549979.1 |
| Scleropages formosus | XP_018611789.1 |
| Panthera pardus | XP_019317522.1 |
| Gavialis gangeticus | XP_019374228.1 |
| Crocodylus porosus | XP_019407929.1 |
| Hipposideros armiger | XP_019497850.1 |
| Hippocampus comes | XP_019740605.1 |
| Bos indicus | XP_019824923.1 |
| Paralichthys olivaceus | XP_019953352.1 |
| Oncorhynchus kisutch | XP_031656407.1 |
| Rhincodon typus | XP_020389292.1 |
| Monopterus albus | XP_020472987.1 |
| Labrus bergylta | XP_020494478.1 |
| Pogona vitticeps | XP_020667075.1 |
| Odocoileus virginianus texanus | XP_020746237.1 |
| Boleophthalmus pectinirostris | XP_020775399.1 |
| Phascolarctos cinereus | XP_020856032.1 |
| Mus caroli | XP_021034289.1 |
| Mus pahari | XP_021058432.1 |
| Numida meleagris | XP_021258420.1 |
| Lonchura striata domestica | XP_021396641.1 |
| Oncorhynchus mykiss | XP_036832251.1 |
| Meriones unguiculatus | XP_021519644.1 |
| Neomonachus schauinslandi | XP_021535481.1 |
| Acanthochromis polyacanthus | XP_022051589.1 |
| Delphinapterus leucas | XP_022432039.1 |
| Seriola dumerili | XP_022622822.1 |
| Amphiprion ocellaris | XP_023152756.1 |
| Cyanistes caeruleus | XP_023783496.1 |
| Oryzias melastigma | XP_024146939.1 |
| Oncorhynchus tshawytscha | XP_024262021.1 |
| Desmodus rotundus | XP_024424157.1 |
| Theropithecus gelada | XP_025247074.1 |
| Canis lupus dingo | XP_025299401.2 |
| Callorhinus ursinus | XP_025725279.1 |
| Puma concolor | XP_025775159.1 |
| Vulpes vulpes | XP_025868306.1 |
| Nothoprocta perdicaria | XP_025900476.1 |
| Apteryx rowi | XP_025935318.1 |
| Dromaius novaehollandiae | XP_025968134.1 |
| Astatotilapia calliptera | XP_025998769.1 |
| Mastacembelus armatus | XP_026175177.1 |
| Anabas testudineus | XP_026197846.1 |
| Urocitellus parryii | XP_026256416.1 |
| Ursus arctos horribilis | XP_026371271.1 |
| Notechis scutatus | XP_026534946.1 |
| Pseudonaja textilis | XP_026568627.1 |
| Athene cunicularia | XP_026705494.1 |
| Electrophorus electricus | XP_026885925.2 |
| Lagenorhynchus obliquidens | XP_026976060.1 |
| Bos indicus x Bos taurus | XP_027408484.1 |
| Zalophus californianus | XP_027428220.1 |
| Neopelma chrysocephalum | XP_027529007.1 |
| Vombatus ursinus | XP_027700668.1 |
| Empidonax traillii | XP_027754944.1 |
| Marmota flaviventris | XP_027787365.1 |
| Xiphophorus couchianus | XP_027896649.1 |
| Eumetopias jubatus | XP_027953852.1 |
| Parambassis ranga | XP_028252894.1 |
| Phyllostomus discolor | XP_035868849.1 |
| Perca flavescens | XP_028460744.1 |
| Podarcis muralis | XP_028573832.1 |
| Erpetoichthys calabaricus | XP_028677296.1 |
| Peromyscus leucopus | XP_028742419.1 |
| Denticeps clupeoides | XP_028821190.1 |
| Betta splendens | XP_028996856.1 |
| Monodon monoceros | XP_029093154.1 |
| Cottoperca gobio | XP_029281037.1 |
| Echeneis naucrates | XP_029352036.1 |
| Rhinatrema bivittatum | XP_029453613.1 |
| Oncorhynchus nerka | XP_029503580.1 |
| Salmo trutta | XP_029550434.1 |
| Suricata suricatta | XP_029807624.1 |
| Aquila chrysaetos chrysaetos | XP_029886067.1 |
| Salarias fasciatus | XP_029965757.1 |
| Microcaecilia unicolor | XP_030069603.1 |
| Lynx canadensis | XP_030175610.1 |
| Gadus morhua | XP_030200758.1 |
| Sparus aurata | XP_030262162.1 |
| Archocentrus centrarchus | XP_030577899.1 |
| Chanos chanos | XP_030637449.1 |
| Globicephala melas | XP_030739489.1 |
| Camarhynchus parvulus | XP_030805398.1 |
| Sander lucioperca | XP_031178178.1 |
| Mastomys coucha | XP_031211156.1 |
| Phasianus colchicus | XP_031448976.1 |
| Oreochromis aureus | XP_031587844.2 |
| Hylobates moloch | XP_031990623.1 |
| Aythya fuligula | XP_032045072.1 |
| Thamnophis elegans | XP_032088638.1 |
| Sapajus apella | XP_032143567.1 |
| Mustela erminea | XP_032200627.1 |
| Phoca vitulina | XP_032261635.1 |
| Etheostoma spectabile | XP_032398443.1 |
| Xiphophorus hellerii | XP_032441897.1 |
| Phocoena sinus | XP_032477182.1 |
| Chiroxiphia lanceolata | XP_032547361.1 |
| Chelonoidis abingdonii | XP_032630099.1 |
| Lontra canadensis | XP_032734477.1 |
| Rattus rattus | XP_032764138.1 |
| Catharus ustulatus | XP_032919137.1 |
| Rhinolophus ferrumequinum | XP_032965096.1 |
| Lacerta agilis | XP_032997418.1 |
| Trachypithecus francoisi | XP_033047550.1 |
| Geotrypetes seraphini | XP_033807385.1 |
| Pantherophis guttatus | XP_034274181.1 |
| Arvicanthis niloticus | XP_034344129.1 |
| Trachemys scripta elegans | XP_034624021.1 |
| Mirounga leonina | XP_034848413.1 |
| Zootoca vivipara | XP_034963475.1 |
| Oxyura jamaicensis | XP_035184832.1 |
| Cygnus atratus | XP_035395199.1 |
| Halichoerus grypus | XP_035967386.1 |
| Onychomys torridus | XP_036061619.1 |
| Molossus molossus | XP_036118528.1 |
| Myotis myotis | XP_036170868.1 |
| Molothrus ater | XP_036240246.1 |
| Pipistrellus kuhlii | XP_036285352.1 |
| Trichosurus vulpecula | XP_036590511.1 |
| Balaenoptera musculus | XP_036700912.1 |
| Manis pentadactyla | XP_036738276.1 |
| Sturnira hondurensis | XP_036887504.1 |
| Artibeus jamaicensis | XP_036986135.1 |
| Falco rusticolus | XP_037249667.1 |
| Talpa occidentalis | XP_037359630.1 |

In some embodiments of any one of the aspects, the amino acid sequence of the first domain comprises a mutation at position 475 of the human ZC3H14 or a homologous or orthologous ZC3H14 protein amino acid sequence. For example, the first domain comprises an amino acid sequence having a phosphoserine mimetic at position 475 of the human ZC3H14 or a corresponding position in a homologous or orthologous ZC3H14 protein. As used herein, a “phosphoserine mimetic” is a moiety that appears chemically or functionally similar to a phosphorylated serine. In other words, a phosphoserine mimetic is a moiety that can be substituted for a phosphoserine. It is noted that the phosphoserine mimetic can be an amino acid, e.g., a phosphoserine mimetic amino acid, or a non-amino acid moiety, e.g., a non-hydrolyzable phosphoserine mimetic. Some exemplary phosphoserine mimetic amino acid amino acids include, but are not limited to, aspartic acid, glutamic acid and phosphothreonine.

The chemical structure of a non-amino acid phosphoserine mimetic can in some embodiments closely approximate the natural phosphoserine, and also be chemically stable (e.g., resistant to dephosphorylation by phosphatase enzymes). This can be achieved with a synthetic molecule in which the phosphorous atom is linked to the amino acid residue, not through oxygen, but through carbon. In some embodiments, a CF₂ group links the amino acid to the phosphorous atom. Alternatively, the oxygen bridge of the natural amino acid can be replaced with a methylene group. One exemplary non-hydrolyzable phosphoserine mimetic is L-2-amino-4 (diethylphosphono)-4,4-difluorobutanoic acid.

In some embodiments of any one of the aspects, the first domain comprises an amino acid sequence having a non-phosphoserine mimetic at position 475 of the human ZC3H14 or a corresponding position in a homologous or orthologous ZC3H14 protein. As used herein, a “non-phosphoserine mimetic” is a moiety that is chemically and functionally different from phosphoserine. In other words, a non-phosphoserine mimetic is a moiety that cannot function as a substitute for phosphoserine. The non-phosphoserine mimetic can be an amino acid or a non-amino acid moiety. For example, the non-phosphoserine mimetic can be a conservative variant of serine. In some embodiments, the non-phosphoserine mimetic can be a residue that cannot be phosphorylated, i.e., is non-phosphorylatable. In some embodiments, the non-phosphoserine mimetic can be an amino acid selected from the group consisting of alanine, valine, leucine, isoleucine, methionine, phenylalanine, tryptophan, cysteine, glycine, proline, and selenocystine. In some preferred embodiments, the non-phosphoserine mimetic is alanine.

In some embodiments, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the group consisting of:

(SEQ ID NO: 1, human ZC3H14,)
MEIGTEISRKIRSAIKGKLQELGAYVDEELPDYIMVMVANKKSQDQMTEDLSLFLGNNTI
RFTVWLHGVLDKLRSVTTEPSSLKSSDTNIFDSNVPSNKSNFSRGDERRHEAAVPPLAIP
SARPEKRDSRVSTSSQESKTTNVRQTYDDGAATRLMSTVKPLREPAPSEDVIDIKPEPDD
LIDEDLNFVQENPLSQKKPTVTLTYGSSRPSIEIYRPPASRNADSGVHLNRLQFQQQQNS
IHAAKQLDMQSSWVYETGRLCEPEVLNSLEETYSPFFRNNSEKMSMEDENFRKRKLPVVS
SVVKVKKFNHDGEEEEEDDDYGSRTGSISSSVSVPAKPERRPSLPPSKQANKNLILKAIS
EAQESVTKTTNYSTVPQKQTLPVAPRTRTSQEELLAEVVQGQSRTPRISPPIKEEETKGD
SVEKNQGTQQRQLLSRLQIDPVMAETLQMSQDYYDMESMVHADTRSFILKKPKLSEEVVV
APNQESGMKTADSLRVLSGHLMQTRDLVQPDKPASPKFIVTLDGVPSPPGYMSDQEEDMC
FEGMKPVNQTAASNKGLRGLLHPQQLHLLSRQLEDPNGSFSNAEMSELSVAQKPEKLLER
CKYWPACKNGDECAYHHPISPCKAFPNCKFAEKCLFVHPNCKYDAKCTKPDCPFTHVSRR
IPVLSPKPAVAPPAPPSSSQLCRYFPACKKMECPFYHPKHCRENTQCTRPDCTFYHPTIN
VPPRHALKWIRPQTSE;
(SEQ ID NO: 2, Mus musculus)
MEIGTEISRKIRSAIKGKLQELGAYVDEELPDYIMVMVANKKSQDQMTEDLSLFLGNNTI
RFTVWLHGVLDKLRSVTTEPSSLKSPDASIFDSHVPSNKSSFSRGDERRHEAAVPPLAVS
SSRPEKRDSRVSTSSQEQKSTNVRHSYDDGASTRLMSTVKPLREPAPSEDVIDIKPEPDD
LIDEDLNFVQENPLSQKKPTVTLTYGSSRPSIEIYRPPASRNADTGTHLNRLQLHPQQSS
AHAAKQLDVQSSQVSEAGRLCEPPVLSSVEDTYSPFFRNNLDKMSIEDENFRKRKLPVVS
SVVKVKRFSHDGEEEEEDEDYGTRIGSLSSSVSVPAKPERRPSLPPSKQANKNLILKAIS
EAQESVTKTTNYSAVPQKQTLPVAPRTRTSQEELLAEMVQGQNRAPRISPPVKEEEAKGD
NTGKSQGTQQRQLLSRLQIDPVMVETMEMSQDYYDMESMVHADTRSFILKKPKLSEEIVV
TPNQDSGMKTADALRVLSGHLMQTRDLVQPDKPASPKFIVTLDGVPSPPGYMSDQEEEMC
FEGMKPVNQTSASNKGLRGLLHPQQLHLLSRQLEDPDGSESNAEMTDLSVAQKPEKLLER
CKYWPACKNGDECVYHHPISPCKAFPNCKFAEKCLFVHPNCKYDTKCTKADCPFTHMSRR
ASILTPKPVSSPAPSSNGQLCRYFPACKKMECPFYHPKHCRENTQCTRPDCTFYHPTITV
PPRHALKWIRPQSSE;
(SEQ ID NO: 3, rattus norvegicus)
MEIGTEISRKIRSAIKGKLQELGAYVDEELPDYIMVMVANKKSQDQMTEDLSLFLGNNTI
RFTVWLHGVLDKLRSVTTEPSSLKSPDTSIFDSNVPSNKSSFSRGDERRHEAAIPPLAVS
SSRPEKRDSRVSTSSQEHKSTNVRHSYDDGASTRLMSTVKPLREPAPSEDVIDIKPEPDD
LIDEDLNFVQENSLSQKKPTVTLTYGSSRPSIEIYRPPASRNADTGTHLNRPQLQQQQSS
THTAKQLDGQSSQVYEAGRLCEPEVLGSVEDTYSPFFRNNLDKMNIEEENFRKRKLPVVS
SVVKVKRFSHDGEEEEEDEDYGTRVGSLSSSVSVPAKPERRPSLPPSKQANKNLILKAIS
EAQESVTKTTNYPAVPQKQTLPVAPRTRTSQEEVLAEMVQGQNRAPRISPPVKEEEAKGD
NAEKIEGTQQRQLLSRLQIDPVTVDTMELSQDYYDMESMVHADTRSFILKKPKLSEEIVV
TPNQDSGMKTADALRVLSGHLMQTRDLVQPDKPASPKFIVTLDGVPSPPGYMSDQEEEMC
FEGMKPVNQTSASNKGLRGLLHPQQLHLLSRQLEDPDGSFSNAEMTDLSVAQKPEKLLER
CKYWPACKNGDECVYHHPISPCKAFPNCKFAEKCLFVHPNCKYDAKCTKADCPFTHMSRR
GPVLTPKPAVSSPAPSSNGQFCRYFPACKKMECPFYHPKHCRENTQCTRPDCTFYHPTIT
VPPRHALKWIRPQTSE;
(SEQ ID NO: 4, Gallus gallus)
MEIGTEISRKIRGAIKGKLQELGAYVDEELPDYIMVMVANKKSQEQMTEDLSLFLGNNTV
RFTVWLHGVLDKLRSVTTEPSGTKSSEPNIFESNHSSSKSSSCVSDERRREDTLPPLAVS
STRSERTDSRVSTSSQEQRNTASRQSCEDGSASRLMSTVKPLRELSPSEAVIDIKPEPDD
LIDEDLNFVQENPLSRKKPIVTVTYGSSRPTAEIYRPPASRSADGSLQVHRLPQQGNLQG
NRQLDTQSCRSLETVQLCNPEAFGSLAESYRPTSKLSADKVGSEEEGSRKRRLPIVSSVV
KVKKFCNDGEEEEEEDDYGLRTGSISSSVSVPAKPERRPSLPPSKQVNKNLILKAISEAQ
ESVTKTTNYSAVPQKQTVPVAPRTRISPEESHLEVIHLQSRLPALCSQLQVEEPKEQAVE
GIQGAEQKELSSRLQVDPVIEDTLQVTQDYYDGESMVHTDTRSFILKKPKLSEEIAAQNQ
QLGKRATEAMRVLSGRLIQTRDQIAQPEKPASPKFIVTLDGVPSPPGYLSDQEEEDMYIT
EGLKPIPQNICAGKGLKGLRAQQMQIVTRQLDSSDVEMEQLNVLQKQEKVLERCKYWPAC
KNGDECVYHHPTQPCKVFPNCKFADKCLFIHPNCKYDAKCTKPDCPYTHASRRNPLPSPK
PVPLPTQSVSSSSPLCKFFPACKKMECPFYHPKHCRFNTQCTRPDCTFYHPTIAVPPRHA
LKWTRTQTSE;
(SEQ ID NO: 5, chimpanzee)
MEIGTEISRKIRSAIKGKLQELGAYVDEELPDYIMVMVANKKSQDQMTEDLSLFLGNNTI
RFTVWLHGVLDKLRSVTTEPSSLKSSDTNIFDSNVPSNKSNFSRGDERRHEAAVPPLAIP
SARPEKRDSRVSTSSQESKTTNVRQTYDDGAATRLMSTVKPLREPAPSEDVIDIKPEPDD
LIDEDLNFVQENPLSQKKPTVTLTYGSSRPSIEIYRPPASRNADSGVHLNRLQFQQQQNS
IHAAKQLDMQSSWVYETGRLCEPEVLNSLEETYSPFFRNNSEKMSMEDENFRKRKLPVVS
SVVKVKKFNHDGEEEEEDDDYGSRTGSISSSVSVPAKPERRPSLPPSKQANKNLILKAIS
EAQESVTKTTNYSTVPQKQTLPVAPRTRTSQEELLAEVVQGQSRTPRISPPIKEEETKGD
SVEKNQGTQQRQLLSRLQIDPVMAETLQMSQDYYDMESMVHADTRSFILKKPKLSEEIVV
APNQESGMKTADSLRVLSGHLMQTRDLVQPDKPASPKFIVTLDGVPSPPGYMSDQEEDMC
FEGMKPVNQTAASNKGLRGLLHPQQLHLLSRQLEDPNGSFSNAEMSELSVAQKPEKLLER
CKYWPACKNGDECAYHHPISPCKAFPNCKFAEKCLFVHPNCKYDAKCTKPDCPFTHVSRR
IPVLSPKPAVAPPAPPSSSQLCRYFPACKKMECPFYHPKHCRFNTQCTRPDCTFYHPTIN
VPPRHALKWIRPQTSE;
(SEQ ID NO: 6, Canis lupus familiaris)
MEIGTEISRKIRSAIKGKLQELGAYVDEELPDYIMVMVANKKSQDQMTEDLSLFLGNNTI
RFTVWLHGVLDKLRSVTTDPSSLKSSDTNIFDNNVSSNKSSFSRGDERRHEAAVPPLAVA
STRPEKRDSRVSTSSQEQKTTNVRQTYDDGAATRLMSTVKPLREPAPSEDVIDIKPEPDD
LIDEDLNFVQENPLSQKKPTVTLTYGSSRPSIEIYRPPASRNADSGAHLNRLQFQQQQNS
IHAAKQPDIQNSRVYETGRLCEPEVLNSLEDTYSPFFRNNSEKMSIEEENFRKRKLPVVS
SVVKVKKFNHDGEEEEEDDDCGSRTGSISSSVSVPAKPERRPSLPPSKQANKNLILKAIS
EAQESVTKTTNYSTVSQKQTLPVAPRTRTSQEELLAEMVQGQSRTPRISPPMKEEETKGD
NIEKSQGTQQRQLLSRLQIDPVMAETLQISQDYYDMESMVHADTRSFILKKPKLSEEIVV
ASNQESGMKTADTLRVLSGHLMQTRDLVQPDKPASPKFIVTLDGVPSPPGYMSDQEEDMC
FEGVKPVNQTAASNKGLRGLLHPQQLQLMSRQLDDPNGSFLNAEMSELSVVQKPEKLLER
CKYWPACKNGDECAYHHPVSPCKAFPNCKFAEKCLFVHPNCKYDAKCTKPDCPFTHMSRR
IPVLPPKPAIATPAPPSSSQLCRYFPACKKMECPFYHPKHCRENTQCTRPDCTFYHPTIT
VPPRHALKWIRPQTSE;
(SEQ ID NO: 7, Bos taurus)
MEIGTEISRKIRSAIKGKLQELGAYVDEELPDYIMVMVANKKSQDQMTEDLSLFLGNNTI
RFTVWLHGVLDKLRSVTTDPASLKSSDTNLFDGNVPSNKSSFSRGDERRHEAAVPPLAVS
STRPEKRESRVSTSSQEQKATNVRQTYDDGAATRLMSTVKPLRELAPSEDVIDIKPEPDD
LIDEDLNFVQENPLSQKKTTVTLTYGSSRPSIEIYRPPATRNTDSGAHLNRLQFQQQQNS
IHAAKQLDIQSSRVYETGRLCEPEVLNSLEETYSPFFRSNAEKMSIEEENFRKRKLPVVS
SVVKVKKFSHDGEEEEEDDDCGSRTGSISSSVSVPAKPERRPSLPPSKQANKNLILKAIS
EAQESVTKTTNYSTVSQKQTLPVAPRTRTSQEDLLAEVAQGHGRVPRISSPVKEEEAQGG
SVDERQGTQQRQLLSRLQIDPVMAETLQISQDYYDMESMVHADTRSFILKKPKLCEELVV
AASQASGMETADALRARSGHLVQTRDLVQPDKPASPKFIVTLDGVPSPPGYMSDQEEDMC
SEGMRPAQHPAASHGGLAGLLHPQRSRVLSRQLEDPDGSFANAEMSELSVAQKPEKLLER
CKYWPACKNGDECAYHHPVSPCKAFPNCKFAEKCLFVHPNCKYDAKCTKPDCPFTHMSRR
TPGLPPKPVTAPAPPSSSQLCRYFPACKKMECPFYHPKHCRFNTQCTRPDCAFYHPTITV
PPRHALKWIRPQTSD;
(SEQ ID NO: 8, Danio rerio)
MEIGTEISKKIRTAIKRKLQEFGAYVDEELPDYIMVMVANKKNPQQMADDLSLFLGNNTI
KFTVWLHGVLEKLRSVAVEPPSLGPSVVHSETSIPAENSRRGAEPRAIAVSSSRSDKAEG
RVSSSAHENRASKRGSSERPSRLTSAVKPLMEASAEAVIDIKPDLDDDLISYDPVEHSLT
SGHSQALYSRSTAERQRPAVESSRQTADTYRSSDISRGQDRSERGYRSSAESSRDLSRKR
KAPVASSVVRVHRGHERGLEIEDLEEEEEDEDYGLASKVSLPSKPERKPTLPPAKQANKN
LILKAISEAQESINKTTSQYTVPQRQTVPVAPRTRSASDEMSNAAIRLVQEHLHALTPQD
TLHNTQSRGLASRLQLEVPEEDSREPHEYELQVLEAARLKALDTRSFIMRQPEVEQPPPI
RSRLSAVNQNENAPTTSRMVQARERAEAVGGSSPKFIVTLDGVPSPLANRTDQEMETEDE
LNTTADLPENNNNNNTTTTTSKPAIHLRLGADERNACDDEVEEMDVEAVQAKRQKLPERC
KFWPTCKSGDECLYHHPNTQCKVFPNCKFADKCLFIHPNCKFDAKCTKADSPFTHVSRRL
NSNPTRAAPALTSTVCRFFPGCKKVDCPFYHPKPCRFATQCKRADCTFYHPAVAVPPRSA
LKWTKTQSS;
(SEQ ID NO: 9, Macaca mulatta)
MEIGTEISRKIRSAIKGKLQELGAYVDEELPDYIMVMVANKKSQDQMTEDLSLFLGNNTI
RFTVWLHGVLDKLRSVTTEPSSLKSSDTNIFDSNVPSNKSNFSRGDERRHEAAVPPLAIP
STRPEKRDSRVSTSSQESKTTNVRQTYDDGAATRLMSTVKPLREPAPSEDVIDIKPEPDD
LIDEDLNFVQENPLSQKKPTVTLTYGSSRPSIEIYRPPASRNADSGVHLNRLQFQQQQNS
IHAAKQLDMQNSWVYETGRLCEPEVLNSLEETYSPFFRNNSEKMSMEDENFRKRKLPVVS
SVVKVKKFNHDGEEEEEDDDYGSRTGSISSSVSVPAKPERRPSLPPSKQANKNLILKAIS
EAQESVTKTTNYSTVPQKQTLPVAPRTRTSQEELLAEVVQGQSRTPRISPPIKEEETKGD
SVEKNQGTQQRQLLSRLQIDPVMAETLQMSQDYYDMESMVHADTRSFILKKPKLSEEIVV
APNQESGMKTADSLRVLSGHLMQTRDLVQPDKPASPKFIVTLDGVPSPPGYMSDQEEDMC
FEGMKPVNQTAASNKGLRGLLHPQQLHLLSRQLEDPNGSFSNAEMSELSVAQKPEKLLER
CKYWPACKNGDECAYHHPISPCKAFPNCKFAEKCLFVHPNCKYDAKCTKPDCPFTHVSRR
IPVLSPKPAVAPPAPPSSSQLCRYFPACKKMECPFYHPKHCRENTQCTRPDCTFYHPTIN
VPPRHALKWIRPQTSE;
(SEQ ID NO: 10, Equus caballus)
MEIGTEISRKIRSAIKGKLQELGAYVDEELPDYIMVMVANKKSQDQMTEDLSLFLGNNTI
RFTVWLHGVLDKLRSVTTDPSSLKSSDTNIFDNNVPSNKSNESRGDERRHEAAVPPLAVS
STRPEKRDSRVSTSSQEQKTTSVRQTYDDGAATRLMSTVKPLREPAPSEDVIDIKPEPDD
LIDEDLNFVQENPLSQKKPTVTLTYGSSRPSIEIYRPPASRNADSGAHLNRLQFQQQQNS
IRAVKQLDMQNSRVYETGRLCEPEVLNSLEETYSPFERNNAEKMSIEEENFRKRKLPVVS
SVVKVKKFNHDGEEEEEDDDCGSRTGSISSSVSVPAKPERRPSLPPSKQANKNLILKAIS
EAQESVTKTTNYSTVSQKQTLPVAPRTRTSQEELLAEMVQGQSKTPRISPPVKEEEIKGD
NIEKSQGTQQRQLLSRLQIDPVVAETLQISQDYYDMESMVHADTRSFILKKPKLSEEIVV
APNQESGMKTADTLRVLSGHLMQTRDLVQPDKPASPKFIVTLDGVPSPPGYMSDQEEDMC
FEGMKPVNQTAASNKGLRGLLHPQQLHLMSRQLDDPNGSFSNAEMSELSVAQKPEKLLER
CKYWPACKNGDECAYHHPVSPCKAFPNCKFAEKCLFVHPNCKYDAKCTKPDCPFTHMSRR
IPVLPPKPAVTTPASPSSSQLCRYFPACKKMECPFYHPKHCRENTQCTRPDCTFYHPTIT
VPPRHALKWIRPQTSE;
(SEQ ID NO: 11, Sus scrofa)
MEIGTEISRKIRSAIKGKLQELGAYVDEELPDYIMVMVANKKSQDQMTEDLSLFLGNNTI
RFTVWLHGVLDKLRSVTTDPSSFKSSDTNIFDSNVPSNKSSFSRGDERRHEAAVPPLAVS
STRPEKRDSRVSTSSQEQKATNVRQTYDDGAATRLMSTVKPLREPAPSEDVIDIKPEPDD
LIDEDLNFVQENPLSQKKPTVTLTYGSSRPSIEIYRPPASRNADSGAHLNRLQFQQQQNS
IHAAKQVDIQSSRVYETGRLCEPEVLNSLEETYSPFFRNNSEKMSIEEENFRKRKLPVVS
SVVKVKKFNHDGEEEEEEDDCGSRTGSISSSVSVPAKPERRPSLPPSKQANKNLILKAIS
EAQESVTKTTNYSTVSQKQTLPVAPRTRTSQEELLAEMVQGQSKAPRISSPIKEEETKGD
NIDKSQGTQQRQLLSRLQIDPVMAETLQISQDYYDMESMVHADTRSFILKKPKLSEEIVV
APNQESGMKTADTLRVLSGHLMQTRDLVQPDKPASPKFIVTLDGVPSPPGYMSDQEEDMC
FEGMRPAHHTAASHEGLAGLLHPQQLHLLSRQLEDPDGSESNAEMSELSVAQKPEKLLER
CKYWPACKNGDECAYHHPISPCKAFPNCKFAEKCLFVHPNCKYDAKCTKPDCPFTHMSRR
IPVLPPKPVTTPAPPSSSQLCRYFPACKKMECPFYHPKHCRENTQCTRPDCTFYHPTITV
PPRHALKWIRPQTSE;
(SEQ ID NO: 12, Oryctolagus cuniculus)
MEIGTEISRKIRSAIKGKLQELGAYVDEELPDYIMVMVANKKSQDQMTEDLSLFLGNNTI
RFTVWLHGVLDKLRSVTTEPSSLKSSDTNIFDSNVPSTKSSFSRGDERRHEATVPPLAVS
SSRPEKRDSRVSTSSQEQKTTVRQTYDDGAATRLMSTVKPLREPAPSEDVIDIKPEPDDL
IDEDLNFVQENPLSQKKPTVTLTYGSSRPSIEIYRPPASRSSDGGAHLNRLQFQQQQNSG
HAGKQLDAQNSRVYETGRLCEPEVLSSLEETYSPFFKNSSEKMGIEDENFRKRKLPVVSS
VVKVKKFNLDGEEEEEDDDYGSRTGSVSSSVSVPAKPERRPSLPPSKQANKNLILKAISE
AQESVTKTTNYSAVPQKQTLPVAPRTRTSQEELLPEVVQGQSRIPRISPPMKEEETKGDN
IEKSQGTQQRQLLSRLQIDPAMAETLQISQDYYDMESMVHADTRSFILKKPKLSEEIVVA
PNQESGMKTADTLRVLSGHLMQTRDLVQPDKPASPKFIVTLDGVPSPPGYMSDQEEDMCF
EGVKPVNQTAASHQGLRGLLHPQQLQLMSRQLDDPDGSFANAEMSELSVAQKPEKLLERC
KYWPACKNGDECAYHHPVSPCKAFPNCKFAEKCLFVHPNCKYDAKCTKPDCPFTHVNRRI
PALPPKPAVTTPGIPSSSQLCRYFPACKKMECPFYHPKHCRENTQCTRPDCTFYHPTITV
PPRHALKWIRPQTSE;
(SEQ ID NO: 13, Cavia porcellus)
MEIGTEISRKIRSAIKGKLQELGAYVDEELPDYIMVMVANKKSQDQMTEDLSLFLGNNTI
RFTVWLHGVLDKLRSVTTEPSSLKSSDTNIFDNNVPSNKSSFSRGDERRHEATVPPLAIS
STRPEKRDSRVAASSQEQKTSNLRQTYDDGAATRLMSTVKPLREPAPSEDVIDIKPEPDD
LIDEDLNFVPENPLSQKKPTVTLTYGSSRPSIEIYRPPASRNAESSAHLNRLQFQQQQNS
IHAAKQLDMQNSRVYETEHTCEPEVLNSLEETYSPFFRNNSEKMMIEEENFRKRKLPVVS
SVVKVKKFNHDGEEEEEDDDYGSRTGSVSSSVSVPAKPERRPSLPPSKQANKNLILKAIS
EAQESVTKTTNYSAVPQKQTLPVAPRTRTSQEELLAEVVQGQSRTSRVSPPIKEEETKGD
NAEKNQGAQQRQLLSRLQIDPVMAETLQSQDYYDMESMVHADTRSFILKKPKLSEEIVVA
PNQESGMKTADTLRVLSGHLMQTRDLVQPDKPASPKFIVTLDGVPSPPGYMSDQEEDMCF
EGVKPVNQTAASNKGLRGLLHPQQLQLMQRQLGDPDGSFSYVEMSELNVAQKPEKLLERC
KYWPACKNGDECAYHHPVSPCKAFPNCKFAEKCLFVHPNCKYDAKCTKPDCPFTHINRRI
PVLPPKPAVTTTASPSTSQLCRYFPACKKMECPFYHPKHCRENTQCTRPDCTFYHPTITV
PPRHALKWIRPQTSE;
(SEQ ID NO: 14, Cricetulus griseus)
MEIGTEISRKIRSAIKGKLQELGAYVDEELPDYIMVMVANKKSQDQMTEDLSLFLGNNTI
RFTVWLHGVLDKLRSVTTEPSSLKSSDTSIFDSNVPSNKSSFSRGDERRHEAAVPPLAVA
SSRPEKRESRVSATSQEQKSTHVRHSYDDGGSTRLMSTVKPLREPAPSEDVIDIKPEPDD
IDEDLNFVQENPLSQKKPTVTLTYGSSRPSIEIYRPPASRNADTGTHLNRLQLHQQQNST
HTAKQLDVQSSQVYEAGRLCEPEVLSSVDDTYSPFFRNNLDKMSIEDENFRKRKLPVVSS
VVKVKRFSHDGEEEEEDEDYGSRIGSLSSSVSVPAKPERRPSLPPSKQANKNLILKAISE
AQESVTKTTNYSAVPQKQTLPVAPRTRTSQEELIAEMVQGQSRAPRISPPVKEEEAKGDS
TEKIQGTQQRQLLSRLQIDPVMVETMEMSPDYYDMESMVHADTRSFILKKPKLSEEIVVT
PNQESGMKTADALRVLSGHLMQTRDLVQPDKPASPKFIVTLDGVPSPPGYMSDQEEEMCF
EGVKPVNQTAASNKGLRGLLHPQHLHLMSRQLEDPDGSFSNAEMTDLSVAQKPEKLLERC
KYWPACKNGDECAYHHPISPCKAFPNCKFAEKCLFVHPNCKYDAKCTKPDCPFTHMSRRI
PVLASKPVVSSPAPSSNGQLCRYFPACKKMECPFYHPKHCRENTQCTRPDCTFYHPTITV
PPRHALKWIRPQTSE;
and
(SEQ ID NO: 15, Felis catus)
MEIGTEISRKIRSAIKGKLQELGAYVDEELPDYIMVMVANKKSQDQMTEDLSLFLGNNTI
RFTVWLHGVLDKLRSVTTDPSSLKSSDTNIFDNNVSSNKSSFGRGDERRHEAAVPPLAVA
STRPEKRDSRVSTSSQEQKTTNVRQTYDDGAATRLMSTVKPLREPAPSEDVIDIKPEPDD
LIDEDLNFVQENPLSQKKPTVTLTYGSSRPSIEIYRPPASRNADSGAHLNRLQFQQQQNN
IHAAKQPDLQNSRVYETGRLCEQEVLNSLEETYSPFFRNNSEKMSIEEENFRKRKLPVVS
SVVKVKKFNHDGEEEEEDDDCGSRTGSISSSVSVPAKPERRPSLPPSKQANKNLILKAIS
EAQESVTKTTNYSTVSQKQTLPVAPRTRTSQEELLAEMVQGQSRTPRISPPIKEEETKGD
NIEKSQGAPQRQLLSRLQIDPVMAETLQISQDYYDMESMVHADTRSFILKKPKLSEEIVV
APNQESGMKTADTLRVLSGHLMQTRDLVQPDKPASPKFIVTLDGVPSPPGYMSDQEEDMC
FEGMKPVNQTAAASKGLRGLLHPQQLQLMSRQLDDPNGSFANAEMSELSVAQKPEKLLER
CKYWPACKNGDECAYHHPVSPCKAFPNCKFAEKCLFVHPNCKYDAKCTKPDCPFTHMSRR
IPVLPLKPAVTTPAPPSSSQLCRYFPACKKMECPFYHPKHCRENTQCTRPDCTFYHPTIT
VPPRHALKWIRPQTSE.

In some embodiments, the first domain comprises an amino acid sequence having at least 90%, 91%, 92%, 93%, 94%, 95%, 96%, 97%, 98% or 99% identity to an amino acid sequence selected from the group consisting of SEQ ID NOs: 1-15. For example, the first domain comprises an amino acid sequence having at least 95%, 96%, 97%, 98% or 99% identity to an amino acid sequence selected from the group consisting of SEQ ID NOs: 1-15. In some preferred embodiments, the first domain comprises an amino acid sequence having at least 97%, 98% or 99% identity to an amino acid sequence selected from the group consisting of SEQ ID NOs: 1-15.

In some preferred embodiments, the first domain comprises an amino acid sequence having at least 95%, 96%, 97%, 98% or 99% identity to SEQ ID NO: 1.

In some embodiments of any one of the aspects, the amino acid sequence of the first domain comprises a mutation at position 475 of the human ZC3H14 or a homologous or orthologous ZC3H14 protein. For example, the first domain comprises an amino acid sequence having a mutation at position 475 of the human ZC3H14 amino acid sequence, e.g., SEQ ID NO: 1, or a corresponding position in a homologous or orthologous ZC3H14 protein.

In some embodiments, the first domain comprises a phosphoserine mimetic at position 475 of the human ZC3H14 amino acid sequence, e.g., SEQ ID NO: 1, or a corresponding position in a homologous or orthologous ZC3H14 protein. For example, the first domain comprises aspartic acid, glutamic acid, phosphothreonine or a non-hydrolyzable phosphoserine mimetic at position 475 of the human ZC3H14 amino acid sequence, e.g., SEQ ID NO: 1, or a corresponding position in a homologous or orthologous ZC3H14 protein. In some preferred embodiments, the first domain comprises aspartic acid at position 475 of the human ZC3H14 amino acid sequence, e.g., SEQ ID NO: 1, or a corresponding position in a homologous or orthologous ZC3H14 protein.

In some embodiments, the first domain comprises a non-phosphoserine mimetic at position 475 of the human ZC3H14 amino acid sequence, e.g., SEQ ID NO: 1, or a corresponding position in a homologous or orthologous ZC3H14 protein. For example, the first domain comprises alanine, cysteine, selenocystine, threonine, methoine, glycine, valine, leucine or isoleucine at position 475 of the human ZC3H14 amino acid sequence, e.g., SEQ ID NO: 1, or a corresponding position in a homologous or orthologous ZC3H14 protein. In some preferred embodiments, the first domain comprises alanine at position 475 of the human ZC3H14 amino acid sequence, e.g., SEQ ID NO: 1, or a corresponding position in a homologous or orthologous ZC3H14 protein.

In some preferred embodiments, the first domain comprises a S->D, S->A or S->E mutation at position 475 of the human ZC3H14 amino acid sequence, e.g., SEQ ID NO: 1, or a corresponding position in a homologous or orthologous ZC3H14 protein.

In some embodiments, the first domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the group consisting of:

(SEQ ID NO: 16, ZC3H14 S475D)
MEIGTEISRKIRSAIKGKLQELGAYVDEELPDYIMVMVANKKSQDQMTEDLSLFL
GNNTIRFTVWLHGVLDKLRSVTTEPSSLKSSDTNIFDSNVPSNKSNFSRGDERRHE
AAVPPLAIPSARPEKRDSRVSTSSQESKTTNVRQTYDDGAATRLMSTVKPLREPA
PSEDVIDIKPEPDDLIDEDLNFVQENPLSQKKPTVTLTYGSSRPSIEIYRPPASRNAD
SGVHLNRLQFQQQQNSIHAAKQLDMQSSWVYETGRLCEPEVLNSLEETYSPFFR
NNSEKMSMEDENFRKRKLPVVSSVVKVKKFNHDGEEEEEDDDYGSRTGSISSSV
SVPAKPERRPSLPPSKQANKNLILKAISEAQESVTKTTNYSTVPQKQTLPVAPRTR
TSQEELLAEVVQGQSRTPRISPPIKEEETKGDSVEKNQGTQQRQLLSRLQIDPVMA
ETLQMSQDYYDMESMVHADTRSFILKKPKLDEEVVVAPNQESGMKTADSLRVL
SGHLMQTRDLVQPDKPASPKFIVTLDGVPSPPGYMSDQEEDMCFEGMKPVNQTA
ASNKGLRGLLHPQQLHLLSRQLEDPNGSFSNAEMSELSVAQKPEKLLERCKYWP
ACKNGDECAYHHPISPCKAFPNCKFAEKCLFVHPNCKYDAKCTKPDCPFTHVSR
RIPVLSPKPAVAPPAPPSSSQLCRYFPACKKMECPFYHPKHCRFNTQCTRPDCTFY
HPTINVPPRHALKWIRPQTSE;
or
(SEQ ID NO: 17, ZC3H14 S475E)
MEIGTEISRKIRSAIKGKLQELGAYVDEELPDYIMVMVANKKSQDQMTEDLSLFL
GNNTIRFTVWLHGVLDKLRSVTTEPSSLKSSDTNIFDSNVPSNKSNFSRGDERRHE
AAVPPLAIPSARPEKRDSRVSTSSQESKTTNVRQTYDDGAATRLMSTVKPLREPA
PSEDVIDIKPEPDDLIDEDLNFVQENPLSQKKPTVTLTYGSSRPSIEIYRPPASRNAD
SGVHLNRLQFQQQQNSIHAAKQLDMQSSWVYETGRLCEPEVLNSLEETYSPFFR
NNSEKMSMEDENFRKRKLPVVSSVVKVKKFNHDGEEEEEDDDYGSRTGSISSSV
SVPAKPERRPSLPPSKQANKNLILKAISEAQESVTKTTNYSTVPQKQTLPVAPRTR
TSQEELLAEVVQGQSRTPRISPPIKEEETKGDSVEKNQGTQQRQLLSRLQIDPVMA
ETLQMSQDYYDMESMVHADTRSFILKKPKLEEEVVVAPNQESGMKTADSLRVL
SGHLMQTRDLVQPDKPASPKFIVTLDGVPSPPGYMSDQEEDMCFEGMKPVNQTA
ASNKGLRGLLHPQQLHLLSRQLEDPNGSFSNAEMSELSVAQKPEKLLERCKYWP
ACKNGDECAYHHPISPCKAFPNCKFAEKCLFVHPNCKYDAKCTKPDCPFTHVSR
RIPVLSPKPAVAPPAPPSSSQLCRYFPACKKMECPFYHPKHCRFNTQCTRPDCTFY
HPTINVPPRHALKWIRPQTSE;
(SEQ ID NO: 18, ZC3H14 S475A)
MEIGTEISRKIRSAIKGKLQELGAYVDEELPDYIMVMVANKKSQDQMTEDLSLFL
GNNTIRFTVWLHGVLDKLRSVTTEPSSLKSSDTNIFDSNVPSNKSNFSRGDERRHE
AAVPPLAIPSARPEKRDSRVSTSSQESKTTNVRQTYDDGAATRLMSTVKPLREPA
PSEDVIDIKPEPDDLIDEDLNFVQENPLSQKKPTVTLTYGSSRPSIEIYRPPASRNAD
SGVHLNRLQFQQQQNSIHAAKQLDMQSSWVYETGRLCEPEVLNSLEETYSPFFR
NNSEKMSMEDENFRKRKLPVVSSVVKVKKFNHDGEEEEEDDDYGSRTGSISSSV
SVPAKPERRPSLPPSKQANKNLILKAISEAQESVTKTTNYSTVPQKQTLPVAPRTR
TSQEELLAEVVQGQSRTPRISPPIKEEETKGDSVEKNQGTQQRQLLSRLQIDPVMA
ETLQMSQDYYDMESMVHADTRSFILKKPKLAEEVVVAPNQESGMKTADSLRVL
SGHLMQTRDLVQPDKPASPKFIVTLDGVPSPPGYMSDQEEDMCFEGMKPVNQTA
ASNKGLRGLLHPQQLHLLSRQLEDPNGSFSNAEMSELSVAQKPEKLLERCKYWP
ACKNGDECAYHHPISPCKAFPNCKFAEKCLFVHPNCKYDAKCTKPDCPFTHVSR
RIPVLSPKPAVAPPAPPSSSQLCRYFPACKKMECPFYHPKHCRFNTQCTRPDCTFY
HPTINVPPRHALKWIRPQTSE.

In some embodiments, the first domain comprises an amino acid sequence having at least 90%, 91%, 92%, 93%, 94%, 95%, 96%, 97%, 98% or 99% identity to an amino acid sequence selected from the group consisting of SEQ ID NOs: 16-18. For example, the first domain comprises an amino acid sequence having at least 95%, 96%, 97%, 98% or 99% identity to an amino acid sequence selected from the group consisting of SEQ ID NOs: 16-18. In some preferred embodiments, the first domain comprises an amino acid sequence having at least 97%, 98% or 99% identity to an amino acid sequence selected from the group consisting of SEQ ID NOs: 16-18. For example, the first domain comprises an amino acid sequence having 100% identity to an amino acid sequence selected from the group consisting of SEQ ID NOs: 16-18.

### RNA Binding Moiety

In some embodiments of any one of the aspects, the polypeptide can be linked to a nucleic acid binding moiety, e.g., a moiety for binding to an RNA molecule.

In some embodiments of any one of the aspects, the nucleic acid binding moiety lacks nuclease activity.

In some embodiments, the nucleic acid binding moiety comprises at least a nucleic acid binding domain of a nucleic acid binding protein. For example, the polypeptide comprises a first domain comprising an amino acid sequence having at least 80% identity to the amino acid sequence of a wild-type ZC3H14 and a second domain comprising an amino acid sequence having at least 80% identity to the amino acid sequence of a nucleic acid binding domain of a nucleic acid binding protein. Some exemplary nucleic acid binding proteins include, but are not limited to, clustered regularly interspaced short palindromic repeats (CRISPR)-associated (Cas) proteins, zinc finger nucleases (ZFNs), transcription activator-like effector-based nucleases (TALENs), Argonaute proteins, p53, Jun, Fos, GCN4, GAL4, RAP1, and LexA.

It is noted that nucleic acid binding domain can be from a DNA binding protein or an RNA binding protein. Further, the nucleic acid binding domain can be non-specific or sequence specific binding domain. In some preferred embodiments, the nucleic acid binding domain is from an RNA binding protein.

### CasRX

In some embodiments of any one of the aspects, the nucleic acid binding domain is from a CRISPR/Cas protein.

Exemplary CRISPR/Cas protein include, but are not limited to, CasRX, Casl, CaslB, Cas2, Cas3, Cas4, Cas5, Cas6, Cas7, Cas8, Cas9 (also known as Csnl and Csxl2), CaslO, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, CsxlO, Csx16, CsaX, Csx3, Csx1, Csx15, Csf1, Csf2, Csf3, Csf4, and homologues or orthologues thereof, or modified versions thereof.

In some embodiments of any one of the aspects, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to CasRX, or a homologous or orthologous of CasRX. For example, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity CasRX or a homologous or orthologous of CasRX.

In some embodiments, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence encoded by a nucleic acid comprising the sequence (SEQ ID NO: 19):

atcgaaaaaaaaaagtccttcgccaagggcatgggcgtgaagtccacactcgtgtccggctccaaagtgtacatgacaaccttcgccgaag
gcagcgacgccaggctggaaaagatcgtggagggcgacagcatcaggagcgtgaatgagggcgaggccttcagcgctgaaatggccg
ataaaaacgccggctataagatcggcaacgccaaattcagccatcctaagggctacgccgtggtggctaacaaccctctgtatacaggacc
cgtccagcaggatatgctcggcctgaaggaaactctggaaaagaggtacttcggcgagagcgctgatggcaatgacaatatttgtatccag
gtgatccataacatcctggacattgaaaaaatcctcgccgaatacattaccaacgccgcctacgccgtcaacaatatctccggcctggataag
gacattattggattcggcaagttctccacagtgtatacctacgacgaattcaaagaccccgagcaccatagggccgctttcaacaataacgat
aagctcatcaacgccatcaaggcccagtatgacgagttcgacaacttcctcgataaccccagactcggctatttcggccaggcctttttcagc
aaggagggcagaaattacatcatcaattacggcaacgaatgctatgacattctggccctcctgagcggactgaggcactgggtggtccataa
caacgaagaagagtccaggatctccaggacctggctctacaacctcgataagaacctcgacaacgaatacatctccaccctcaactacctct
acgacaggatcaccaatgagctgaccaactccttctccaagaactccgccgccaacgtgaactatattgccgaaactctgggaatcaaccct
gccgaattcgccgaacaatatttcagattcagcattatgaaagagcagaaaaacctcggattcaatatcaccaagctcagggaagtgatgctg
gacaggaaggatatgtccgagatcaggaaaaatcataaggtgttcgactccatcaggaccaaggtctacaccatgatggactttgtgatttata
ggtattacatcgaagaggatgccaaggtggctgccgccaataagtccctccccgataatgagaagtccctgagcgagaaggatatctttgtg
attaacctgaggggctccttcaacgacgaccagaaggatgccctctactacgatgaagctaatagaatttggagaaagctcgaaaatatcatg
cacaacatcaaggaatttaggggaaacaagacaagagagtataagaagaaggacgcccctagactgcccagaatcctgcccgctggccg
tgatgtttccgccttcagcaaactcatgtatgccctgaccatgttcctggatggcaaggagatcaacgacctcctgaccaccctgattaataaat
tcgataacatccagagcttcctgaaggtgatgcctctcatcggagtcaacgctaagttcgtggaggaatacgcctttttcaaagactccgccaa
gatcgccgatgagctgaggctgatcaagtccttcgctagaatgggagaacctattgccgatgccaggagggccatgtatatcgacgccatcc
gtattttaggaaccaacctgtcctatgatgagctcaaggccctcgccgacaccttttccctggacgagaacggaaacaagctcaagaaaggc
aagcacggcatgagaaatttcattattaataacgtgatcagcaataaaaggttccactacctgatcagatacggtgatcctgcccacctccatg
agatcgccaaaaacgaggccgtggtgaagttcgtgctcggcaggatcgctgacatccagaaaaaacagggccagaacggcaagaacca
gatcgacaggtactacgaaacttgtatcggaaaggataagggcaagagcgtgagcgaaaaggtggacgctctcacaaagatcatcaccgg
aatgaactacgaccaattcgacaagaaaaggagcgtcattgaggacaccggcagggaaaacgccgagagggagaagtttaaaaagatca
tcagcctgtacctcaccgtgatctaccacatcctcaagaatattgtcaatatcaacgccaggtacgtcatcggattccattgcgtcgagcgtgat
gctcaactgtacaaggagaaaggctacgacatcaatctcaagaaactggaagagaagggattcagctccgtcaccaagctctgcgctggca
ttgatgaaactgcccccgataagagaaaggacgtggaaaaggagatggctgaaagagccaaggagagcattgacagcctcgagagcgc
caaccccaagctgtatgccaattacatcaaatacagcgacgagaagaaagccgaggagttcaccaggcagattaacagggagaaggcca
aaaccgccctgaacgcctacctgaggaacaccaagtggaatgtgatcatcagggaggacctcctgagaattgacaacaagacatgtaccct
gttcagaaacaaggccgtccacctggaagtggccaggtatgtccacgcctatatcaacgacattgccgaggtcaattcctacttccaactgta
ccattacatcatgcagagaattatcatgaatgagaggtacgagaaaagcagcggaaaggtgtccgagtacttcgacgctgtgaatgacgag
aagaagtacaacgataggctcctgaaactgctgtgtgtgcctttcggctactgtatccccaggtttaagaacctgagcatcgaggccctgttcg
ataggaacgaggccgccaagttcgacaaggagaaaaagaaggtgtccggcaattcc

In some embodiments, the second domain comprises an amino acid sequence having at least 90%, 91%, 92%, 93%, 94%, 95%, 96%, 97%, 98% or 99% identity to an amino acid sequence encoded by a nucleic acid comprising SEQ ID NO: 19. In some embodiments, the second domain comprises an amino acid sequence having at least 95%, 96%, 97%, 98% or 99% identity to an amino acid sequence encoded by a nucleic acid comprising SEQ ID NO: 19. In some embodiments, the second domain comprises an amino acid sequence having at least 97%, 98% or 99% identity to an amino acid sequence encoded by a nucleic acid comprising SEQ ID NO: 19. In some embodiments, the second domain comprises an amino acid sequence having 100% identity to an amino acid sequence encoded by a nucleic acid comprising SEQ ID NO: 19.

### Ago Domain

In some embodiments of any one of the aspects described herein, the second domain comprises at least the nucleic acid binding domain of an Argonaute protein. Argonaute proteins are proteins of the PIWI protein superfamily that contain an N-terminal (N), a Piwi-Argonaute-Zwille (PAZ), a middle (MID), and a P-element-induced wimpy testis (PIWI) domain. Ago are capable of binding small RNAs, such as microRNAs, small interfering RNAs (siRNAs), and Piwi-interacting RNAs. Agos can be guided to target sequences with these RNAs in order to cleave mRNA, inhibit translation, or induce mRNA degradation in the target sequence. Generally, the domains are connected in some arrangements by structured linker regions. Agos possessing this structural layout, which include prokaryotic and eukaryotic Agos, are considered “long.” However, there also exists a class of “short” Agos which only possess MID and PIWI domain. The 5′ end of the guide is sequestered in a region of the MID domain. While the residues involved in this binding are somewhat conserved, some marked differences exist between eukaryotic Agos and prokaryotic Agos. The 3′ end of the guide is bound by the PAZ domain. The catalytic region of Agos is an RNase H-like fold located in the PIWI domain, which utilized a conserved DEDX (X=D or H) tetrad for catalysis. Mutations to these residues renders the Argonaute inactive. Also included in the Argonaute family of proteins as described herein are the Piwi subfamily of proteins such as Hili, Hiwi, Hiwi 2 and Hiwi3.

The mammalian Ago family comprises eight members, four of which are ubiquitously expressed (Ago subfamily), with the remaining four (Piwi subfamily) being expressed in germ cells. While Ago2 has been shown to be at the core of the RISC complex that carries out oligonucleotide-guided target RNA cleavage in the region of complementarity, Ago1, 3, and 4 are thought to lack this cleavage activity and may therefore function in related oligonucleotide-guided gene silencing pathways that do not involve target RNA cleavage in the region of complementarity. Similarly, Ago2 may function in gene silencing independent of such cleavage activity, such as in translational repression.

In some embodiments of any one of the aspects, Ago protein can be from Anoxybacillus flavithermus, Aquifex aeolicus, Aquifex aeolicus strain VF5, Arabidopsis thaliana, Archaeoglobus fulgidus, Aromatoleum aromaticum, Clostridium bartlettii, D. melanogaster, Exiguobacterium, Halogeometricum borinquense, Halorubrum lacusprofundi, Microsystis aeruginosa, Pyrococcus furiosus, Synechococcus, Synechococcus elongatus, Thermosynechococcus elogatus, Thermus thermophilus, Thermus thermophilus JL-18, or Thermus thermophilus strain HB27.

Exemplary sequences for Agos can be found in Genebank with Accession Numbers as listed: human Ago1 (NP 036331); human Ago2 (NP 036286), human Ago3 (NP 079128), human Ago4 (NP 060099) Hili (NP 060538), Hiwi (NP 0047553), Hiwi2 (NP 689644), Hiwi3 (NP 001008496), Drosophila melanogaster (Dm) Ago 1 (NP 725341), Dm Ago2 (NP 730054), Dm Ago3 (ABO27430), Aubergine (CAA64320), PIWI (NP 476875), Arahidopsis thalicma (At) Ago1 (NP 849784), At Ago2 (NP 174413), At Ago3 (NP 174414), At Ago4 (NP 565633), At Ago5 (At2g27880), At Ago6 (At2g32940), At Ago7 (NP 1771033), At Ago8 (NP 1976023), At Ago9 (CAD66636), At Ago 10 (NP 199194), Shizosaccharomyces pombe (Sp) Ago (NP 587782) and Caenorhabidilis elegans (Ce) Alg-1 (fNP 5103221).

In some embodiments of any one of the aspects, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a mammalian Ago. For example, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to a hAgo1, hAgo2, hAgo3, hAgo4 or a homologous or orthologous Ago protein.

In some embodiments, the second domain comprises an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% identity to an amino acid sequence selected from the group consisting of:

(SEQ ID NO: 20, hAgo1)
MEAGPSGAAAGAYLPPLQQVFQAPRRPGIGTVGKPIKLLANYFEVDIPKIDVYHYEVDIK
PDKCPRRVNREVVEYMVQHFKPQIFGDRKPVYDGKKNIYTVTALPIGNERVDFEVTIPGE
GKDRIFKVSIKWLAIVSWRMLHEALVSGQIPVPLESVQALDVAMRHLASMRYTPVGRSFF
SPPEGYYHPLGGGREVWFGFHQSVRPAMWKMMLNIDVSATAFYKAQPVIEFMCEVLDIRN
IDEQPKPLTDSQRVRFTKEIKGLKVEVTHCGQMKRKYRVCNVTRRPASHQTFPLQLESGQ
TVECTVAQYFKQKYNLQLKYPHLPCLQVGQEQKHTYLPLEVCNIVAGQRCIKKLTDNQTS
TMIKATARSAPDRQEEISRLMKNASYNLDPYIQEFGIKVKDDMTEVTGRVLPAPILQYGG
RNRAIATPNQGVWDMRGKQFYNGIEIKVWAIACFAPQKQCREEVLKNFTDQLRKISKDAG
MPIQGQPCFCKYAQGADSVEPMFRHLKNTYSGLQLIIVILPGKTPVYAEVKRVGDTLLGM
ATQCVQVKNVVKTSPQTLSNLCLKINVKLGGINNILVPHQRSAVFQQPVIFLGADVTHPP
AGDGKKPSITAVVGSMDAHPSRYCATVRVQRPRQEIIEDLSYMVRELLIQFYKSTRFKPT
RIIFYRDGVPEGQLPQILHYELLAIRDACIKLEKDYQPGITYIVVQKRHHTRLFCADKNE
RIGKSGNIPAGTTVDTNITHPFEFDFYLCSHAGIQGTSRPSHYYVLWDDNRFTADELQIL
TYQLCHTYVRCTRSVSIPAPAYYARLVAFRARYHLVDKEHDSGEGSHISGQSNGRDPQAL
AKAVQVHQDTLRTMYFA,
(SEQ ID NO: 21, hAgo2)
MYSGAGPALAPPAPPPPIQGYAFKPPPRPDFGTSGRTIKLQANFFEMDIPKIDIYHYELD
IKPEKCPRRVNREIVEHMVQHFKTQIFGDRKPVFDGRKNLYTAMPLPIGRDKVELEVTLP
GEGKDRIFKVSIKWVSCVSLQALHDALSGRLPSVPFETIQALDVVMRHLPSMRYTPVGRS
FFTASEGCSNPLGGGREVWFGFHQSVRPSLWKMMLNIDVSATAFYKAQPVIEFVCEVLDE
KSIEEQQKPLTDSQRVKFTKEIKGLKVEITHCGQMKRKYRVCNVTRRPASHQTFPLQQES
GQTVECTVAQYFKDRHKLVLRYPHLPCLQVGQEQKHTYLPLEVCNIVAGQRCIKKLTDNQ
TSTMIRATARSAPDRQEEISKLMRSASENTDPYVREFGIMVKDEMTDVTGRVLQPPSILY
GGRNKAIATPVQGVWDMRNKQFHTGIEIKVWAIACFAPQRQCTEVHLKSFTEQLRKISRD
AGMPIQGQPCFCKYAQGADSVEPMFRHLKNTYAGLQLVVVILPGKTPVYAEVKRVGDTVL
GMATQCVQMKNVQRTTPQTLSNLCLKINVKLGGVNNILLPQGRPPVFQQPVIFLGADVTH
PPAGDGKKPSIAAVVGSMDAHPNRYCATVRVQQHRQEIIQDLAAMVRELLIQFYKSTREK
PTRIIFYRDGVSEGQFQQVLHHELLAIREACIKLEKDYQPGITFIVVQKRHHTRLFCTDK
NERVGKSGNIPAGTTVDTKITHPTEFDFYLCSHAGIQGTSRPSHYHVLWDDNRESSDELQ
ILTYQLCHTYVRCTRSVSIPAPAYYAHLVAFRARYHLVDKEHDSAEGSHTSGQSNGRDHQ
ALAKAVQVHQDTLRTMYFA,
(SEQ ID NO: 22, hAgo3)
MEIGSAGPAGAQPLLMVPRRPGYGTMGKPIKLLANCFQVEIPKIDVYLYEVDIKPDKCPR
RVNREVVDSMVQHFKVTIFGDRRPVYDGKRSLYTANPLPVATTGVDLDVTLPGEGGKDRP
FKVSIKFVSRVSWHLLHEVLTGRTLPEPLELDKPISTNPVHAVDVVLRHLPSMKYTPVGR
SFFSAPEGYDHPLGGGREVWFGFHQSVRPAMWKMMLNIDVSATAFYKAQPVIQFMCEVLD
IHNIDEQPRPLTDSHRVKFTKEIKGLKVEVTHCGTMRRKYRVCNVTRRPASHQTFPLQLE
NGQTVERTVAQYFREKYTLQLKYPHLPCLQVGQEQKHTYLPLEVCNIVAGQRCIKKLTDN
QTSTMIKATARSAPDRQEEISRLVRSANYETDPFVQEFQFKVRDEMAHVTGRVLPAPMLQ
YGGRNRTVATPSHGVWDMRGKQFHTGVEIKMWAIACFATQRQCREEILKGFTDQLRKISK
DAGMPIQGQPCFCKYAQGADSVEPMFRHLKNTYSGLQLIIVILPGKTPVYAEVKRVGDTL
LGMATQCVQVKNVIKTSPQTLSNLCLKINVKLGGINNILVPHQRPSVFQQPVIFLGADVT
HPPAGDGKKPSIAAVVGSMDAHPSRYCATVRVQRPRQEIIQDLASMVRELLIQFYKSTRF
KPTRIIFYRDGVSEGQFRQVLYYELLAIREACISLEKDYQPGITYIVVQKRHHTRLFCAD
RTERVGRSGNIPAGTTVDTDITHPYEFDFYLCSHAGIQGTSRPSHYHVLWDDNCFTADEL
QLLTYQLCHTYVRCTRSVSIPAPAYYAHLVAFRARYHLVDKEHDSAEGSHVSGQSNGRDP
QALAKAVQIHQDTLRTMYFA,
or
(SEQ ID NO: 23, hAgo4)
MEALGPGPPASLFQPPRRPGLGTVGKPIRLLANHFQVQIPKIDVYHYDVDIKPEKRPRRV
NREVVDTMVRHFKMQIFGDRQPGYDGKRNMYTAHPLPIGRDRVDMEVTLPGEGKDQTFKV
SVQWVSVVSLQLLLEALAGHLNEVPDDSVQALDVITRHLPSMRYTPVGRSFFSPPEGYYH
PLGGGREVWFGFHQSVRPAMWNMMLNIDVSATAFYRAQPIIEFMCEVLDIQNINEQTKPL
TDSQRVKFTKEIRGLKVEVTHCGQMKRKYRVCNVTRRPASHQTFPLQLENGQAMECTVAQ
YFKQKYSLQLKYPHLPCLQVGQEQKHTYLPLEVCNIVAGQRCIKKLTDNQTSTMIKATAR
SAPDRQEEISRLVKSNSMVGGPDPYLKEFGIVVHNEMTELTGRVLPAPMLQYGGRNKTVA
TPNQGVWDMRGKQFYAGIEIKVWAVACFAPQKQCREDLLKSFTDQLRKISKDAGMPIQGQ
PCFCKYAQGADSVEPMFKHLKMTYVGLQLIVVILPGKTPVYAEVKRVGDTLLGMATQCVQ
VKNVVKTSPQTLSNLCLKINAKLGGINNVLVPHQRPSVFQQPVIFLGADVTHPPAGDGKK
PSIAAVVGSMDGHPSRYCATVRVQTSRQEISQELLYSQEVIQDLINMVRELLIQFYKSTR
FKPTRIIYYRGGVSEGQMKQVAWPELIAIRKACISLEEDYRPGITYIVVQKRHHTRLFCA
DKTERVGKSGNVPAGTTVDSTITHPSEFDFYLCSHAGIQGTSRPSHYQVLWDDNCFTADE
LQLLTYQLCHTYVRCTRSVSIPAPAYYARLVAFRARYHLVDKDHDSAEGSHVSGQSNGRD
PQALAKAVQIHHDTQHTMYFA.

In some embodiments, the second domain comprises an amino acid sequence having at least 90%, 91%, 92%, 93%, 94%, 95%, 96%, 97%, 98% or 99% identity to an amino acid sequence selected from the group consisting of SEQ ID NOs: 20-23. In some embodiments, the second domain comprises an amino acid sequence having at least 95%, 96%, 97%, 98% or 99% identity to an amino acid sequence selected from the group consisting of SEQ ID NOs: 20-23. In some embodiments, the second domain comprises an amino acid sequence having at least 97%, 98% or 99% identity to an amino acid sequence selected from the group consisting of SEQ ID NOs: 20-23. In some embodiments, the second domain comprises an amino acid sequence having 100% identity to an amino acid sequence selected from the group consisting of SEQ ID NOs: 20-23.

In some embodiments, the second domain comprises an amino acid sequence having a mutation at one or more positions selected from the group consisting of D597 and D699 of human Ago2 amino acid sequence, e.g., SEQ ID NO: 21, or a corresponding position in a homologous or orthologous Ago protein. For example, the second domain comprises an amino acid sequence having a mutation at one or more positions selected from the group consisting of D597A and D699A of human Ago2 amino acid sequence, e.g., SEQ ID NO: 21, or a corresponding position in a homologous or orthologous Ago protein.

### Linkers

In some embodiments of any of the aspects, the polypeptide comprises a linker between the first domain and the nucleic acid binding moiety, e.g., the second domain. The linker can be a chemical linker, a single peptide bond (e.g., linked directly to each other) or a peptide linker containing one or more amino acid residues (e.g. with an intervening amino acid or amino acid sequence between the first and second domains).

In some embodiments of any of the aspects, the linker used to link the first domain with the nucleic acid binding moiety, e.g., the second domain is a flexible linker. As used herein, a “flexible linker” is a linker which does not have a fixed structure (secondary or tertiary structure) in solution and is therefore free to adopt a variety of conformations. Generally, a flexible linker has a plurality of freely rotating bonds along its backbone. In contrast, a rigid linker is a linker which adopts a relatively well-defined conformation when in solution. Rigid linkers are therefore those which have a particular secondary and/or tertiary structure in solution.

In some embodiments of the various aspects described herein, the first domain and the nucleic acid binding moiety, e.g., the second domain are linked via a peptide linker. The term “peptide linker” as used herein denotes a peptide with amino acid sequences, which is in some embodiments of synthetic origin. It is noted that peptide linkers may affect folding of a given polypeptide, and may also react/bind with other proteins, and these properties can be screened for by known techniques. A peptide linker can comprise 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond. Conversely, a peptide linker can comprise less than 50 amino acids, less than 45 amino acids, less than 40 amino acids, less than 35 amino acids, less than 30 amino acids, less than 30 amino acids, less than 25 amino acids, less than 20 amino acids, less than 15 amino acids or less than 10 amino acids.

In some embodiments of the various aspects described herein, the peptide linker comprises from about 5 amino acids to about 40 amino acids. For example, the peptide linker can comprise from about 5 amino acids to about 35 amino acids, from about 10 amino acids to 30 amino acids, or from about 10 amino acids to about 25 amino acids.

In some embodiments of the various aspects described herein, the linker comprises 10, 11, 12, 13, 14, 15, 16, 17, 18, 19, 20, 21, 22, 23, 24 or 25 amino acids. For example, the linker comprises 10, 11, 12, 13, 14, 15, 16, 17, 18, 19 or 20 amino acids. Preferably, the linker comprises 12, 13, 14, 15, 16, 17 or 18 amino acids. More preferably, the linker comprises 14, 15 or 16 amino acids. In some embodiments of the various aspects described herein, the linker comprises 15 amino acids.

Some exemplary peptide linkers include those that consist of glycine and serine residues, the so-called Gly-Ser polypeptide linkers. As used herein, the term “Gly-Ser polypeptide linker” refers to a peptide that consists of glycine and serine residues. In some embodiments of the various aspects described herein, the peptide linker comprises the amino acid sequence (GlyₓSer)ₙ, where x is 2, 3, 4, 5 or 6, and n is 1, 2, 3, 4, 5, 6, 7, 8, 9 or 10, e.g., SEQ ID NO: 24. In some embodiments of the various aspects described herein, x is 3 and n is 3, 4, 5 or 6. In some embodiments of the various aspects described herein, x is 3 and n is 4 or 5. In some embodiments of the various aspects described herein, x is 4 and n is 3, 4, 5 or 6. In some embodiments of the various aspects described herein, x is 4 and n is 4 or 5. In some embodiments of the various aspects described herein, x is 3 and n is 2. In some embodiments of the various aspects described herein, x is 3 or 4 and n is 1.

More exemplary linkers, in addition to those described herein, include a string of histidine residues, e.g., His6 (HHHHHH (SEQ ID NO: 25)); sequences made up of Ala and Pro, varying the number of Ala-Pro pairs to modulate the flexibility of the linker; and sequences made up of charged amino acid residues e.g., mixing Glu and Lys. Flexibility can be controlled by the types and numbers of residues in the linker. See, e.g., Perham, 30 Biochem. 8501 (1991); Wriggers et al., 80 Biopolymers 736 (2005).

In some embodiments of the various aspects described herein, the linker can be a chemical linker. Chemical linkers can comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, or C(O). The linker can be 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond.

In some embodiments of any one of the aspects, the polypeptide can comprise an epitope or affinity tag, which can provide a convenient means for isolating or purifying the polypeptide. A number of epitope or affinity tags are known in the art. These are usually divided into 3 classes according to their size: small tags have a maximum of 12 amino acids, medium-sized ones have a maximum of 60 and large ones have more than 60. The small tags include the Arg-tag, the His-tag, the avidin biotin, or streptavidin (Strep)-tag, the Flag-tag, the T7-tag, the V5-peptide-tag and the c-Myc-tag, the medium-sized ones include the S-tag, the HAT-tag, the calmodulin-binding peptide, the chitin-binding peptide and some cellulose-binding domains. The latter can contain up to 189 amino acids and are then regarded, like the glutathione-S-transferase (GST)-and maltose binding protein (MBP)-tag, as large affinity tags.

In some embodiments of any one of the aspects, the polypeptide comprises a 6-HIS tag (HHHHHH, SEQ ID NO: 26), a HA tag (YPYDVPDYA, SEQ ID NO: 27), ac-Myc epitope EQKLISEEDL, SEQ ID NO: 28), an AU1 tag (DTYRYI, SEQ ID NO: 29), and/or aFlag-tag (DYKDDDDK, SEQ ID NO: 30).

When present, the epitope or affinity tag can be located anywhere in the polypeptide. For example, the epitope or affinity tag can be at the N-terminal, C-terminal or at an internal position of the polypeptide. In some embodiments, the epitope or affinity tag is at a position N-terminal of the first domain. In some embodiments the epitope or affinity tag is at a position C-terminal of the first domain. In some embodiments, the epitope or affinity tag is at a position N-terminal of the second domain. In some embodiments the epitope or affinity tag is at a position C-terminal of the second domain.

In some embodiments, the epitope or affinity tag is between the first and the second domain. In other words, the epitope or affinity tag is part of the linker linking the first and the second domain. When the epitope or affinity tag is between the first and second domain, there can be a linker between the first domain and the epitope or affinity tag. Similarly, there can also be a linker between second domain and the epitope or affinity tag. In some embodiments, the epitope or affinity tag is at the N-terminal of the polypeptide. In some other embodiments, the epitope or affinity tag is at the C-terminal of the polypeptide.

### Polynucleotide Encoding the Polypeptide

The disclosure also provides a polynucleotide encoding a polypeptide described herein. The skilled person will understand that, due to the degeneracy of the genetic code, a given polypeptide can be encoded by different polynucleotides. These “variants” are encompassed herein.

In some embodiments, a polynucleotide encoding a polypeptide described herein is comprised in a vector. In some embodiments, a nucleic acid sequence encoding a polypeptide described herein is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a polypeptide or a nucleic acid encoding an expression product which is operably linked to at least one non-native (e.g., heterologous) genetic control element (e.g., a promoter, suppressor, activator, enhancer, response element, or the like).

In some embodiments, the vector or polynucleotide described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell. In some embodiments, the vector and/or nucleic acid sequence described herein is codon-optimized for expression in a yeast or yeast cell. In some embodiments, the vector and/or nucleic acid sequence described herein is codon-optimized for expression in a bacterial cell. In some embodiments, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding an antibody or antigen-binding fragment thereof as described herein in place of non-essential viral genes. The vector and/or particle may be utilized for the purpose of transferring any nucleic acids into cells either in vitro or in vivo. Numerous forms of viral vectors are known in the art.

### Cells

Some embodiments of the various aspects described herein include a cell, e.g., a host cell. For example, the disclosure provides a cell comprising a polypeptide described herein or a polynucleotide encoding a polypeptide described herein. As used herein, the term “cell” refers to a single cell as well as to a population of (i.e., more than one) cells. As used herein, the cell can be a prokaryotic or eukaryotic cell. Exemplary cells include, but are not limited to, bacterial cells, yeast cells, plant cell, animal (including insect) or human cells.

In some embodiments of any one of the aspect, the cell is a host cell. The host cells can be employed in a method of producing a polypeptide described herein. Generally, the method comprises: culturing the host cell comprising a polynucleotide encoding a polypeptide described herein or a plasmid or vector comprising the polynucleotide under conditions such that the polypeptide is expressed; and optionally recovering the polypeptide from the culture medium. The polypeptide can be concentrated and purified by a variety of biochemical and chromatographic methods, including methods utilizing differences in size, charge, hydrophobicity, solubility, specific affinity, etc. between the polypeptide and other substances in the cell culture medium. In some embodiments of the various aspects described herein, the polypeptide is secreted from the host cells.

The polypeptide described herein can be produced as recombinant molecules in prokaryotic or eukaryotic host cells, such as bacteria, yeast, plant, animal (including insect) or human cell lines or in transgenic animals. Recombinant methods of producing a polypeptide through the introduction of a vector including nucleic acid encoding the polypeptide into a suitable host cell is well known in the art, such as is described in Sambrook et al., Molecular Cloning: A Laboratory Manual, 2d Ed, Vols 1 to 8, Cold Spring Harbor, NY (1989); M. W. Pennington and B. M. Dunn, Methods in Molecular Biology: Peptide Synthesis Protocols, Vol 35, Humana Press, Totawa, NJ (1994), contents of both of which are herein incorporated by reference.

The production of polypeptides at high levels in suitable host cells requires the assembly of the polynucleotides encoding such polypeptides into efficient transcriptional units together with suitable regulatory elements in a recombinant expression vector that can be propagated in various expression systems according to methods known to those skilled in the art. Efficient transcriptional regulatory elements could be derived from viruses having animal cells as their natural hosts or from the chromosomal DNA of animal cells. For example, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 can be used. In order to achieve stable high levels of mRNA, the transcriptional unit should contain in its 3′-proximal part a DNA region encoding a transcriptional termination-polyadenylation sequence. Generally, this sequence can be derived from the Simian Virus 40 early transcriptional region, the rabbit beta-globin gene, or the human tissue plasminogen activator gene.

The vector is transfected into a suitable host cell line for expression of the polypeptide. Examples of cell lines that can be used to prepare the polypeptide described herein include, but are not limited to monkey COS-cells, mouse L-cells, mouse C127-cells, hamster BHK-21 cells, human embryonic kidney 293 cells, and hamster CHO-cells.

The expression vector encoding the polypeptide can be introduced in several different ways. For instance, the expression vectors can be created from vectors based on different animal viruses. Examples of these are vectors based on baculovirus, vaccinia virus, adenovirus, and preferably bovine papilloma virus

The transcription units encoding the corresponding DNAs can also be introduced into animal cells together with another recombinant gene, which may function as a dominant selectable marker in these cells in order to facilitate the isolation of specific cell clones, which have integrated the recombinant DNA into their genome. Examples of this type of dominant selectable marker genes are Tn5 amino glycoside phosphotransferase, conferring resistance to geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the cDNA of the desired protein, or it can be encoded on a separate vector which is simultaneously introduced and integrated to the genome of the host cell, frequently resulting in a tight physical linkage between the different transcription units

Other types of selectable marker genes, which can be used together with the cDNA of the desired protein are based on various transcription units encoding dihydrofolate reductase (dhfr). After introduction of this type of gene into cells lacking endogenous dhfr-activity, preferentially CHO-cells (DUKX-B11, DG-44) it will enable these to grow in media lacking nucleosides. An example of such a medium is Ham's F12 without hypoxanthine, thymidin, and glycine. These dhfr-genes can be introduced together with the Kazal-type serine protease inhibitors' cDNA transcriptional units into CHO-cells of the above type, either linked on the same vector or on different vectors, thus creating dhfr-positive cell lines producing recombinant protein.

If the above cell lines are grown in the presence of the cytotoxic dhfr-inhibitor methotrexate, new cell lines resistant to methotrexate will emerge. These cell lines may produce recombinant protein at an increased rate due to the amplified number of linked dhfr and the desired protein's transcriptional units. When propagating these cell lines in increasing concentrations of methotrexate (1-10000 nM), new cell lines can be obtained which produce the desired protein at a very high rate.

The above cell lines producing the desired protein can be grown on a large scale, either in suspension culture or on various solid supports. Examples of these supports are micro carriers based on dextran or collagen matrices, or solid supports in the form of hollow fibers or various ceramic materials. When grown in cell suspension culture or on micro carriers the culture of the above cell lines can be performed either as a batch culture or as a perfusion culture with continuous production of conditioned medium over extended periods of time.

An example of such purification is the adsorption of the polypeptide to a monoclonal antibody or a binding peptide, which is immobilized on a solid support. After desorption, the protein can be further purified by a variety of chromatographic techniques based on the above properties.

Exemplary genera of yeast contemplated to be useful in the production of the polypeptide described herein as hosts are Pichia (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Genera include those selected from the group consisting of Saccharomyces, Schizosaccharomyces, Kluyveromyces, Pichia and Torulaspora. Examples of Saccharomyces spp. are S. cerevisiae, S. italicus and S. rouxii.

Suitable promoters for S. cerevisiae include those associated with the PGKI gene, GALI or GAL10 genes, CYCI, PHO5, TRPI, ADHI, ADH2, the genes for glyceral-dehyde-3-phosphate dehydrogenase, hexokinase, pyruvate decarboxylase, phos-phofructokinase, triose phosphate isomerase, phosphoglucose isomerase, glucokinase, alpha-mating factor pheromone, the PRBI, the GUT2, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (Maundrell K. 1990. Nmt1 of fission yeast. A highly transcribed gene completely repressed by thiamine. J. Biol. Chem. 265:10857-10864) and the glucose repressible jbpl gene promoter as described by Hoffman and Winston (Hoffman C S and Winston F. 1990. Isolation and characterization of mutants constitutive for expression of the fbp1 gene of Schizosaccharomyces pombe. Genetics 124:807-816).

The transcription termination signal may be the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is optionally used.

Exemplary expression systems for the production of the polypeptide described herein in bacteria include Bacillus subtilis, Bacillus brevis, Bacillus megaterium, Caulobacter crescentus, Escherichia coli BL21 and E. coli K12 and their derivatives. Convenient promoters include but are not limited to trc promoter, tac promoter, lac promoter, lambda phage promoter p_{L}, the L-arabinose inducible araBAD promoter, the L-rhamnose inducible rhaP promoter, and the anhydrotetracycline-inducible tetA promoter/operator.

In some embodiment, a polynucleotide encoding the polypeptide described herein can be fused to signal sequences which will direct the localization of a protein of the invention to particular compartments of a prokaryotic cell and/or direct the secretion of a protein of the invention from a prokaryotic cell. For example, in E. coli, one may wish to direct the expression of the protein to the periplasmic space. Examples of signal sequences or proteins (or fragments thereof) to which the proteins of the invention may be fused in order to direct the expression of the polypeptide to the periplasmic space of bacteria include, but are not limited to, the pelB signal sequence, the maltose binding protein signal sequence, the ompA signal sequence, the signal sequence of the periplasmic E. coli heat-labile enterotoxin B-subunit, and the signal sequence of alkaline phosphatase. Several vectors are commercially available for the construction of polypeptides which will direct the localization of a protein, such as the pMAL series of vectors (New England Biolabs).

Exemplary plant systems for expression of the polypeptide described herein include tobacco, potato, rice, maize, soybean, alfalfa, tomato, lettuce and legume (summarized by Ma J K C et al. 2003. The production of recombinant pharmaceutical proteins in plants. Nat. Rev. Genet. 4:794-805). Expression of recombinant proteins in plant systems may be directed by suitable regulatory elements to specific organs or tissues such as fruits, seeds, leaves or tubers. Alternatively, proteins may be secreted from the roots. Within the cell, proteins may be targeted to particular compartments, e.g. the endoplasmic reticulum, protein bodies or plastids. There the product may accumulate to higher levels or undergo particular forms of posttranslational modification.

Exemplary examples for large-scale transgenic expression systems (for review see Pollock D P. 1999. Transgenic milk as a method for the production of recombinant antibodies. J Immunol Methods 231:147-157) include rabbit (Chrenek P et al. 2007. Expression of recombinant human factor VIII in milk of several generations of transgenic rabbits. Transgenic Res. 2007 Jan. 31), goat (Lazaris A et al. 2006. Transgenesis using nuclear transfer in goats. Methods Mol Biol. 348:213-26), pig and cattle.

In some embodiments, the method comprises separating the expressed crude polypeptide from the host cell. When the host cell excretes polypeptide, the polypeptide can be separated from the host cell by harvesting the cell culture supernatant, which contains the excreted polypeptide. When the polypeptide is expressed in the periplasmic space of a host cell, e.g., a bacterial host cell, the cell can be lysed. Methods for lysing host cells are well known in the art. Exemplary methods of lysis include, but are not limited to, mechanical, chemical, thermal, enzymatic, or a combination thereof. In some embodiments, an osmotic shock procedure can be carried out to isolate the polypeptide expressed in the periplasmic space of a bacterial expression host.

One exemplary chemical method of lysis comprises adding a non-ionic surfactant to the cell culture or cell culture supernatant comprising the host cell. The non-ionic surfactant is added to a final concentration of at least about 0.05% (w/v, w/w or v/v) or higher and allowed to mix with the cell culture or cell culture supernatant for a sufficient period of time to lyse host cells present in the cell culture or cell culture supernatant. For example, the non-ionic surfactant is mixed with the cell culture or cell culture supernatant for a period of from about 15 minutes to about 2 hours. The mixing can be at ambient temperature or an elevated temperature. For example, the mixing with the non-ionic surfactant can be at a temperature from about 15° C. to about 37° C.

Exemplary non-ionic surfactants and classes of non-ionic surfactants for lysing host cells can include polyarylphenol polyethoxy ethers; polyalkylphenol polyethoxy ethers; polyglycol ether derivatives of saturated fatty acids; polyglycol ether derivatives of unsaturated fatty acids; polyglycol ether derivatives of aliphatic alcohols; polyglycol ether derivatives of cycloaliphatic alcohols; fatty acid esters of polyoxyethylene sorbitan; alkoxylated vegetable oils; alkoxylated acetylenic dials; polyalkoxylated alkylphenols; fatty acid alkoxylates; sorbitan alkoxylates; sorbitol esters; C₈ to C₂₂ alkyl or alkenyl polyglycosides; polyalkoxy styrylaryl ethers; alkylamine oxides; block copolymer ethers; polyalkoxylated fatty glyceride; polyalkylene glycol ethers; linear aliphatic or aromatic polyesters; organo silicones; polyaryl phenols; sorbitol ester alkoxylates; and mono- and diesters of ethylene glycol and mixtures thereof; ethoxylated tristyrylphenol; ethoxylated fatty alcohol; ethoxylated lauryl alcohol; ethoxylated castor oil; and ethoxylated nonylphenol; alkoxylated alcohols, amines or acids.

The cell culture or cell culture supernatant may comprise impurities, e.g., cellular material. Therefore, the method can comprise a post-lysis step of removing or reducing amount of impurities from the cell culture or cell culture supernatant. For example, the crude polypeptide can be further separated from cellular material, for example, via centrifugation and/or affinity purification.

### Kits

A polypeptide or polynucleotide described herein can be provided in a kit, e.g., as a component of a kit. For example, the kit includes (a) a polypeptide or polynucleotide described herein, and, optionally (b) informational material. The informational material can be descriptive, instructional, marketing or other material that relates to the methods described herein and/or the use of a polypeptide or polynucleotide described herein for the methods described herein. The informational material of the kits is not limited in its form. In some embodiments, the informational material can include information about production of the polypeptide or the polynucleotide encoding the polypeptide, their molecular weight, concentration, date of expiration, batch or production site information, and so forth. In some embodiments, the informational material relates to using the polypeptide or the polynucleotide to degrade or stabilize RNA. For example, the informational material relates to using the polypeptide or the polynucleotide to treat, prevent, or diagnosis of disorders and conditions.

In some embodiments, the informational material can include instructions to administer the polypeptide or the polynucleotide in a suitable manner to perform the methods described herein, e.g., in a suitable dose, dosage form, or mode of administration (e.g., a dose, dosage form, or mode of administration described herein). In another embodiment, the informational material can include instructions to administer the polypeptide or the polynucleotide to a suitable subject, e.g., a human, e.g., a human having, or at risk for, a disorder or condition needing treatment

The informational material of the kits is not limited in its form. In many cases, the informational material, e.g., instructions, is provided in print but can also be in other formats, such as computer readable material.

Components of the kit, e.g., the polypeptide and/or the polynucleotide can be provided in any form, e.g., liquid, dried or lyophilized form. It is preferred that the polypeptide or the polynucleotide be substantially pure and/or sterile. When the polypeptide or the polynucleotide is provided in a liquid solution, the liquid solution preferably is an aqueous solution, with a sterile aqueous solution being preferred. When the polypeptide or the polynucleotide is provided as a dried form, reconstitution generally is by the addition of a suitable solvent. The solvent, e.g., sterile water or buffer, can optionally be provided in the kit.

The kit can include one or more containers for the components of the kit. In some embodiments, the kit contains separate containers, dividers or compartments for the different components of the kit. For example, the polypeptide and/or the polynucleotide can be contained in a bottle, vial, or syringe, and the informational material can be contained association with the container. In other embodiments, the separate elements of the kit are contained within a single, undivided container. For example, the polypeptide and/or the polynucleotide is contained in a bottle, vial or syringe that has attached thereto the informational material in the form of a label. In some embodiments, the kit includes a plurality (e.g., a pack) of individual containers, each containing one or more-unit dosage forms of the polypeptide and/or the polynucleotide. For example, the kit includes a plurality of syringes, ampules, foil packets, or blister packs, each containing a single unit dose of the polypeptide and/or the polynucleotide. The containers of the kits can be air tight, waterproof (e.g., impermeable to changes in moisture or evaporation), and/or light-tight.

The kit optionally includes a device suitable for administration of the polypeptide and/or the polynucleotide, e.g., a syringe, inhalant, dropper (e.g., eye dropper), swab (e.g., a cotton swab or wooden swab), or any such delivery device. In some embodiments, the device is an implantable device that dispenses metered doses the polypeptide and/or the polynucleotide. The disclosure also features a method of providing a kit, e.g., by combining components described herein.

In some embodiments, the kit can further comprise additional components and/or reagents for practicing the methods described herein using the polypeptide and/or the polynucleotide described herein.

### Compositions

The polypeptides and/or the polynucleotides described herein can be formulated in compositions. For example, polypeptides and/or the polynucleotides described herein can be formulated into pharmaceutical compositions for therapeutic use. Accordingly, in another aspect, the invention provides a pharmaceutical composition comprising a polypeptide and/or polynucleotide described herein. Pharmaceutically acceptable compositions comprise a therapeutically-effective amount of one or more of the polypeptide and/or described herein, taken alone or formulated together with one or more pharmaceutically acceptable carriers (additives), excipient and/or diluents.

The pharmaceutical compositions can be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Delivery using subcutaneous or intravenous methods can be particularly advantageous.

The phrase “therapeutically-effective amount” as used herein means that amount of a compound, material, or composition comprising a conjugate described herein which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

As used herein, a “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical carriers include sterile aqueous solutions or dispersions and sterile powders for the extemporaneous preparation of sterile injectable solutions or dispersion. The use of such media and agents for pharmaceutically active substances is known in the art.

The formulations can conveniently be presented in unit dosage form and can be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

Pharmaceutical compositions for use with the methods described herein can be formulated in a conventional manner using one or more physiologically acceptable carriers or excipients. For example, a polypeptide, polynucleotide and/or oligonucleotide described herein can be formulated for administration by, for example, by aerosol, intravenous, oral or topical route. The compositions can be formulated for intralesional, intratumoral, intraperitoneal, subcutaneous, intramuscular or intravenous injection; infusion; liposome-mediated delivery; topical, intrathecal, gingival pocket, per rectum, intrabronchial, nasal, transmucosal, intestinal, oral, ocular or otic delivery.

Techniques and formulations generally can be found in Remmington's Pharmaceutical Sciences, Meade Publishing Co., Easton, PA. For systemic administration, injection is preferred, including intramuscular, intravenous, intraperitoneal, and subcutaneous. For injection, polypeptide, polynucleotide and/or oligonucleotide described herein can be formulated in liquid solutions, preferably in physiologically compatible buffers such as Hank's solution or Ringer's solution. In addition, the polypeptide, polynucleotide and/or oligonucleotide can be formulated in solid form and redissolved or suspended immediately prior to use. Lyophilized forms are also included.

For oral administration, the pharmaceutical composition can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they may be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., pharmaceutically acceptable oils, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner. For administration by inhalation, the compounds for use as described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The polypeptides and/or the polynucleotides can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

In addition to the formulations described previously, the polypeptides and/or the polynucleotides can also be formulated as a depot preparation. Such long acting formulations can be administered by implantation (for example subcutaneously or intramuscularly) or by intramuscular injection. Thus, for example, the polypeptides and/or the polynucleotides can be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Transmucosal administration can be through nasal sprays or using suppositories. For topical administration, polypeptides and/or the polynucleotides can be formulated into ointments, salves, gels, or creams as generally known in the art. A wash solution can be used locally to treat an injury or inflammation to accelerate healing.

The compositions can, if desired, be presented in a pack or dispenser device which can contain one or more-unit dosage forms containing the active ingredient. The pack can for example comprise metal or plastic foil, such as a blister pack. The pack or dispenser device can be accompanied by instructions for administration.

The polypeptides and/or the polynucleotides described herein can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the polypeptide, polynucleotide and/or oligonucleotide. The lipophilic material isolates the aqueous interior from an aqueous exterior, which typically does not include the polypeptide, polynucleotide and/or oligonucleotide, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include a polypeptide, polynucleotide or oligonucleotide described herein are delivered into the cell. In some cases, the liposomes are also specifically targeted, e.g., to direct the conjugate to particular cell types.

A liposome containing a polypeptide, polynucleotide or oligonucleotide described herein can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The polypeptide, polynucleotide or oligonucleotide is then added to the micelles that include the lipid component. After condensation, the detergent is removed, e.g., by dialysis, to yield a liposomal preparation.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide or oligonucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75:4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety).

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992) 269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

In some embodiments, cationic liposomes are used. Cationic liposomes possess the advantage of being able to fuse to the cell membrane.

Further advantages of liposomes include: liposomes obtained from natural phospholipids are biocompatible and biodegradable; liposomes can incorporate a wide range of water and lipid soluble drugs; liposomes can protect encapsulated polypeptides, polynucleotides or oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy) propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells.

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer the polypeptide, polynucleotide and/or oligonucleotide, into the skin. In some implementations, liposomes are used for delivering polypeptide, polynucleotide and/or oligonucleotide to epidermal cells and also to enhance the penetration of polypeptide, polynucleotide and/or oligonucleotide into dermal tissues, e.g., into skin. For example, the liposomes can be applied topically. Topical delivery of drugs formulated as liposomes to the skin has been documented (see, e.g., Weiner et al., Journal of Drug Targeting, 1992, vol. 2,405-410 and du Plessis et al., Antiviral Research, 18, 1992, 259-265; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y. and Huang, L., Proc. Natl. Acad. Sci. USA 84:7851-7855, 1987, which are incorporated by reference in their entirety).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin.

Liposomes that include a conjugate described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include polypeptide, polynucleotide and/or oligonucleotide can be delivered, for example, subcutaneously by infection. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Surfactants. Surfactants find wide application in formulations such as emulsions (including microemulsions) and liposomes (see above). A conjugate formulation can include a surfactant. In some embodiments, a conjugate described herein is formulated as an emulsion that includes a surfactant. The most common way of classifying and ranking the properties of the many different types of surfactants, both natural and synthetic, is by the use of the hydrophile/lipophile balance (HLB). The nature of the hydrophilic group provides the most useful means for categorizing the different surfactants used in formulations (Rieger, in “Pharmaceutical Dosage Forms,” Marcel Dekker, Inc., New York, NY, 1988, p. 285).

If the surfactant molecule is not ionized, it is classified as a nonionic surfactant. Nonionic surfactants find wide application in pharmaceutical products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in “Pharmaceutical Dosage Forms,” Marcel Dekker, Inc., New York, NY, 1988, p. 285).

Micelles and other Membranous Formulations. Formulations comprising a conjugate described herein can be provided as a micellar formulation. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the polypeptide, polynucleotide and/or oligonucleotide, an alkali metal C₈ to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains conjugate described herein and at least the alkali metal alkyl sulphate. The first micellar composition is then mixed with at least three micelle forming compounds to form a mixed micellar composition. In another method, the micellar composition is prepared by mixing conjugate described herein, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

Particles. In some embodiments, conjugate described herein can be incorporated into a particle, e.g., a microparticle. Microparticles can be produced by spray-drying, but may also be produced by other methods including lyophilization, evaporation, fluid bed drying, vacuum drying, or a combination of these techniques.

### Methods Using the Polypeptide

The polypeptides described herein can modulate RNAs. Accordingly, in one aspect, provided herein is a method for degrading or stabilizing RNA(s) to treat disease. The method comprises contacting the RNA with a polypeptide described herein. For example, to degrade aberrant RNA, a polypeptide comprising a phosphoserine mimetic at position 475 of human ZC3H14 amino acid sequence, e.g., SEQ ID NO: 1, or a corresponding position in a homologous or orthologous ZC3H14 protein can be used. In another example, to stabilize RNA(s) to treat disease, a polypeptide comprising a non-phosphoserine mimetic, e.g. a non-phosphorylatable residue at position 475 of human ZC3H14 amino acid sequence, e.g., SEQ ID NO: 1, or a corresponding position in a homologous or orthologous ZC3H14 protein can be used.

Exemplary aberrant RNAs that can be degraded to treat disease with the compositions and methods described herein include, but are not limited to prematurely terminated RNAs, e.g., RNAs that terminate at a cleavage and polyadenylation site prior to the dominant cleavage and polyadenylation site; RNAs with detained introns, e.g., introns that are recognized as introns by pre-spliceosome formation, but which the spliceosome stalls out on before splicing can occur; other protein coding RNAs that are polyadenylated. In some embodiments of any one of the aspects, the RNA comprises or contains a polyadenosine sequence.

Exemplary RNAs that can be stabilized to treat disease with the compositions and methods described herein include, but are not limited to any polyadenylated RNA. In some embodiments of any one of the aspects, the RNA comprises or contains a polyadenosine sequence.

It is noted that contacting with the RNA can be in a cell. Further, contacting with the RNA can be in vitro or in vivo. When the aberrant RNA is in a cell, the polypeptide or a nucleic acid encoding the polypeptide can be administered to the cell. As used herein, administering the polypeptide or polynucleotide to the cell means contacting the cell with the polypeptide or polynucleotide so that the polypeptide or polynucleotide is taken up by the cell. Generally, the cell can be contacted with the polypeptide or polynucleotide in a cell culture e.g., in vitro or ex vivo, or the polypeptide or polynucleotide can be administrated to a subject, e.g., in vivo. The term “contacting” or “contact” as used herein in connection with contacting a cell includes subjecting the cells to an appropriate culture media, which comprises a polypeptide or polynucleotide described herein. Where the cell is in vivo, “contacting” or “contact” includes administering the polypeptide or polynucleotide, e.g., in a pharmaceutical composition to a subject via an appropriate administration route such that the polypeptide or polynucleotide contacts the cell in vivo.

As described herein, the polypeptide or polynucleotide can be administered to a cell in vivo for degrading or stabilizing an RNA. Accordingly, in some embodiments, a therapeutically effective amount of a polypeptide or polynucleotide described herein can be administered to a subject for degrading or stabilizing an RNA. For example, a therapeutically effective amount of a polypeptide or polynucleotide can be administrated to a subject for treating a disease or disorder characterized by aberrant RNA. Thus, in yet another aspect, provided herein is a method of treating a disease or disorder characterized by an aberrant RNA. The method comprises administering a therapeutically effective amount of a polypeptide described herein or a polynucleotide encoding said polypeptide to a subject in need thereof.

Exemplary diseases and disorders associated with aberrant RNAs include, but are not limited to, cancers with mutant CDK13, mutant ZFC3H1, mutant ZC3H18, or another mutation that causes an increase in aberrant RNAs; developmental disorder with a mutation in CDK13, ZC3H14, or TRIP12; any disease with a protein coding RNA with a mutation in it, and any disease which is caused by an increase in detained introns, e.g., cancers that rely on detained introns for their proliferation such as malignant glioma and prostate cancer, e.g. aggressive prostate cancer. See, for example, Braun et al., Coordinated Splicing of Regulatory Detained Introns within Oncogenic Transcripts Creates an Exploitable Vulnerability in Malignant Glioma, Cancer Cell, 32 (4): 411-426.e11 (2017) and Zhang et al., Intron retention is a hallmark and spliceosome represents a therapeutic vulnerability in aggressive prostate cancer. Nat Commun 11, 2089 (2020), contents of both of which are incorporated herein by reference in their entirety. Accumulation of RNAs with detained introns also has been implicated in amyotrophic lateral sclerosis (ALS). See, for example, Humphrey et al., FUS ALS-causative mutations impair FUS autoregulation and splicing factor networks through intron retention. Nucleic Acids Res 48, 6889-6905 (2020), contents of which are incorporated herein by reference in their entirety. The data presented herein shown that expression of an exemplary polypeptide according to embodiments of the aspects described herein decrease the level of detained introns (FIG. 5) Accordingly, in some embodiments, the diseases and disorders associated with aberrant RNAs include ALS.

To therapeutically stabilize RNAs, a polypeptide comprising a non-phosphoserine mimetic, e.g., a non-phosphorylatable residue at position 475 of human ZC3H14 amino acid sequence, e.g., SEQ ID NO: 1, or a corresponding position in a homologous or orthologous ZC3H14 protein can be linked with a nucleic acid binding moiety, e.g., a catalytically dead CasRX (Konermann 2018). Without wishing to be bound by a theory, use of such polypeptide can stabilize the targeted RNA, thus raising levels of the targeted RNA. Stabilizing RNAs can be useful for treating diseases that are caused by destabilization of an important gene. Such diseases include, but are not limited to, IPEX syndrome (an autoimmune syndrome caused by destabilization of FOXP3), TP53 SNP at the 3′ end site that leads to a susceptibility for multiple cancers, alpha and beta Thalassaemias caused by a similar mechanism. In all of these disorders, the 3′ cleavage site is altered with a base change that causes destabilization of the transcript. The polypeptides provided herein can stabilize the affected transcript and provide therapeutic gene expression for these diseases. Other disease where increased RNA expression would be therapeutic could be treated by stabilizing the relevant RNA. For example, for any genetic disease caused by a heterozygous mutation, the wild type copy of the mRNA could be stabilized, rescuing protein levels and thus treating patients.

PAXT members are recurrently mutated in cancer and loss of nuclear RNA surveillance (clean up) is a tumor suppressive mechanism. Activation of this clean up pathway using a polypeptide comprising a phosphoserine mimetic at position 475 of human ZC3H14 amino acid sequence, e.g., SEQ ID NO: 1, or a corresponding position in a homologous or orthologous ZC3H14 protein, with or without a nucleic acid binding moiety, can be therapeutic for cancers that depend on deficient nuclear RNA surveillance. For example, a polypeptide comprising a phosphoserine mimetic at position 475 of human ZC3H14 amino acid sequence, e.g., SEQ ID NO: 1, or a corresponding position in a homologous or orthologous ZC3H14 protein, with or without a nucleic acid binding moiety, can be used to degrade aberrant RNAs. Degrading aberrant RNAs can be useful for treating diseases, such as cancers, that depend on deficient nuclear RNA surveillance.

Without wishing to be bound by a theory, degradation of aberrant RNAs can be used to treat several specific cancer genotypes:

CDK13 mutant tumors: It is known that loss of CDK13 is tumor suppressive via stabilization of aberrant RNAs. See, for example, doi: https://doi.org/10.1101/824193. Activating the degradation of aberrant RNAs using a polypeptide comprising a phosphoserine mimetic at position 475 of human ZC3H14 amino acid sequence, e.g., SEQ ID NO: 1, or a corresponding position in a homologous or orthologous ZC3H14 protein, with or without a nucleic acid binding moiety, can be therapeutic.

Other tumors deficient in nuclear RNA surveillance: PAXT adaptor proteins ZFC3H1 (Z1) and ZC3H18 (Z18) have recurrent mutations. Z1 K385Nfs*9 is detected in 23 different patient tumors and Z18 is mutated at R680 in 49 individual tumors from a broad spectrum of cancers. All recurrent mutations in Z1 and Z18 are predicted to cause a frame shift resulting in an early stop codon that truncates the protein. For both, the allelic fraction was <60% suggesting the mutation was heterozygous in patients' melanomas. Without wishing to be bound by a theory, these mutations work by interfering with the function of the WT protein because both copies are never mutated or lost. Activating the degradation of prematurely terminated RNAs using a polypeptide comprising a phosphoserine mimetic at position 475 of human ZC3H14 amino acid sequence, e.g., SEQ ID NO: 1, or a corresponding position in a homologous or orthologous ZC3H14 protein, with or without a nucleic acid binding moiety, can be therapeutic.

Detained intronic species: Degrading RNAs with detained introns is thought to be therapeutic in cancer. An exemplary polypeptide comprising the amino acid sequence of SEQ ID NO: 17 (ZC3H14 S475E) can destabilize RNAs with detained introns. Thus, the polypeptides, polynucleotides and methods described herein can be useful therapy for cancers reliant on detained introns.

Specific disease-causing RNAs: The polypeptides, polynucleotides and methods described herein can also be used for degrading specific RNAs. For example, a polypeptide comprising a phosphoserine mimetic at position 475 of human ZC3H14 amino acid sequence, e.g., SEQ ID NO: 1, or a corresponding position in a homologous or orthologous ZC3H14 protein can be linked with a nucleic acid binding moiety, e.g., a catalytically dead CasRX (Konermann 2018). Without wishing to be bound by a theory, use of the nucleic acid binding moiety can target and thereby degrade specific problematic RNAs.

Gain/loss of CPA: The polypeptides, polynucleotides and methods described herein can be useful for treating diseases associated with loss or gain of cleavage and polyadenylation sites (CPA). Some exemplary diseases associated with loss or gain of CPA are described in Gruber A J, Zavolan M. Alternative cleavage and polyadenylation in health and disease. Nat Rev Genet. 2019 October; 20 (10): 599-614, contents of which are herein incorporated by reference in their entirety. Exemplary loss of CPA diseases include, but are not limited to, immunodysregulation polyendocrinopathy enteropathy X-linked (IPEX syndrome—a poly(A) signal (PAS) alteration (AATAAA to AATGAA) in FOXP3 leads to IPEX syndrome), cancers (the presence of a single-nucleotide polymorphism (SNP) in a canonical PAS (AATAAA to AATACA) in TP53 is associated with impaired RNA 3′ end processing and an increased susceptibility to multiple cancers), α-Thalassaemia (poly(A) signal alterations (for example, AATAAA to AATAAG) in HBA2, which encodes haemoglobin subunit α2, lead to α-thalassaemia), β-Thalassaemia (poly(A) signal alterations (for example, AATAAA to AACAAA) in HBB, which encodes haemoglobin subunit-3, lead to β-thalassaemia), neonatal diabetes (a disruptive alteration in a poly(A) signal in INS, which encodes insulin, results in neonatal diabetes) and systemic lupus erythematosus (SLE—the presence of a SNP in a canonical poly(A) signal (AATAAA to AATAGA) in GIMAP5, which encodes GTPase IMAP family member 5, is associated with susceptibility to SLE). Exemplary gain of CPA diseases include, but are not limited to, SLE (the presence of a SNP in a proximal poly(A) signal (AATGAA to AATAAA) of IRF5, which encodes interferon regulatory factor 5, is associated with a high-risk haplotype for SLE), Wiskott-Aldrich syndrome (alterations in the locus of WAS, which encodes Wiskott-Aldrich syndrome protein, resulted in a patient expressing a novel 3′ isoform of WAS mRNA), Type I diabetes (the SNP in GIMAP5 that is associated with an increased susceptibility to SLE (see above) has also been associated with increased levels of islet cell autoantigen 2 autoantibodies in patients with type I diabetes), and thrombophilia (a CG-to-CA variant (G20210A) in F2, which encodes prothrombin, leads to an increase in the efficiency of RNA 3′ end processing and thus an increase in the expression of F2 mRNA and protein). For treating loss of disorders associated with loss of CPA, a polypeptide comprising a non-phosphoserine mimetic at position 475 of human ZC3H14 amino acid sequence, e.g., SEQ ID NO: 1, or a corresponding position in a homologous or orthologous ZC3H14 protein can be used to stabilize the RNA. Conversely, for treating disorders associated with gain of CPA, a polypeptide comprising a phosphoserine mimetic at position 475 of human ZC3H14 amino acid sequence, e.g., SEQ ID NO: 1, or a corresponding position in a homologous or orthologous ZC3H14 protein can be used to degrade the aberrant RNA.

Heterozygous diseases: It is known that heterozygous mutations in specific genes, e.g., NF1 and TP53 can cause disease such as Neurofibromatosis type 1 or Li-Fraumeni syndrome (LFS). In all of these disorders, a polypeptide comprising a phosphoserine mimetic at position 475 of human ZC3H14 amino acid sequence, e.g., SEQ ID NO: 1, or a corresponding position in a homologous or orthologous ZC3H14 protein, with or without a nucleic acid binding moiety can stabilize the wild type copy of the mRNA, rescuing protein levels and thus treating patients.

It is noted that the terms “administered” and “subjected” are used interchangeably in the context of treatment of a disease or disorder. In jurisdictions that forbid the patenting of methods that are practiced on the human body, the meaning of “administering” of a composition to a human subject shall be restricted to prescribing a controlled substance that a human subject will be administer to the subject by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of the polypeptide or polynucleotide described herein or a composition comprising the same into a subject by a method or route which results in at least partial localization of the composition at a desired site such that desired effect is produced. A polypeptide or polynucleotide described herein or a composition comprising the same can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, administration will generally be local rather than systemic. In some embodiments, administering is intravenous (IV) or intraperitoneal (IP) administration

The phrase “therapeutically effective amount” as used herein means that amount of a polypeptide or polynucleotide described herein described herein which is effective for producing some desired therapeutic effect in at least a sub-population of cells, e.g., activate the PAXT complex in a subject at a reasonable benefit/risk ratio applicable to any medical treatment. Thus, “therapeutically effective amount” means that amount which, when administered to a subject for treating pancreatitis, is sufficient to affect such treatment for pancreatitis.

Depending on the route of administration, effective doses can be calculated according to the body weight, body surface area, or organ size of the subject to be treated. Optimization of the appropriate dosages can readily be made by one skilled in the art in light of pharmacokinetic data observed in human clinical trials. Alternatively, or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound described herein is an amount sufficient to produce at least some desired therapeutic effect in a subject.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of use or administration utilized.

The effective dose can be estimated initially from cell culture assays. A dose can be formulated in animal models to achieve a circulating plasma concentration range that includes the IC₅₀ (i.e., the concentration of the therapeutic which achieves a half-maximal inhibition of symptoms) as determined in cell culture. Levels in plasma can be measured, for example, by high performance liquid chromatography. The effects of any particular dosage can be monitored by a suitable bioassay. The effective plasma concentration for a polypeptide described herein can be about 0.01 μM to about 10 μM, about 0.2 μM to about 5 μM, or about 0.8 to about 3 μM in a subject, such as a rat, dog, or human.

Generally, the compositions are administered so that a polypeptide described herein is used or given at a dose from 50 μg/kg to 1000 mg/kg; 1 μg/kg to 500 mg/kg; 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this disclosure, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

The polypeptide or polynucleotide described herein can be administered at once, or can be divided into a number of smaller doses to be administered at intervals of time. It is understood that the precise dosage and duration of treatment will be a function of the location of where the polypeptide or polynucleotide is administered, the carrier and other variables that can be determined empirically using known testing protocols or by extrapolation from in vivo or in vitro test data. It is to be noted that concentrations and dosage values can also vary with the age of the individual treated. It is to be further understood that for any particular subject, specific dosage regimens can need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The polypeptide or polynucleotide can be administered as a single bolus or multiple boluses, as a continuous infusion, or a combination thereof. For example, the polypeptide or polynucleotide can be administered as a single bolus initially, and then administered as a continuous infusion following the bolus. The rate of the infusion can be any rate sufficient to maintain effective polypeptide concentration, for example, to maintain effective plasma concentration. Some contemplated infusion rates include from 1 μg/kg/min to 100 mg/kg/min, or from 1 μg/kg/hr to 1000 mg/kg/hr. Rates of infusion can include 0.2 to 1.5 mg/kg/min, or more specifically 0.25 to 1 mg/kg/min, or even more specifically 0.25 to 0.5 mg/kg/min. It will be appreciated that the rate of infusion can be determined based upon the dose necessary to maintain effective plasma concentration and the rate of elimination of the compound, such that the compound is administered via infusion at a rate sufficient to safely maintain a sufficient effective plasma concentration of compound in the bloodstream.

It will be appreciated that methods of treatment of the present invention can be employed in combination with additional therapies. For example, a treatment according to the present disclosure can be co-administered with one or more desired therapeutics or medical procedures for treating pancreatitis.

The terms “co-administration” or the like, as used herein, are meant to encompass administration of the selected therapeutic agents to a single patient and are intended to include treatment regimens in which the agents are administered by the same or different route of administration or at the same or different time. The particular combination of therapies (therapeutics or procedures) to employ in such a combination regimen will take into account compatibility of the desired therapeutics and/or procedures and the desired therapeutic effect to be achieved.

Embodiments of the various aspects described herein can be described by the following numbered embodiments:

Embodiment 1: A polypeptide comprising an amino acid sequence having at least 85% identity to an amino acid sequence of wild-type ZC3H14 or a homologues or orthologues protein, and wherein the polypeptide comprises a mutation at position 475 of the wild-type ZC3H14 or a homologues or orthologues protein amino acid sequence.

Embodiment 2: The polypeptide of Embodiment 1, wherein the polypeptide comprises a phosphoserine mimetic at position 475 of the wild-type ZC3H14 amino acid sequence or a homologues or orthologues protein amino acid sequence.

Embodiment 3: The polypeptide of Embodiment 2, wherein said phosphoserine mimetic is an amino acid or a non-hydrolyzable phosphoserine mimetic.

Embodiment 4: The polypeptide of Embodiment 3, wherein said amino acid is aspartic acid or glutamic acid.

Embodiment 5: The polypeptide of Embodiment 3, wherein said non-hydrolyzable phosphoserine mimetic is L-2-amino-4 (diethylphosphono)-4,4-difluorobutanoic acid.

Embodiment 6: The polypeptide of Embodiment 1, wherein the polypeptide comprises a non-phosphoserine mimetic, e.g., a non-phosphorylatable residue at position 475 of the wild-type ZC3H14 amino acid sequence or a homologues or orthologues protein amino acid sequence.

Embodiment 7: The polypeptide of Embodiment 6, wherein the non-phosphoserine mimetic, e.g., the non-phosphorylatable residue is an amino acid.

Embodiment 8: The polypeptide of Embodiment 7, wherein the non-phosphoserine mimetic, e.g., the non-phosphorylatable residue an amino acid selected from the group consisting of alanine, valine, leucine, isoleucine, methionine, phenylalanine, tryptophan, cysteine, glycine, proline, and selenocystine.

Embodiment 9: The polypeptide of Embodiment 8 wherein the non-phosphoserine mimetic, e.g., the non-phosphorylatable residue is alanine.

Embodiment 10: The polypeptide of Embodiment 1, wherein the polypeptide comprises a S->D or S->A or S->E mutation at position 475 of the wild-type ZC3H14 amino acid sequence.

Embodiment 11: The polypeptide of any one of Embodiments 1-10, wherein the wild-type ZC3H14 is a mammalian ZC3H14.

Embodiment 12: The polypeptide of any one of Embodiments 1-11, wherein the wild-type ZC3H14 is a human ZC3H14.

Embodiment 13: The polypeptide of Embodiment 1, wherein the polypeptide comprises an amino acid sequence having at least 85% identity to the amino acid sequence of SEQ ID NO: 16, SEQ ID NO: 17, or SEQ ID NO: 18.

Embodiment 14: The polypeptide of any one Embodiment 1-13, wherein the polypeptide further comprises a nucleic acid binding moiety linked to the polypeptide.

Embodiment 15: The polypeptide of Embodiment 14, wherein the nucleic acid binding moiety comprises a nucleic acid binding domain of a nucleic acid binding protein.

Embodiment 16: The polypeptide of Embodiment 15, wherein the nucleic acid binding protein is selected from the group consisting of clustered regularly interspaced short palindromic repeats (CRISPR)-associated (Cas) proteins, zinc finger nucleases (ZFNs), transcription activator-like effector-based nucleases (TALENs), and Argonaute proteins.

Embodiment 17: The polypeptide of Embodiment 16, wherein the CRISPR/Cas protein is catalytically-dead CasRX.

Embodiment 18: The polypeptide of Embodiment 14-17, wherein the nucleic acid binding moiety lacks nuclease activity.

Embodiment 19: A composition comprising a polypeptide of any one of Embodiments 1-18

Embodiment 20: A cell comprising a polypeptide of any one of Embodiments 1-18.

Embodiment 21: A kit comprising a polypeptide of any one of Embodiments 1-18.

Embodiment 22: A polynucleotide encoding a polypeptide of any one of Embodiments 1-18.

Embodiment 23: The polynucleotide of Embodiment 22, wherein the polynucleotide is comprised in a vector.

Embodiment 24: A cell comprising a polynucleotide of Embodiment 22 or 23.

Embodiment 25: A composition comprising a polynucleotide of Embodiment 22 or 23.

Embodiment 26: A kit comprising a polynucleotide of Embodiment 22 or 23.

Embodiment 27: A method for degrading an aberrant RNA, the method comprising contacting an aberrant RNA with a polypeptide of any one of Embodiments 1-18, wherein the polypeptide comprises a phosphoserine mimetic, e.g., a non-phosphorylatable residue at position S475 of the wild-type ZC3H14 amino acid.

Embodiment 28: A method for stabilizing an RNA, the method comprising contacting an RNA with a polypeptide of any one of Embodiments 1-18, wherein the polypeptide comprises a phosphoserine mimetic, e.g., a non-phosphorylatable residue at position S475 of the wild-type ZC3H14 amino acid.

Embodiment 29: The method of Embodiment 27 or 28, wherein the aberrant RNA is selected from the group consisting of prematurely terminated RNAs, RNAs with detained introns and ptRNAs.

Embodiment 30: The method of any one of Embodiments 27-29, wherein the aberrant RNA comprises a polyadenosine sequence.

Embodiment 31: The method of any one of Embodiments 27-30, wherein the aberrant RNA is in cell.

Embodiment 32: The method of Embodiment 31, wherein the method comprising administering a polynucleotide encoding the polypeptide to the cell.

Embodiment 33: The method of any one of Embodiments 27-32, wherein said contacting is in vitro.

Embodiment 34: The method of any one of Embodiments 27-32, wherein said contacting is in vivo.

Embodiment 35: The method of Embodiment 34, wherein said contacting in vivo is in a mammal.

Embodiment 36: The method of Embodiment 35, wherein the mammal is a human.

Embodiment 37: The method of Embodiment 35 or 36, wherein said contacting is in a subject and wherein the subject has a disease or disorder characterized by the aberrant RNA.

Embodiment 38: A method of treating a disease or disorder characterized by an aberrant RNA, the method comprising administering a polypeptide of any one of Embodiments 1-18 or a polynucleotide encoding the polypeptide to a subject in need thereof.

Embodiment 39: The method of Embodiment 37 or 38, wherein the disease or disorder characterized by an aberrant RNA is selected from the group consisting of: cancers with mutant CDK13, mutant ZFC3H1, mutant ZC3H18, or another mutation that causes an increase in aberrant RNAs; developmental disorder with a mutation in CDK13, ZC3H14, or TRIP12; a disease with a protein coding RNA with a mutation in it; a disease which is caused by an increase in detained introns; a disease caued by gain or loss of CPA; and any combinations thereof.

Embodiment 40: The method of Embodiment 39, wherein the disease or disorder characterized by an aberrant RNA is a cancer with mutant CDK13.

Embodiment 41: The method of Embodiment 39, wherein the disease or disorder characterized by an aberrant RNA is a disease caused by an increase in detained introns.

Embodiment 42: The method of Embodiment 41, wherein the disease caused by an increase in detained introns is malignant glioma, prostate cancer, amyotrophic lateral sclerosis (ALS).

### Some Selected Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a disease or disorder. A subject can be male or female.

A subject can be one who has been previously diagnosed with or identified as suffering from or having a condition in need of treatment. Alternatively, a subject can also be one who has not been previously diagnosed. A “subject in need” of testing for a particular condition can be a subject having that condition, diagnosed as having that condition, or at risk of developing that condition.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The terms “wild-type” or “wt” or “native” as used herein is meant an amino acid sequence or a nucleotide sequence that is found in nature, including allelic variations. A wild-type protein, polypeptide, antibody, immunoglobulin, IgG, polynucleotide, DNA, RNA, and the like has an amino acid sequence or a nucleotide sequence that has not been intentionally modified.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

The term “amino acid substitution” refers to the replacement of at least one existing amino acid residue in a predetermined or native amino acid sequence with a different “replacement” amino acid. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

The term “amino acid insertion” refers to the insertion of one or more additional amino acids into a predetermined or native amino acid sequence. The insertion can be one, two, three, four, five, or up to twenty amino acid residues.

The term “amino acid deletion” refers to removal of at least one amino acid from a predetermined or native amino acid sequence. The deletion can be one, two, three, four, five, or up to twenty amino acid residues.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity according to the assays described herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous or orthologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

The term “nucleic acid” refers to a deoxyribonucleotide or ribonucleotide and polymers thereof in either single strand or double strand form. The term “nucleic acid” is used interchangeably with gene, nucleotide, polynucleotide, cDNA, DNA, and mRNA. The polynucleotides can be in the form of RNA or DNA. Polynucleotides in the form of DNA, cDNA, genomic DNA, nucleic acid analogs, and synthetic DNA are within the scope of the present invention. Unless specifically limited the term encompasses nucleic acids containing known analogues of natural nucleotides that have similar binding propertied as the natural nucleic acid. Unless specifically limited, a particular nucleotide sequence also encompasses conservatively modified variants thereof (for example, those containing degenerate codon substitutions) and complementary sequences as well as the as well as the sequences specifically described.

The polynucleotides can be composed of any polyribonucleotide or polydeoxyribonucleotide, which can be unmodified RNA or DNA or modified RNA or DNA. For example, polynucleotides can be composed of single or double stranded regions, mixed single or double stranded regions. In addition, the polynucleotides can be triple stranded regions containing RNA or DNA or both RNA and DNA. Modified polynucleotides include modified bases, such as tritylated bases or unusual bases such as inosine. A variety of modification can be made to RNA and DNA, thus polynucleotide includes chemically, enzymactically, or metabolically modified forms.

The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

A variant DNA or amino acid sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of the various aspects described herein, a polypeptide, nucleic acid, or cell as described herein can be engineered. As used herein, “engineered” refers to the aspect of having been manipulated by the hand of man. For example, a polynucleotide is considered to be “engineered” when at least one aspect of the polynucleotide, e.g., its sequence, has been manipulated by the hand of man to differ from the aspect as it exists in nature.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The term “statistically significant” or “significantly” refers to statistical significance and generally means a two standard deviations (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%. In some embodiments of the various aspects described herein, the term “about” when used in connection with percentages can mean±5%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

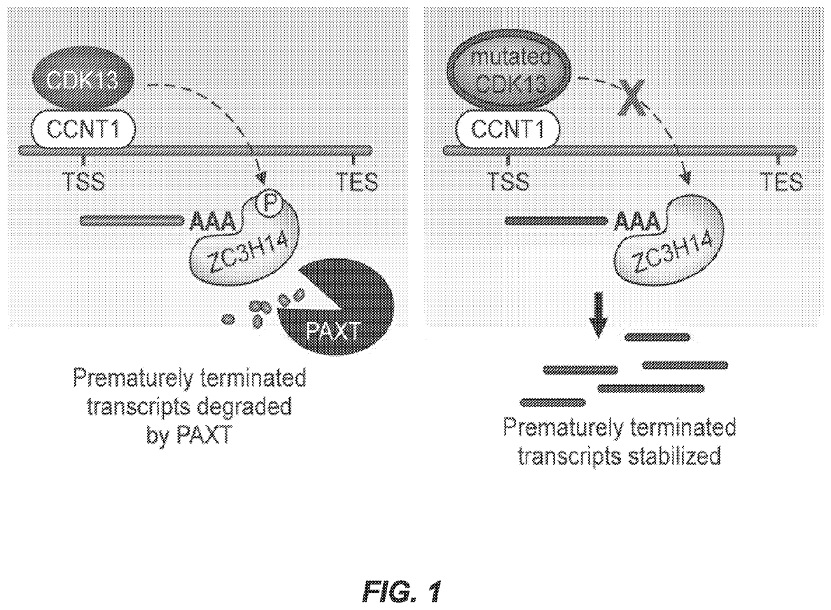

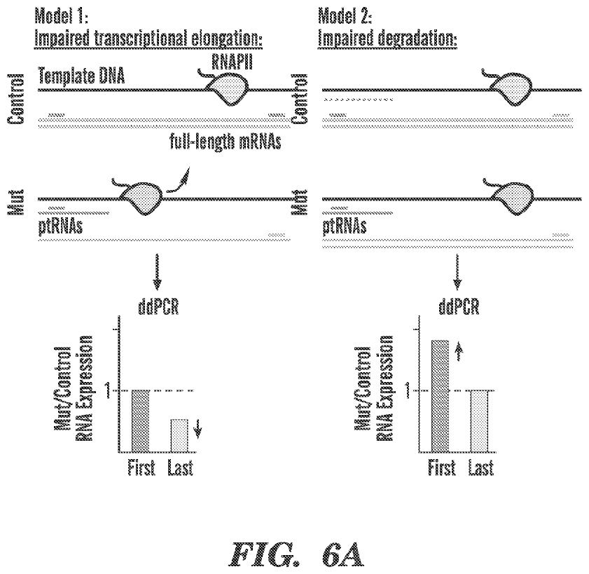

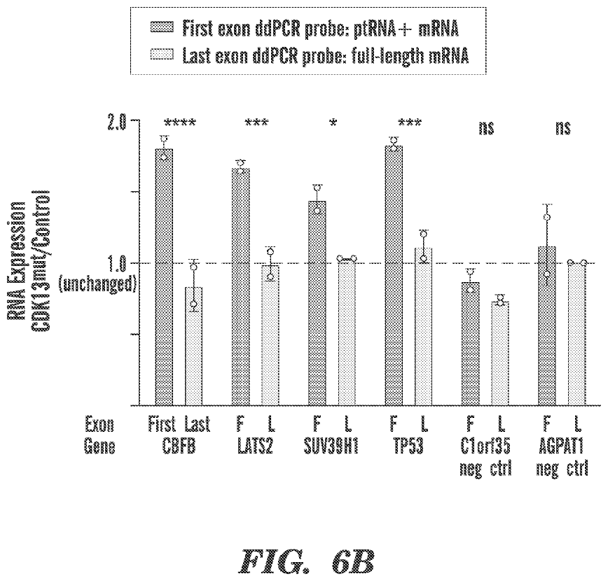

Data summary discussion: We then have found that ZC3H14 S475 phosphoryation is necessary and sufficient for activation of the PAXT nuclear exosome degradation complex to degrade aberrant transcripts in the nucleus (FIG. 1). We identified that CDK13 normally activated the turnover of prematurely terminated RNAs and when CDK13 activity is lost, these RNA are stabilized (instead of being made at higher levels) via digital droplet PCR (FIG. 6).

We found that CDK13 bound to both PABPN1 and ZC3H14 via IP-MS. Both of ZC3H14 and PABPN1 were recently shown to interact with the Poly A tail eXosome Targeting (PAXT) complex (Meola et al., 2016), which is responsible for targeting ptRNAs for degradation (Ogami et al., 2017). As ptRNAs accumulate in CDK13^{mut} melanoma via a post-transcriptional mechanism (FIGS. 6A-6B), and ptRNAs with a canonical cleavage and polyadenylation signal have been shown to be degraded by the PAXT complex (Wu et al., 2020), we hypothesized that CDK13 normally works to activate PAXT activity. Further, CDK13's physical interaction with two proteins involved in the PAXT nuclear RNA surveillance complex led us to hypothesize that CDK13 normally activates PAXT activity. We further hypothesized that loss of CDK13 kinase activity would fail to activate PAXT, leading to accumulated ptRNAs in melanoma.

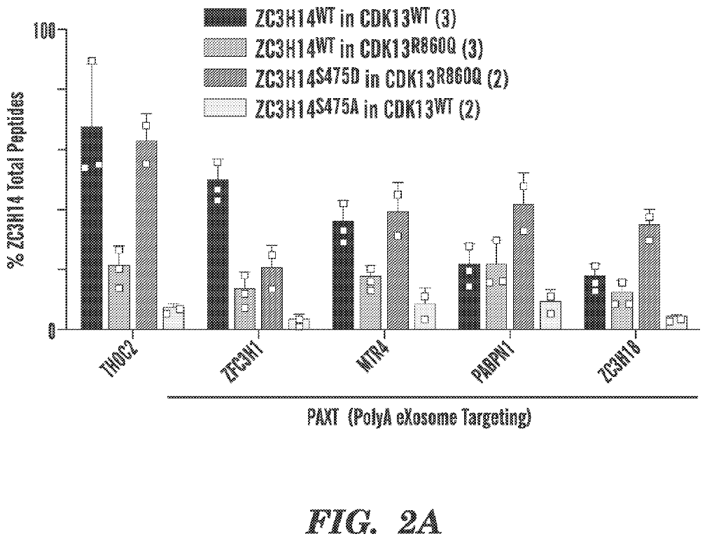

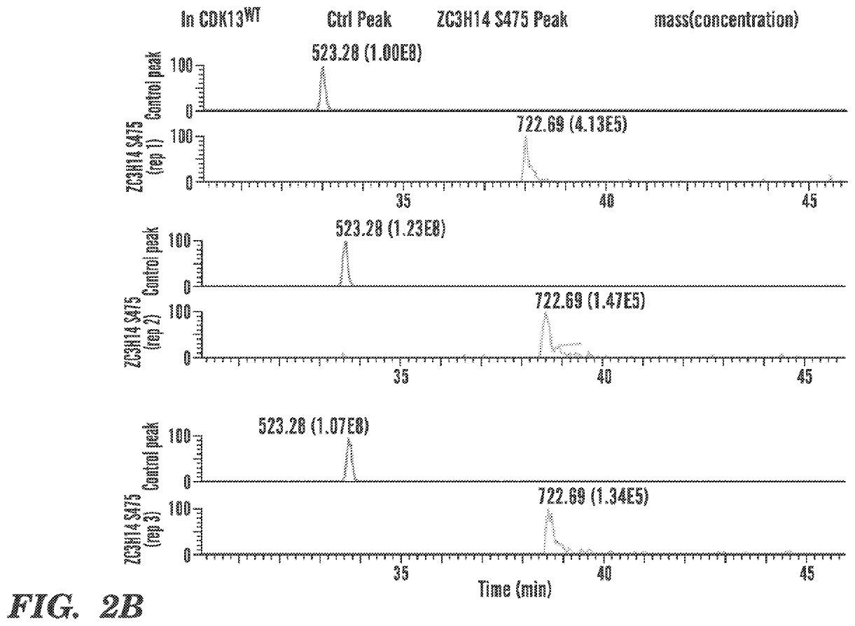

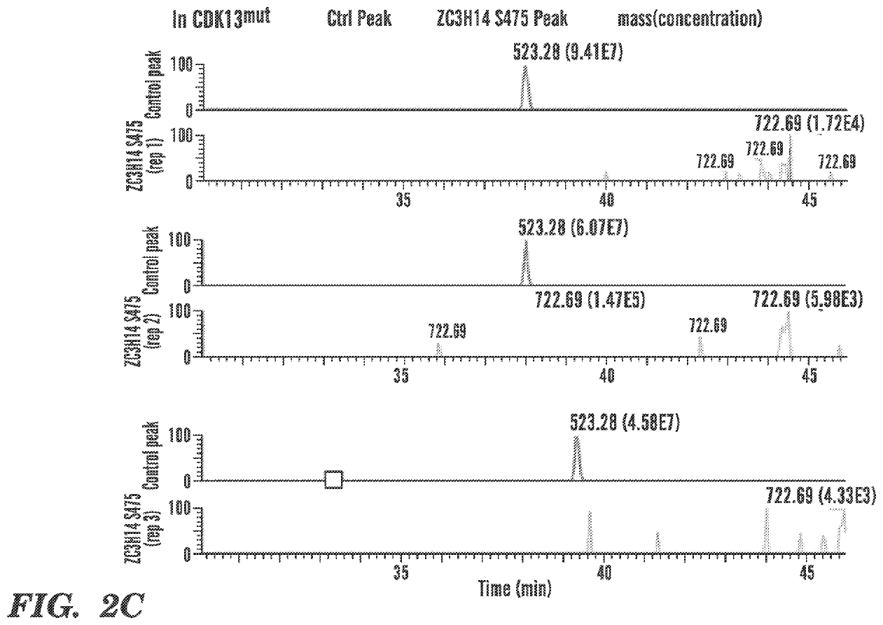

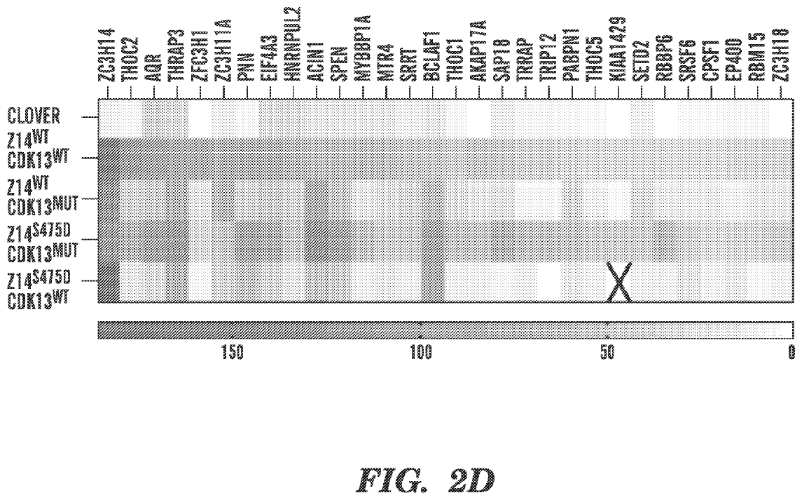

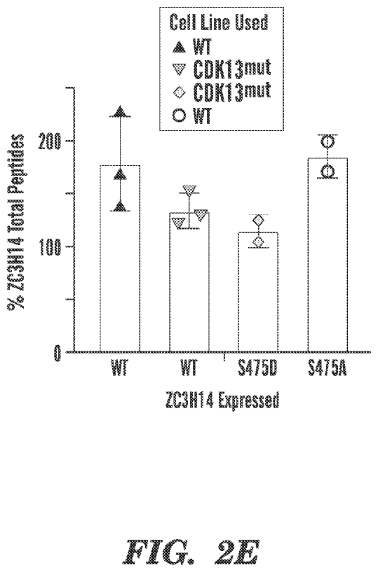

As PABPN1 and ZC3H14 are genetically antagonistic (Pak et al., 2011; Rha et al., 2017) and because we observed more ZC3H14 in our CDK13^{WT} IP, we chose to characterize ZC3H14 phosphorylation and binding partners in the presence and absence of CDK13 kinase activity. The nuclear isoform of ZC3H14 and a control protein were tagged, transiently expressed, and IPed from CDK13^{WT} and CDK13^{mut} human melanoma cells (FIGS. 2A-2E). IP-MS proteins were considered for downstream analyses if >3 peptides were identified per IP replicate and if the average total peptides were >3× enriched over the control IP. Reassuringly, ZC3H14 was the most abundant protein identified in all ZC3H14 IPs (FIGS. 2D and 2E). ZC3H14 had four phosphorylation sites when IPed from CDK13^{WT} cells, while in CDK13^{mut} cells, ZC3H14 lost phosphorylation only at S475 (FIGS. 2B and 2C). These data indicate that CDK13 kinase activity is required for ZC3H14 S475 phosphorylation.

We next looked at ZC3H14's binding partners in the presence and absence of CDK13 kinase activity. When ZC3H14 was IPed from CDK13^{mut} cells, ZC3H14 had fewer binding partners (FIG. 2D) despite an unchanged ZC3H14 peptide number (FIG. 2E). The ratio of total measured peptides to bait ZC3H14 was calculated. Differential binding was calculated between CDK13^{WT} and CDK13^{mut} conditions (t-test p-value<0.05), which identified 18 proteins that required intact CDK13 kinase activity for binding ZC3H14. The three most abundant proteins that required CDK13 kinase activity for binding to ZC3H14 were THOC2, ZFC3H1, and MTR4 (FIG. 2A, black vs. blue bars) (THOC2 q<0.0001, ZFC3H1 q<0.0001, and MTR4 q=0.018). THOC2 functions in RNA export and has been reported previously to bind ZC3H14 (Morris and Corbett, 2018), ZFC3H1 is a linker between the PAXT and the nuclear RNA degradation machinery, and MTR4 is a helicase required for nuclear RNA degradation (Meola et al., 2016). We also identified that ZC3H14 bound PAXT proteins PABPN1 and ZC3H18, however CDK13 kinase activity was not required for these proteins to bind ZC3H14 (FIG. 2A). These data show that ZC3H14 binds to multiple PAXT members and that CDK13 kinase activity is required for ZC3H14 binding to THOC2 and two key PAXT components-ZFC3H1, and MTR4.

To test whether the ZC3H14 S475 phosphorylation was sufficient to recruit PAXT binding, phospho-mimetic ZC3H14^{S475D} was tagged, transiently expressed, and IPed from cells lacking CDK13 kinase activity. ZC3H14^{S475D} was sufficient to rescue binding of PAXT components to ZC3H14 (FIG. 2A red bars; FIG. 2D, fourth row), even in cells lacking CDK13 kinase activity. To test whether ZC3H14 S475 was necessary for PAXT recruitment, phospho-dead ZC3H14^{S475A} was IPed from CDK13^{WT} cells. ZC3H14^{S475A} failed to recruit PAXT components (FIG. 2A, green bars; FIG. 2D, last row), even in cells with intact CDK13 kinase activity. Importantly, the amount of ZC3H14 IPed was unchanged (FIG. 2D, left; FIG. 2E). Together, these data show that ZC3H14 S475 phosphorylation is necessary and sufficient to recruit PAXT proteins to ZC3H14.

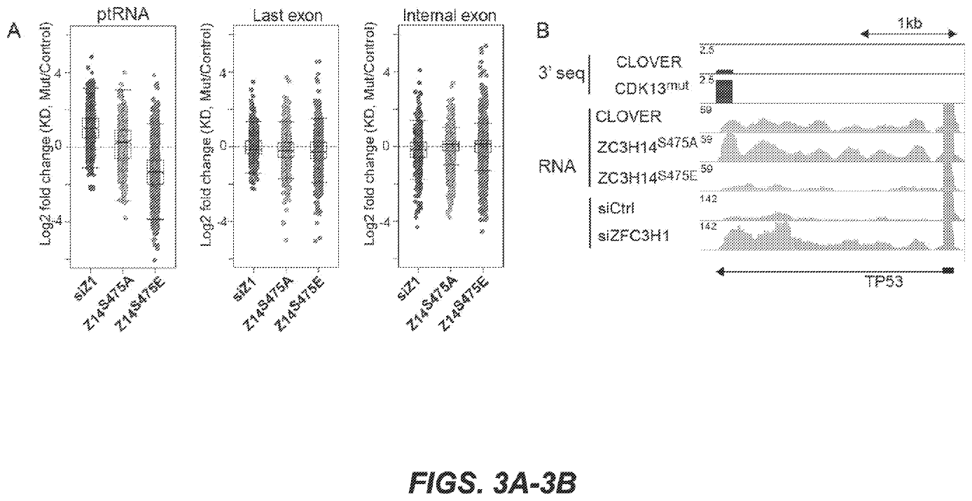

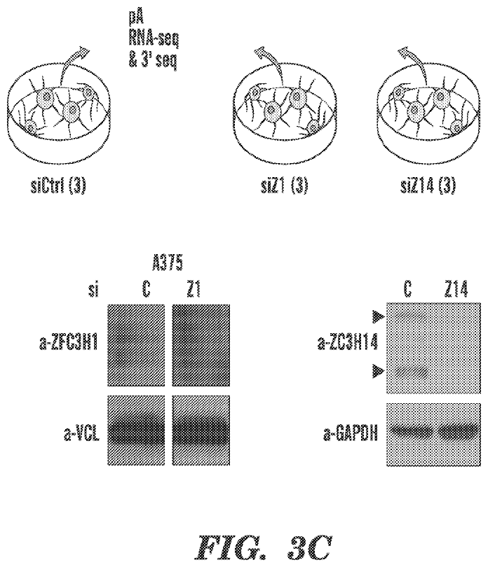

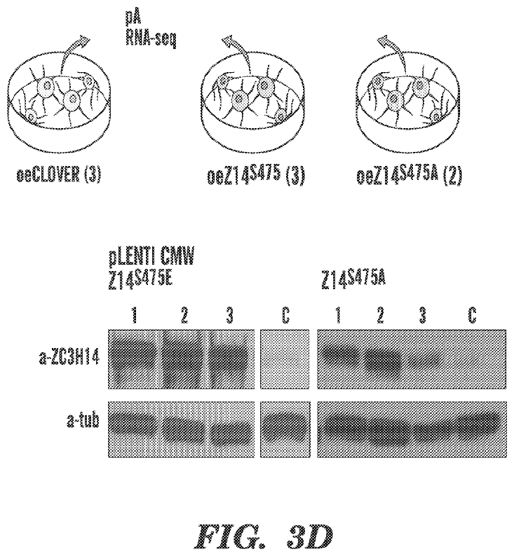

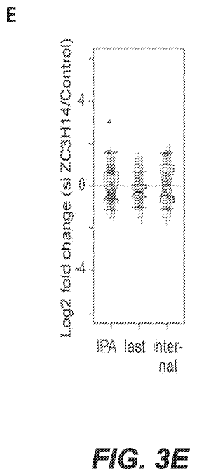

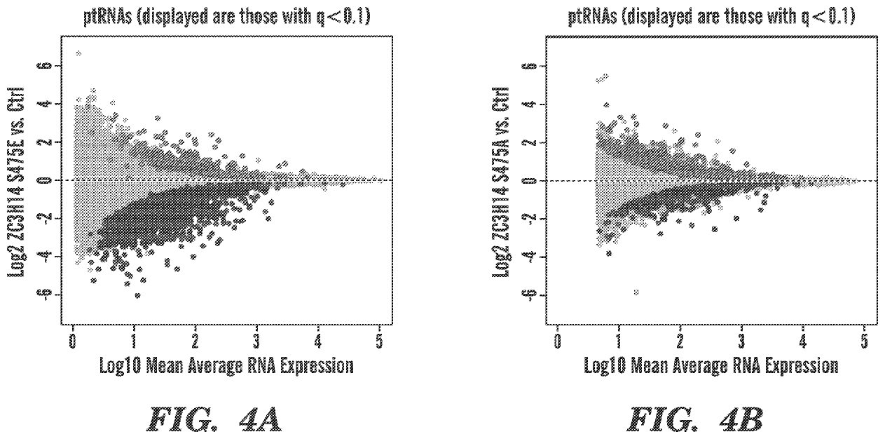

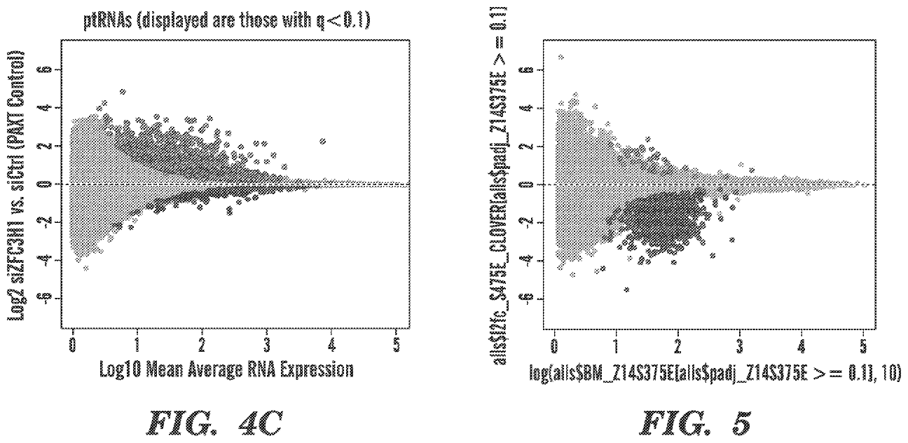

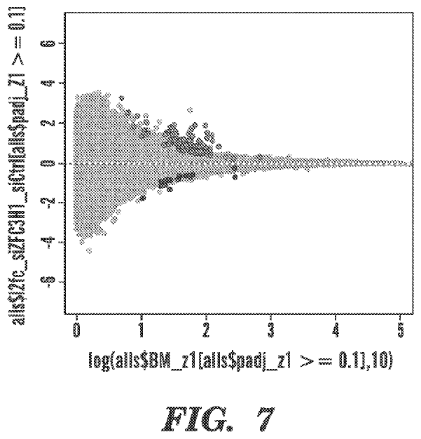

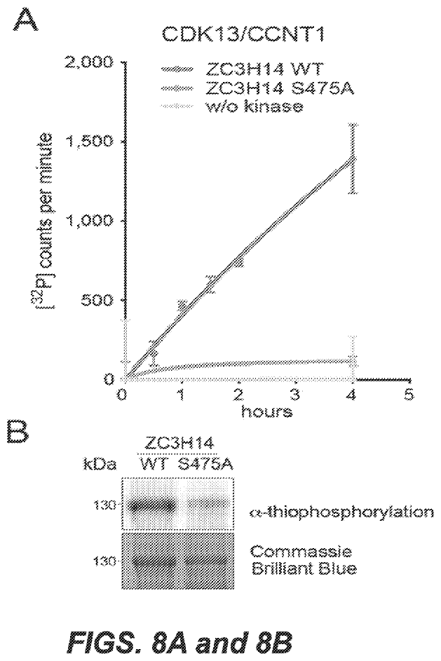

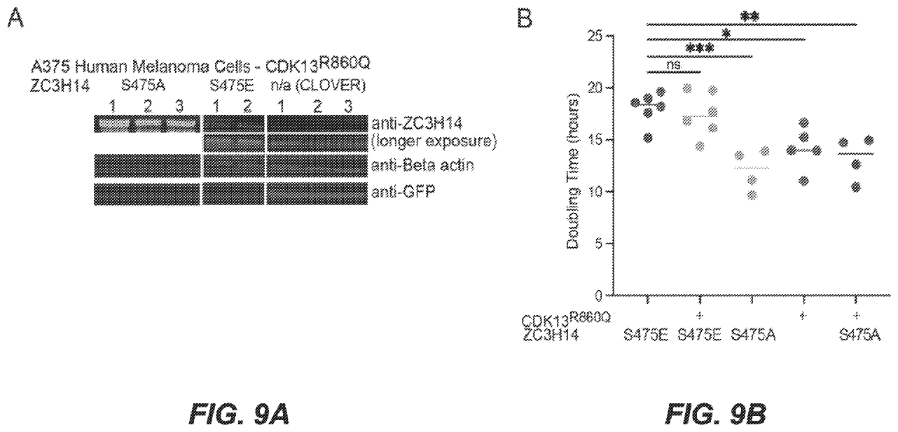

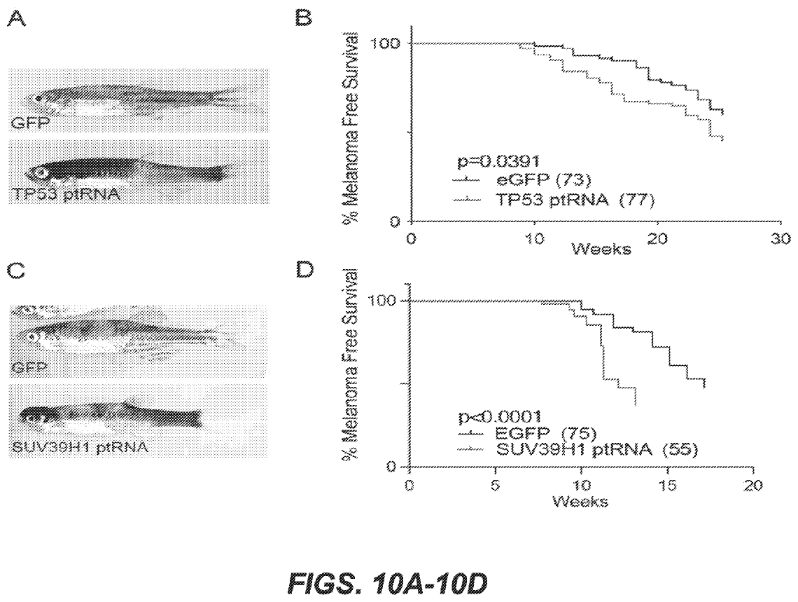

To test whether ZC3H14 S475 phosphorylation is also necessary and sufficient to activate ptRNA degradation, a two-pronged approach was undertaken. First, short interfering RNAs were used to decrease levels of ZFC3H1 (n=3) and ZC3H14 (n=3), and differential RNA expression was assessed using 3′seq and RNA-seq (FIG. 3C) as compared to a scrambled control (n=3). Second, stable human melanoma cell lines expressing phospho-dead ZC3H14^{S475A} (n=2), phospho-mimetic ZC3H14^{S475E} (n=3) or a control protein (n=3) were subjected to RNA-seq (FIG. 3D). ZC3H14^{S475E} was used because we were unable to make stable lines with ZC3H14^{S475D} Knockdown of ZFC3H1 and ZC3H14 and expression of ZC3H14^{S475E} or ZC3H14^{S475A} were confirmed via immunoblot (FIGS. 3C and 3D). 3′ sequencing from siZFC3H1, siZC3H14, siControl, and prior CDK13^{mut} and control samples were used to build an expression map for ptRNA and dominant isoforms in human melanoma cells. This ptRNA isoform expression map was used calculate differential RNA expression using the DEX-seq platform in cells with knocked down ZFC3H1, knocked down ZC3H14, ZC3H14^{S475A}-expression, or ZC3H14^{S475E}-expression as compared to the appropriate controls. A boxplot of significantly changed RNAs (p<0.1) from ZFC3H1 knockdown in human melanoma cells demonstrated an increase in ptRNA isoforms, but stable last exons and constitutive internal exons as expected (FIG. 3A, red; FIG. 4C). Knockdown of ZC3H14 visualized in the same manner showed very few significant expression changes (FIG. 3E), which may be due to activity of residual protein or redundancy with another protein. Expression of ZC3H14^{S475A} showed a modest but significant increase in ptRNA expression, while last and constitutive internal exons remained unchanged (FIG. 3A, yellow; FIG. 4B). Expression of ZC3H14^{S475E} caused a significant decrease in ptRNAs while not affecting last or internal exons (FIG. 3A, blue; FIG. 4A). We also found that activation of PAXT via ZC3H14^{S475E} caused degradation of RNAs with detained introns (FIG. 5) and that blocking of PAXT via knockdown of ZFC3H1 caused an increase in detained intron RNA species (FIG. 7), thus allowing us to propose that using ZC3H14 phosphomimetic could be therapeutic for cancers are dependent on RNAs with detained introns. For example, the TP53 ptRNA, which is upregulated in CDK13^{mut} cells, was also upregulated in ZFC3H1 knockdown, and upon phospho-dead ZC3H14^{S475A}-expression, consistent with loss of PAXT nuclear RNA degradation. In contrast phospho-mimetic ZC3H14^{S475E} caused this TP53 ptRNA to be expressed at lower levels, consistent with hyper-activation of PAXT RNA degradation (FIG. 3B). Together these data show that ZC3H14 S475 phosphorylation is necessary and sufficient to activate PAXT to degrade ptRNAs. Our data demonstrate that upon loss of CDK13 function, the PAXT complex is not efficiently recruited to ptRNAs, resulting in their stabilization, export, and translation into truncated polypeptides.

IP-MS: ZC3H14 WT, ZC3H14^{S475D}, or ZC3H14^{S475A} were cloned via the gateway system into pcDNA3.2 C-terminal V5 tag destination vector (ThermoFisher 12489019). Constructs were transiently transfected using Lipofectamine®3000 (Thermofisher) in 15 cm2 plate with independent replicates into either A375s or CDK13^{R860Q} expressing A375s. 48 hours after transfection, nuclei were isolated (Thermofisher 78833) and lysed per protocol. Anti-V5 (Clone V5-10, V8012 Sigma) was conjugated to protein G beads (Thermofisher 10004D). IPed proteins were eluted and submitted for mass spectrometry using the Taplin Mass Spectrometry Facility at Harvard University. Proteins from CDK13 experiment were included in analysis if all replicates had >1 peptide and the CDK13^{WT} IP identified >3× signal over the control IP. For the ZC3H14 IP, proteins were filtered if they had at least 4 peptides in each ZC3H14 replicate and were >3× enriched over control IP (all control IPs were combined). Statistics were done using multiple t-tests assuming similar scatter using Prism software.

ZC3H14 phosphorylation sites were reported if they were identified in all three biologic replicates with either 1) modification score >10 or 2)>2 peptides calling the same site. Spectra are shown for the S475 phosphorylation which is missing in the CDK13^{R860Q} condition.

RNA-Seq: For siZFC3H1 (n=3), siZC3H14 (n=3), and siControl (n=3) samples, equal cell numbers were gathered and ERCC was also spiked in proportional to cell number. For ZC3H14^{S475E} (n=3), ZC3H14^{S475A} (n=2), and CLOVER (n=2)-expressing samples, equal RNA amounts were used. RNA-isolation and genomic DNA removal was completed using a column method as above for zebrafish. All samples were poly A selected (NEB, E7490) and NEBNext Ultra II Directional RNA Library Prep Kit for Illumina (E7760S) was used. Samples were sequenced on a HiSeq with paired end sequencing. 3′ Sequencing: 3′ seq was completed as above for zebrafish melanoma samples for A375 human melanoma cells expressing: 1) pLENTI CMV CDK13^{R860Q} (n=3), 2) pLENTI CMV CLOVER (n=3), 3) siZFC3H1 (n=3), 4) siZC3H14 (n=3), and 5) siControl (n=3).

RNA-seq/3′ Sequencing Data Processing: Processing was performed as described above for zebrafish (see 3′ sequencing data processing, identification of 3′ cleavage sites) except data were mapped to hg38. As in the zebrafish analysis, each set of replicates from the different conditions was first processed separately, and then combined to produce a single set of non-redundant cleavage sites. In order to enable quantification of differential polyadenylation site usage in the ZC3H14^{S475E/A} mutants as well as TCGA samples for which we have poly(A) selected RNA-seq but not 3′ end sequencing data, we performed the quantifications in a different manner. Rather than directly counting reads at each 3′ cleavage site as before, we used the genomic locations of the combined cleavage site map combined with the RNA-seq splice junctions to produce a gtf annotating global alternative polyadenylation sites. In the case of intronic polyadenylation sites, the gtf contains an exon for each intronic site that spans from the 5′ end of the closest upstream exon to the 3′ end of the cleavage site cluster. In the case of distal poly(A) sites, the exon spans from the 5′ end of the last exon in which the site is contained, to the 3′ end of the cleavage cluster. DEXseq uses read density over these entire exon regions, rather than the 3′end sequencing counts, to quantify polyadenylation site differential usage and can thus be performed using standard poly(A)-selected RNA-seq data. The DEXseq script dexseq_count.py was used to obtain read counts for each sample using the DEXseq-formatted gtf. DEXseq was then run on each pairwise knockdown or mutant overexpression with its corresponding control to obtain log 2-fold differences and statistical significance at each ptRNA, last exon, and internal constitutive exon. RNA-seq bam files were visualized using IGV (FIG. 3B).

### 3′ Sequencing and RNA Sequencing in ZC3H14 Mutant and ZFC3H1 Knockdown Cell Lines

3′ sequencing and RNA sequencing: Knockdown of ZFC3H1 (Dharmacon, L-020839-02-0005) and a control knockdown (Dharmacon, D-001810-01-5) were completed on A375 CLOVER cells. Knockdown was confirmed via immunoblot (Novus, NB100-68267). ZC3H14 expressing cell lines were generated via lentiviral transduction and stable antibiotic selection with the following coding sequences ZC3H14, ZC3H14 S475A, ZC3H14 S475E. RNA was saved in RNA later and extracted using a Qiagen kit with a genomic DNA removal column (Qiagen, 741134). Poly-A selection was undertaken (NEB, E7490). RNA was prepped for 3′ sequencing using the QuantSeq 3′ mRNA-Seq Library Prep Kit REV for Illumina (016.24) or for RNA-seq was done using NEBNext Ultra II Directional RNA Library Prep Kit for Illumina (E7760S).

Custom sequencing primers provided by Lexogen were used for 3′ sequencing (standard Illumina sequencing primers and PhiX were avoided per protocol).

3′ sequencing and RNA sequencing data processing: Paired-end reads from QuantSeq 3′ end sequencing were mapped using STAR aligner version 2.7.2a (Dobin et al., 2013). Genome for human GRCz11 were downloaded from GENCODE and genome indexes were generated using the-sjbGTFfile flag. After mapping, a custom Python script was used to filter reads from each bam file based on the following parameters: 1) Proper mate pairing; 2) orientation of the putative 3′ cleavage end corresponding to the direction of transcription for the gene to which reads mapped; 3) concordant mapping to known chromosomes; 4) no soft clippings; 5) skipped regions must be longer than 70 nucleotides. Following initial filtering of reads, bedtools cluster tool version 2.26.0 (Quinlan and Hall, 2010) and custom Python scripts were used to generate read clusters by grouping any 3′ end coordinates that fell within 40 nucleotides of one another into a single cluster. Clustering was performed on each individual replicate separately. Recognized clusters from knockdown of ZFC3H1, control knockdown, or the previous 3′ sequencing from CDK13 mutants and their controls were used to generate a GTF file that recognizes intronic polyadenylation site terminated transcripts as RNA isoforms along with dominant expressed isoforms. This GTF was then used to quantify the RNA-seq from above using DEX-seq.

ddPCR: Equal cell numbers were collected from CDK13^{R860Q} and CLOVER-expressing biologic duplicate lines. Genomic DNA was removed and RNA was isolated via columns (Qiagen 74134). PolyA selection was done (E7490 protocol) and cDNA was made (Thermofisher, 18080400). CDK13 expression was verified using ddPCR. ddPCR was completed with FAM probes designed to the first (or second) and last exon of 4 genes with ptRNAs and 2 genes without ptRNAs. Thresholding was completed manually. CDK13^{mut} RNA concentration was divided by control CLOVER RNA concentration for each target. Normalization was completed against a control gene.

In vitro kinase assays: For in vitro kinase assays, human wildtype Cdk13 (694-1039) was co-purified together with human wildtype Cyclin Tl (residues 1-272) T149E mutant and CAKI from S. cerevisiae in Sf9 insect cells and purified as described previously (9). Human ZC3H14 full-length (residues 1-736) was cloned into a pET28a-MBP vector and expressed in E. coli BL21 pLys cells. Cell pellets were resuspended in resuspension buffer (50 mM Tris pH 8.5, 150 mM KCl, 0.02% NP40, 5 mM MgCl2, 1 mM EDTA, 10% glycerol), disrupted by sonication, followed by centrifugation at 50,000 g for 30 min at 10° C. Pellets were resuspended in resuspension buffer and subjected to ammonium sulfate precipitation by step-wise addition of 10%, 30% and 50% ammonium sulfate, followed by centrifugation at 50,000×g for 15 min at 10° C. after each step. After the last centrifugation step, the pellet was resuspended in resuspension buffer without salt, which was added after complete resuspension to a concentration of 150 mM NaCl. Samples were subjected to MBP affinity and Size exclusion chromatography using a Superdex S200 column (Cytiva) in SEC buffer (20 mM Hepes pH 8.2, 150 mM NaCl, 1 mM TCEP). For radioactive in vitro kinase assays 0.5 μM kinase was incubated with 25 mM substrate and 0.2 mM ATP containing 0.45 mCi [32P]-γ-ATP/mL (Perkin Elmer) in kinase buffer (150 mM HEPES pH 7.6, 34 mM KCl, 7 mM MgCl2, 2.5 mM dithiothreitol, 5 mM β-glycerol phosphate). Reactions were incubated for 0, 30, 60, 90, 120 and 240 min at 30° C. and 300 rpm, and stopped by addition of EDTA to a final concentration of 50 mM. Samples were spotted onto Amersham Protran nitrocellulose membrane (GE Healthcare), followed by three washing steps for 5 min each with PBS. Counts per minutes were determined in a Beckman Liquid Scintillation Counter (Beckman-Coulter) for 1 min. Measurements were performed in triplicates and represented as mean with standard deviation (SD). For Western blots 0.5 M Cdk13/Cyclin Tl was incubated with 25 mM MBP-ZC3H14 full-length wildtype and S475A mutant with 2 mM ATP-y-S(Jena Bioscience) in kinase buffer (150 mM HEPES pH 7.6, 34 mM KCl, 7 mM MgCl2, 2.5 mM dithiothreitol, 5 mM β-glycerol phosphate). Reactions were incubated for 240 min at 30° C. and 300 rpm, and stopped by addition of EDTA to a final concentration of 50 mM. Samples were alkylated with 2.5 mM para-nitrobenzylmesylate (PNBM; abcam) for 1 hour at room temperature and were then mixed with SDS sample buffer. After SDS-PAGE with 2 μl sample loaded and blotting onto Amersham Protran nitrocellulose membrane (Cytiva), membranes were blocked with 5% Bovine Serum Albumin in 0.1% Tween 20 in PBS (PBS-T) and incubated with a primary antibody diluted 1:5,000 in PBS-T directed against a thiophosphate ester (Allen et al., 2007; abcam #ab92570). A goat-anti rabbit immunoglobulin (IgG) horseradisch peroxidase (HRP)-coupled antibody (Invitrogen) was used as secondary antibody diluted 1:5,000 in PBST. Membranes were analyzed using ECL-solution and a CCD camera ChemiDoc XRS+ system (BioRad). A second SDS gel stained with Coomassie Brilliant Blue was used as a loading control.

Cell line generation and cell counts: A375 human melanoma cells were identity-verified via STR analysis and then used for transient transfections for IP-MS or for stable line generation. Mycoplasma testing was done within one week of every experiment using human cell lines (Lonza, Mycoalert PLUS, LT07-710). All cell lines were mycoplasma negative. Cells were grown in DMEM supplemented with 10% FBS, penicillin/streptomycin or selection antibiotics, and L-glutamine. Stable lines were made in biologic triplicate using lentiviral expression under a CMV promoter and maintained in selection antibiotics. Cell counts were done at 24, 48, 72, and 96 hours. Cell doubling times were calculated in exponential growth phase. Antibiotics were removed for cell line growth experiments.

Immunoblots: For ZFC3H1 westerns, protein was run on a 3-8% tris acetate gel and wet gel transfer was done. For other proteins, 10% tris glycine gels were used with semi-dry transfer. The following antibodies were used: anti-VCL (loading control) (Sigma HPA 002131, 1:2500), anti-GFP (Santa Cruz sc-9996, 1:1000), anti-ZFC3H1 (Novus, NB100-68267), anti-ZC3H14 (Sigma, HPA 049798), Tubulin (ab6160), or GAPDH (Invitrogen PA1-987). Rabbit or mouse secondary HRP antibodies were incubated for 1 hr at RT (CST 7074S or 7076S, 1:2000).

Expression of ptRNAs in a zebrafish melanoma model: Animal studies were approved by Boston Children's Hospital Institutional Animal Care and Use Committee (Protocol 17-10-3530R). Experiments were performed as published (10) (11). Briefly, p53^{-/-}; mitfa:BRAF^{V600E}; mitfa−/− one-cell embryos were injected with either 20 ng/uL control or experimental DNA along with tol2 in vitro transcribed RNA for integration. Two ptRNAs that were verified using ddPCR were cloned and expressed in melanocytes. In all experiments, DNA which overexpresses a gene-of-interest is marked with an mitfa mini gene which rescues Mitfa, allowing cell autonomous melanocyte genetics. In overexpression experiments, control vectors expressed EGFP. Embryos were sorted for melanocyte rescue at 5 days post fertilization (dpf) unless otherwise notes. 20 zebrafish were raised per tank to control for density effects. Zebrafish were scored for the emergence of raised melanoma lesions. Melanoma-free survival curves and Log-rank tests were generated in Prism. Photos were taken of zebrafish at 7 weeks.

### REFERENCES

1. Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
2. Fasken, M. B., Corbett, A. H., and Stewart, M. (2019). Structure-function relationships in the Nab2 polyadenosine-RNA binding Zn finger protein family. Protein Sci 28, 513-523.
3. Gruber, A. J., and Zavolan, M. (2019). Alternative cleavage and polyadenylation in health and disease. Nat Rev Genet 20, 599-614.
4. Konermann, S., Lotfy, P., Brideau, N. J., Oki, J., Shokhirev, M. N., and Hsu, P. D. (2018). Transcriptome Engineering with RNA-Targeting Type VI-D CRISPR Effectors. Cell 173, 665-676 e614.
5. Meola, N., Domanski, M., Karadoulama, E., Chen, Y., Gentil, C., Pultz, D., Vitting-Seerup, K., Lykke-Andersen, S., Andersen, J. S., Sandelin, A., et al. (2016). Identification of a Nuclear Exosome Decay Pathway for Processed Transcripts. Mol Cell 64, 520-533.
6. Morris, K. J., and Corbett, A. H. (2018). The polyadenosine RNA-binding protein ZC3H14 interacts with the THO complex and coordinately regulates the processing of neuronal transcripts. Nucleic Acids Res 46, 6561-6575.
7. Ogami, K., Richard, P., Chen, Y., Hoque, M., Li, W., Moresco, J. J., Yates, J. R., 3rd, Tian, B., and Manley, J. L. (2017). An Mtr4/ZFC3H1 complex facilitates turnover of unstable nuclear RNAs to prevent their cytoplasmic transport and global translational repression. Genes Dev 31, 1257-1271.
8. Pak, C., Garshasbi, M., Kahrizi, K., Gross, C., Apponi, L. H., Noto, J. J., Kelly, S. M., Leung, S. W., Tzschach, A., Behjati, F., et al. (2011). Mutation of the conserved polyadenosine RNA binding protein, ZC3H14/dNab2, impairs neural function in Drosophila and humans. Proc Natl Acad Sci USA 108, 12390-12395.
9. A. K. Greifenberg et al., Structural and Functional Analysis of the Cdk13/Cyclin K Complex. Cell Rep 14, 320-331 (2016).
10. C. J. Ceol et al., The histone methyltransferase SETDB1 is recurrently amplified in melanoma and accelerates its onset. Nature 471, 513-517 (2011).
11. C. K. Kaufman et al., A zebrafish melanoma model reveals emergence of neural crest identity during melanoma initiation. Science 351, aad2197 (2016).
12. Rha, J., Jones, S. K., Fidler, J., Banerjee, A., Leung, S. W., Morris, K. J., Wong, J. C., Inglis, G. A. S., Shapiro, L., Deng, Q., et al. (2017). The RNA-binding protein, ZC3H14, is required for proper poly(A) tail length control, expression of synaptic proteins, and brain function in mice. Hum Mol Genet 26, 3663-3681.
13. Wu, G., Schmid, M., Rib, L., Polak, P., Meola, N., Sandelin, A., and Jensen, T. H. (2020). A Two-Layered Targeting Mechanism Underlies Nuclear RNA Sorting by the Human Exosome. Cell Rep 30, 2387-2401 e2385.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A polypeptide comprising an amino acid sequence having at least 85% identity to an amino acid sequence of wild-type ZC3H14 or a homologues or orthologues protein, and wherein the polypeptide comprises a phosphoserine mimetic mutation at position 475 of the wild-type ZC3H14 or a homologues or orthologues protein amino acid sequence, wherein said phosphoserine mimetic is an amino acid or a non-hydrolyzable phosphoserine mimetic.

2. (canceled)

3. (canceled)

4. The polypeptide of claim 1, wherein said amino acid is or glutamic acid.

5. The polypeptide of claim 1, wherein said non-hydrolyzable phosphoserine mimetic is L-2-amino-4 (diethylphosphono)-4,4-difluorobutanoic acid.

6. A polypeptide comprising an amino acid sequence having at least 85% identity to an amino acid sequence of wild-type ZC3H14 or a homologues or orthologues protein, and wherein the polypeptide comprises a mutation at position 475 of the wild-type ZC3H14 or a homologues or orthologues protein amino acid sequence, wherein the polypeptide comprises a non-phosphorylatable residue at position 475 of the wild-type ZC3H14 amino acid sequence or a homologues or orthologues protein amino acid sequence.

7. The polypeptide of claim 6, wherein the non-phosphorylatable residue is an amino acid.

8. The polypeptide of claim 7, wherein the non-phosphorylatable residue is an amino acid selected from the group consisting of alanine, valine, leucine, isoleucine, methionine, phenylalanine, tryptophan, cysteine, glycine, proline, and selenocystine.

9. The polypeptide of claim 8, wherein the non-phosphorylatable residue moiety is alanine.

10. (canceled)

11. The polypeptide of claim 1, wherein the wild-type ZC3H14 is a mammalian ZC3H14.

12. The polypeptide of claim 1, wherein the wild-type ZC3H14 is a human ZC3H14.

13. The polypeptide of claim 1, wherein the polypeptide comprises an amino acid sequence having at least 85% identity to the amino acid sequence:
(ZC3H14S475E, SEQ ID NO: 17)
MEIGTEISRKIRSAIKGKLQELGAYVDEELPDYIMVIVIVANKKSQDQMT
EDLSLFLGNNTIRFTVWLHGVLDKLRSVTTEPSSLKSSDTNIFDSNVPSN
KSNFSRGDERRHEAAVPPLAIPSARPEKRDSRVSTSSQESKTTNVRQTYD
DGAATRLMSTVKPLREPAPSEDVIDIKPEPDDLIDEDLNFVQENPLSQKK
PTVTLTYGSSRPSIEIYRPPASRNADSGVHLNRLQFQQQQNSIHAAKQLD
MQSSWVYETGRLCEPEVLNSLEETYSPFFRNNSEKNISMFDENFRKRKLP
VVSSVVKVKKFNHDGEEEEEDDDYGSRTGSISSSVSVPAKPERRPSLPPS
KQANKNLILKAISEAQESVTKTTNYSTVPQKQ1LPVAPRTRTSQEELLAE
VVQGQSRTPRISPPIKEEETKGDSVEKNQGTQQRQLLSRLQIDPVMAETL
QMSQDYYDMESMVHADTRSFILKKPKLEEEVVVAPNQESGMKTADSLRVL
SGHLMQTRDLVQPDKPASPKF1VTLDGVPSPPGYMSDQEEDMCFEGMKPV
NQTAASNKGLRGLLHPQQLHLLSRQLEDPNGSFSNAEMSELSVAQKPEKL
LERCKYWPACKNGDECAYHHPISPCKAFPNCKFAEKCLFVHPNCKYDAKC
TKPDCP11HVSRRIPVLSPKPAVAPPAPPSSSQLCRYFPACKKMECPFYH
PKHCRFNTQCTRPDCTFYHPTINVPPRHALKWIRPQTSE;
or
(ZC3H14S475A, SEQ ID NO: 18)
MEIGTEISRKIRSAIKGKLQELGAYVDEELPDYIMVIVIVANKKSQDQMT
EDLSLILGNNTIRFTVWLHGVLDKLRSVTTEPSSLKSSDTNIFDSNVPSN
KSNFSRGDERRHEAAVPPLAIPSARPEKRDSRVSTSSQESKTTNVRQTYD
DGAATRLMSTVKPLREPAPSEDVIDIKPEPDDLIDEDLNFVQENPLSQKK
PTVTLTYGSSRPSIEIYRPPASRNADSGVHLNRLQFQQQQNSIHAAKQLD
MQSSWVYETGRLCEPEVLNSLEETYSPFFRNNSEKVISMEDENFRKRKLP
VVSSVVI(VKKFNHDGEEEEEDDDYGSRTGSISSSVSVPAKPERRPSLPP
SKQANKNLILKAISEAQESVTKTTNYSTVPQKQTLPVAPRTRTSQEELLA
EVVQGQSRTPRISPPIKEEETKGDSVEKNQGTQQRQLLSRLQ1DPVMAET
LQMSQDYYDMESMVHADTRSFILKKPKLAEEVVVAPNQESGMKTADSLRV
LSGIALMQTRDLVQPDKPASPKFIVTLDGVPSPPGYMSDQEEDMCFEG1V
IKPVNQTAASNKGLRGLLHPQQLHLLSRQLEDPNGSFSNAEMSELSVAQK
PEKLLERCKYWPACKNGDECAYHEPISPCKAFPNCKFAEKCLFVHPNCKY
DAKCTKPDCPFIHVSRRIPVLSPKPAVAPPAPPSSSQLCRYFPACKKMEC
PFYHPKHCRFNTQCTRPDCTFYHPTINVPPRHALKWIRPQTSE

14. The polypeptide of claim 1, wherein the polypeptide further comprises a nucleic acid binding moiety linked to the polypeptide.

15. The polypeptide of claim 14, wherein the nucleic acid binding moiety comprises a nucleic acid binding domain of a nucleic acid binding protein; or wherein the nucleic acid binding moiety lacks nuclease activity.

16. The polypeptide of claim 15, wherein the nucleic acid binding protein is selected from the group consisting of clustered regularly interspaced short palindromic repeats (CRISPR)-associated (Cas) proteins, zinc finger nucleases (ZFNs), transcription activator-like effector-based nucleases (TALENs), and Argonaute proteins.

17. The polypeptide of claim 16, wherein the CRISPR/Cas protein is catalytically-dead CasRX.

18. (canceled)

19. (canceled)

20. (canceled)

21. (canceled)

22. A polynucleotide encoding a polypeptide of claim 1.

23. (canceled)

24. (canceled)

25. (canceled)

26. (canceled)

27. A method for degrading an aberrant RNA, the method comprising contacting an aberrant RNA with the polypeptide of claim 1, wherein the polypeptide comprises a phosphoserine mimetic at position S475 of the wild-type ZC3H14 amino acid.

28. A method for stabilizing an aberrant RNA, the method comprising contacting an aberrant RNA with a polypeptide of claim 1, wherein the polypeptide comprises a non-phosphoserine mimetic which is not capable of phosphorylation at position S475 of the wild-type ZC3H14 amino acid.

29. The method of claim 27, wherein the aberrant RNA is selected from the group consisting of prematurely terminated RNAs, RNAs with detained introns, or other polyadenylated RNAs; or wherein the aberrant RNA comprises a polyadenosine sequence; or wherein the aberrant RNA is in cell.

30. (canceled)

31. (canceled)

32. The method of claim 29, wherein the polypeptide is encoded by a polynucleotide.

33. The method of claim 27, wherein said contacting is in vitro.

34. The method of claim 27, wherein said contacting is in vivo.

35. The method of claim 34, wherein said contacting in vivo is in a mammal.

36. The method of claim 35, wherein the mammal is a human.

37. The method of claim 35, wherein the human has a disease or disorder characterized by the aberrant RNA.

38. A method for stabilizing RNA(s) to correct a disease or disorder, the method comprising contacting an RNA with the polypeptide of claim 1, wherein the polypeptide comprises a non-phosphoserine mimetic which is not capable of phosphorylation at position S475 of the wild-type ZC3H14 amino acid.

39. A method of treating a disease or disorder characterized by an aberrant RNA, the method comprising administering the polypeptide of claim 1 or a polynucleotide encoding the polypeptide to a subject in need thereof.

40. The method of claim 39, wherein the disease or disorder characterized by an aberrant RNA is selected from the group consisting of: cancers with mutant CDK13, mutant ZFC3H1, mutant ZC3H18, or another mutation that causes an increase in aberrant RNAs; melanoma; developmental disorder with a mutation in CDK13, ZC3H14, or TRIP12; a disease with a protein coding RNA with a mutation in it; a disease which is caused by an increase in detained introns, malignant glioma, prostate cancer, amyotrophic lateral sclerosis (ALS), a disease caused by gain or loss of CPA; and any combinations thereof.

41. The polypeptide of claim 6, wherein the polypeptide comprises an amino acid sequence having at least 85% identity to the amino acid sequence SEQ ID NO: 17 or SEQ ID NO: 18.


SEQUENCE LISTING (30 sequences)

SEQ ID NO: 1 - protein, 736 aa, Homo sapiens
MEIGTEISRKIRSAIKGKLQELGAYVDEELPDYIMVMVANKKSQDQMTEDLSLFLGNNTIRFTVWLHGVLDKLRSVTTEPSSLKSSDTNIFDSNVPSNKSNFSRGDERRHEAAVPPLAIPSARPEKRDSRVSTSSQESKTTNVRQTYDDGAATRLMSTVKPLREPAPSEDVIDIKPEPDDLIDEDLNFVQENPLSQKKPTVTLTYGSSRPSIEIYRPPASRNADSGVHLNRLQFQQQQNSIHAAKQLDMQSSWVYETGRLCEPEVLNSLEETYSPFFRNNSEKMSMEDENFRKRKLPVVSSVVKVKKFNHDGEEEEEDDDYGSRTGSISSSVSVPAKPERRPSLPPSKQANKNLILKAISEAQESVTKTTNYSTVPQKQTLPVAPRTRTSQEELLAEVVQGQSRTPRISPPIKEEETKGDSVEKNQGTQQRQLLSRLQIDPVMAETLQMSQDYYDMESMVHADTRSFILKKPKLSEEVVVAPNQESGMKTADSLRVLSGHLMQTRDLVQPDKPASPKFIVTLDGVPSPPGYMSDQEEDMCFEGMKPVNQTAASNKGLRGLLHPQQLHLLSRQLEDPNGSFSNAEMSELSVAQKPEKLLERCKYWPACKNGDECAYHHPISPCKAFPNCKFAEKCLFVHPNCKYDAKCTKPDCPFTHVSRRIPVLSPKPAVAPPAPPSSSQLCRYFPACKKMECPFYHPKHCRFNTQCTRPDCTFYHPTINVPPRHALKWIRPQTSE

SEQ ID NO: 2 - protein, 735 aa, Mus musculus
MEIGTEISRKIRSAIKGKLQELGAYVDEELPDYIMVMVANKKSQDQMTEDLSLFLGNNTIRFTVWLHGVLDKLRSVTTEPSSLKSPDASIFDSHVPSNKSSFSRGDERRHEAAVPPLAVSSSRPEKRDSRVSTSSQEQKSTNVRHSYDDGASTRLMSTVKPLREPAPSEDVIDIKPEPDDLIDEDLNFVQENPLSQKKPTVTLTYGSSRPSIEIYRPPASRNADTGTHLNRLQLHPQQSSAHAAKQLDVQSSQVSEAGRLCEPPVLSSVEDTYSPFFRNNLDKMSIEDENFRKRKLPVVSSVVKVKRFSHDGEEEEEDEDYGTRIGSLSSSVSVPAKPERRPSLPPSKQANKNLILKAISEAQESVTKTTNYSAVPQKQTLPVAPRTRTSQEELLAEMVQGQNRAPRISPPVKEEEAKGDNTGKSQGTQQRQLLSRLQIDPVMVETMEMSQDYYDMESMVHADTRSFILKKPKLSEEIVVTPNQDSGMKTADALRVLSGHLMQTRDLVQPDKPASPKFIVTLDGVPSPPGYMSDQEEEMCFEGMKPVNQTSASNKGLRGLLHPQQLHLLSRQLEDPDGSFSNAEMTDLSVAQKPEKLLERCKYWPACKNGDECVYHHPISPCKAFPNCKFAEKCLFVHPNCKYDTKCTKADCPFTHMSRRASILTPKPVSSPAPSSNGQLCRYFPACKKMECPFYHPKHCRFNTQCTRPDCTFYHPTITVPPRHALKWIRPQSSE

SEQ ID NO: 3 - protein, 736 aa, Rattus norvegicus
MEIGTEISRKIRSAIKGKLQELGAYVDEELPDYIMVMVANKKSQDQMTEDLSLFLGNNTIRFTVWLHGVLDKLRSVTTEPSSLKSPDTSIFDSNVPSNKSSFSRGDERRHEAAIPPLAVSSSRPEKRDSRVSTSSQEHKSTNVRHSYDDGASTRLMSTVKPLREPAPSEDVIDIKPEPDDLIDEDLNFVQENSLSQKKPTVTLTYGSSRPSIEIYRPPASRNADTGTHLNRPQLQQQQSSTHTAKQLDGQSSQVYEAGRLCEPEVLGSVEDTYSPFFRNNLDKMNIEEENFRKRKLPVVSSVVKVKRFSHDGEEEEEDEDYGTRVGSLSSSVSVPAKPERRPSLPPSKQANKNLILKAISEAQESVTKTTNYPAVPQKQTLPVAPRTRTSQEEVLAEMVQGQNRAPRISPPVKEEEAKGDNAEKIEGTQQRQLLSRLQIDPVTVDTMELSQDYYDMESMVHADTRSFILKKPKLSEEIVVTPNQDSGMKTADALRVLSGHLMQTRDLVQPDKPASPKFIVTLDGVPSPPGYMSDQEEEMCFEGMKPVNQTSASNKGLRGLLHPQQLHLLSRQLEDPDGSFSNAEMTDLSVAQKPEKLLERCKYWPACKNGDECVYHHPISPCKAFPNCKFAEKCLFVHPNCKYDAKCTKADCPFTHMSRRGPVLTPKPAVSSPAPSSNGQFCRYFPACKKMECPFYHPKHCRFNTQCTRPDCTFYHPTITVPPRHALKWIRPQTSE

SEQ ID NO: 4 - protein, 730 aa, Gallus gallus
MEIGTEISRKIRGAIKGKLQELGAYVDEELPDYIMVMVANKKSQEQMTEDLSLFLGNNTVRFTVWLHGVLDKLRSVTTEPSGTKSSEPNIFESNHSSSKSSSCVSDERRREDTLPPLAVSSTRSERTDSRVSTSSQEQRNTASRQSCEDGSASRLMSTVKPLRELSPSEAVIDIKPEPDDLIDEDLNFVQENPLSRKKPIVTVTYGSSRPTAEIYRPPASRSADGSLQVHRLPQQGNLQGNRQLDTQSCRSLETVQLCNPEAFGSLAESYRPTSKLSADKVGSEEEGSRKRRLPIVSSVVKVKKFCNDGEEEEEEDDYGLRTGSISSSVSVPAKPERRPSLPPSKQVNKNLILKAISEAQESVTKTTNYSAVPQKQTVPVAPRTRISPEESHLEVIHLQSRLPALCSQLQVEEPKEQAVEGIQGAEQKELSSRLQVDPVIEDTLQVTQDYYDGESMVHTDTRSFILKKPKLSEEIAAQNQQLGKRATEAMRVLSGRLIQTRDQIAQPEKPASPKFIVTLDGVPSPPGYLSDQEEEDMYITEGLKPIPQNICAGKGLKGLRAQQMQIVTRQLDSSDVEMEQLNVLQKQEKVLERCKYWPACKNGDECVYHHPTQPCKVFPNCKFADKCLFIHPNCKYDAKCTKPDCPYTHASRRNPLPSPKPVPLPTQSVSSSSPLCKFFPACKKMECPFYHPKHCRFNTQCTRPDCTFYHPTIAVPPRHALKWTRTQTSE

SEQ ID NO: 5 - protein, 736 aa, Pan troglodytes
MEIGTEISRKIRSAIKGKLQELGAYVDEELPDYIMVMVANKKSQDQMTEDLSLFLGNNTIRFTVWLHGVLDKLRSVTTEPSSLKSSDTNIFDSNVPSNKSNFSRGDERRHEAAVPPLAIPSARPEKRDSRVSTSSQESKTTNVRQTYDDGAATRLMSTVKPLREPAPSEDVIDIKPEPDDLIDEDLNFVQENPLSQKKPTVTLTYGSSRPSIEIYRPPASRNADSGVHLNRLQFQQQQNSIHAAKQLDMQSSWVYETGRLCEPEVLNSLEETYSPFFRNNSEKMSMEDENFRKRKLPVVSSVVKVKKFNHDGEEEEEDDDYGSRTGSISSSVSVPAKPERRPSLPPSKQANKNLILKAISEAQESVTKTTNYSTVPQKQTLPVAPRTRTSQEELLAEVVQGQSRTPRISPPIKEEETKGDSVEKNQGTQQRQLLSRLQIDPVMAETLQMSQDYYDMESMVHADTRSFILKKPKLSEEIVVAPNQESGMKTADSLRVLSGHLMQTRDLVQPDKPASPKFIVTLDGVPSPPGYMSDQEEDMCFEGMKPVNQTAASNKGLRGLLHPQQLHLLSRQLEDPNGSFSNAEMSELSVAQKPEKLLERCKYWPACKNGDECAYHHPISPCKAFPNCKFAEKCLFVHPNCKYDAKCTKPDCPFTHVSRRIPVLSPKPAVAPPAPPSSSQLCRYFPACKKMECPFYHPKHCRFNTQCTRPDCTFYHPTINVPPRHALKWIRPQTSE

SEQ ID NO: 6 - protein, 736 aa, Canis lupus
  source 1..736: note = Canis lupus familiaris
MEIGTEISRKIRSAIKGKLQELGAYVDEELPDYIMVMVANKKSQDQMTEDLSLFLGNNTIRFTVWLHGVLDKLRSVTTDPSSLKSSDTNIFDNNVSSNKSSFSRGDERRHEAAVPPLAVASTRPEKRDSRVSTSSQEQKTTNVRQTYDDGAATRLMSTVKPLREPAPSEDVIDIKPEPDDLIDEDLNFVQENPLSQKKPTVTLTYGSSRPSIEIYRPPASRNADSGAHLNRLQFQQQQNSIHAAKQPDIQNSRVYETGRLCEPEVLNSLEDTYSPFFRNNSEKMSIEEENFRKRKLPVVSSVVKVKKFNHDGEEEEEDDDCGSRTGSISSSVSVPAKPERRPSLPPSKQANKNLILKAISEAQESVTKTTNYSTVSQKQTLPVAPRTRTSQEELLAEMVQGQSRTPRISPPMKEEETKGDNIEKSQGTQQRQLLSRLQIDPVMAETLQISQDYYDMESMVHADTRSFILKKPKLSEEIVVASNQESGMKTADTLRVLSGHLMQTRDLVQPDKPASPKFIVTLDGVPSPPGYMSDQEEDMCFEGVKPVNQTAASNKGLRGLLHPQQLQLMSRQLDDPNGSFLNAEMSELSVVQKPEKLLERCKYWPACKNGDECAYHHPVSPCKAFPNCKFAEKCLFVHPNCKYDAKCTKPDCPFTHMSRRIPVLPPKPAIATPAPPSSSQLCRYFPACKKMECPFYHPKHCRFNTQCTRPDCTFYHPTITVPPRHALKWIRPQTSE

SEQ ID NO: 7 - protein, 735 aa, Bos taurus
MEIGTEISRKIRSAIKGKLQELGAYVDEELPDYIMVMVANKKSQDQMTEDLSLFLGNNTIRFTVWLHGVLDKLRSVTTDPASLKSSDTNLFDGNVPSNKSSFSRGDERRHEAAVPPLAVSSTRPEKRESRVSTSSQEQKATNVRQTYDDGAATRLMSTVKPLRELAPSEDVIDIKPEPDDLIDEDLNFVQENPLSQKKTTVTLTYGSSRPSIEIYRPPATRNTDSGAHLNRLQFQQQQNSIHAAKQLDIQSSRVYETGRLCEPEVLNSLEETYSPFFRSNAEKMSIEEENFRKRKLPVVSSVVKVKKFSHDGEEEEEDDDCGSRTGSISSSVSVPAKPERRPSLPPSKQANKNLILKAISEAQESVTKTTNYSTVSQKQTLPVAPRTRTSQEDLLAEVAQGHGRVPRISSPVKEEEAQGGSVDERQGTQQRQLLSRLQIDPVMAETLQISQDYYDMESMVHADTRSFILKKPKLCEELVVAASQASGMETADALRARSGHLVQTRDLVQPDKPASPKFIVTLDGVPSPPGYMSDQEEDMCSEGMRPAQHPAASHGGLAGLLHPQRSRVLSRQLEDPDGSFANAEMSELSVAQKPEKLLERCKYWPACKNGDECAYHHPVSPCKAFPNCKFAEKCLFVHPNCKYDAKCTKPDCPFTHMSRRTPGLPPKPVTAPAPPSSSQLCRYFPACKKMECPFYHPKHCRFNTQCTRPDCAFYHPTITVPPRHALKWIRPQTSD

SEQ ID NO: 8 - protein, 669 aa, Danio rerio
MEIGTEISKKIRTAIKRKLQEFGAYVDEELPDYIMVMVANKKNPQQMADDLSLFLGNNTIKFTVWLHGVLEKLRSVAVEPPSLGPSVVHSETSIPAENSRRGAEPRAIAVSSSRSDKAEGRVSSSAHENRASKRGSSERPSRLTSAVKPLMEASAEAVIDIKPDLDDDLISYDPVEHSLTSGHSQALYSRSTAERQRPAVESSRQTADTYRSSDISRGQDRSERGYRSSAESSRDLSRKRKAPVASSVVRVHRGHERGLEIEDLEEEEEDEDYGLASKVSLPSKPERKPTLPPAKQANKNLILKAISEAQESINKTTSQYTVPQRQTVPVAPRTRSASDEMSNAAIRLVQEHLHALTPQDTLHNTQSRGLASRLQLEVPEEDSREPHEYELQVLEAARLKALDTRSFIMRQPEVEQPPPIRSRLSAVNQNENAPTTSRMVQARERAEAVGGSSPKFIVTLDGVPSPLANRTDQEMETEDELNTTADLPENNNNNNTTTTTSKPAIHLRLGADFRNACDDEVEEMDVEAVQAKRQKLPERCKFWPTCKSGDECLYHHPNTQCKVFPNCKFADKCLFIHPNCKFDAKCTKADSPFTHVSRRLNSNPTRAAPALTSTVCRFFPGCKKVDCPFYHPKPCRFATQCKRADCTFYHPAVAVPPRSALKWTKTQSS

SEQ ID NO: 9 - protein, 736 aa, Macaca mulatta
MEIGTEISRKIRSAIKGKLQELGAYVDEELPDYIMVMVANKKSQDQMTEDLSLFLGNNTIRFTVWLHGVLDKLRSVTTEPSSLKSSDTNIFDSNVPSNKSNFSRGDERRHEAAVPPLAIPSTRPEKRDSRVSTSSQESKTTNVRQTYDDGAATRLMSTVKPLREPAPSEDVIDIKPEPDDLIDEDLNFVQENPLSQKKPTVTLTYGSSRPSIEIYRPPASRNADSGVHLNRLQFQQQQNSIHAAKQLDMQNSWVYETGRLCEPEVLNSLEETYSPFFRNNSEKMSMEDENFRKRKLPVVSSVVKVKKFNHDGEEEEEDDDYGSRTGSISSSVSVPAKPERRPSLPPSKQANKNLILKAISEAQESVTKTTNYSTVPQKQTLPVAPRTRTSQEELLAEVVQGQSRTPRISPPIKEEETKGDSVEKNQGTQQRQLLSRLQIDPVMAETLQMSQDYYDMESMVHADTRSFILKKPKLSEEIVVAPNQESGMKTADSLRVLSGHLMQTRDLVQPDKPASPKFIVTLDGVPSPPGYMSDQEEDMCFEGMKPVNQTAASNKGLRGLLHPQQLHLLSRQLEDPNGSFSNAEMSELSVAQKPEKLLERCKYWPACKNGDECAYHHPISPCKAFPNCKFAEKCLFVHPNCKYDAKCTKPDCPFTHVSRRIPVLSPKPAVAPPAPPSSSQLCRYFPACKKMECPFYHPKHCRFNTQCTRPDCTFYHPTINVPPRHALKWIRPQTSE

SEQ ID NO: 10 - protein, 736 aa, Equus caballus
MEIGTEISRKIRSAIKGKLQELGAYVDEELPDYIMVMVANKKSQDQMTEDLSLFLGNNTIRFTVWLHGVLDKLRSVTTDPSSLKSSDTNIFDNNVPSNKSNFSRGDERRHEAAVPPLAVSSTRPEKRDSRVSTSSQEQKTTSVRQTYDDGAATRLMSTVKPLREPAPSEDVIDIKPEPDDLIDEDLNFVQENPLSQKKPTVTLTYGSSRPSIEIYRPPASRNADSGAHLNRLQFQQQQNSIRAVKQLDMQNSRVYETGRLCEPEVLNSLEETYSPFFRNNAEKMSIEEENFRKRKLPVVSSVVKVKKFNHDGEEEEEDDDCGSRTGSISSSVSVPAKPERRPSLPPSKQANKNLILKAISEAQESVTKTTNYSTVSQKQTLPVAPRTRTSQEELLAEMVQGQSKTPRISPPVKEEEIKGDNIEKSQGTQQRQLLSRLQIDPVVAETLQISQDYYDMESMVHADTRSFILKKPKLSEEIVVAPNQESGMKTADTLRVLSGHLMQTRDLVQPDKPASPKFIVTLDGVPSPPGYMSDQEEDMCFEGMKPVNQTAASNKGLRGLLHPQQLHLMSRQLDDPNGSFSNAEMSELSVAQKPEKLLERCKYWPACKNGDECAYHHPVSPCKAFPNCKFAEKCLFVHPNCKYDAKCTKPDCPFTHMSRRIPVLPPKPAVTTPASPSSSQLCRYFPACKKMECPFYHPKHCRFNTQCTRPDCTFYHPTITVPPRHALKWIRPQTSE

SEQ ID NO: 11 - protein, 735 aa, Sus scrofa
MEIGTEISRKIRSAIKGKLQELGAYVDEELPDYIMVMVANKKSQDQMTEDLSLFLGNNTIRFTVWLHGVLDKLRSVTTDPSSFKSSDTNIFDSNVPSNKSSFSRGDERRHEAAVPPLAVSSTRPEKRDSRVSTSSQEQKATNVRQTYDDGAATRLMSTVKPLREPAPSEDVIDIKPEPDDLIDEDLNFVQENPLSQKKPTVTLTYGSSRPSIEIYRPPASRNADSGAHLNRLQFQQQQNSIHAAKQVDIQSSRVYETGRLCEPEVLNSLEETYSPFFRNNSEKMSIEEENFRKRKLPVVSSVVKVKKFNHDGEEEEEEDDCGSRTGSISSSVSVPAKPERRPSLPPSKQANKNLILKAISEAQESVTKTTNYSTVSQKQTLPVAPRTRTSQEELLAEMVQGQSKAPRISSPIKEEETKGDNIDKSQGTQQRQLLSRLQIDPVMAETLQISQDYYDMESMVHADTRSFILKKPKLSEEIVVAPNQESGMKTADTLRVLSGHLMQTRDLVQPDKPASPKFIVTLDGVPSPPGYMSDQEEDMCFEGMRPAHHTAASHEGLAGLLHPQQLHLLSRQLEDPDGSFSNAEMSELSVAQKPEKLLERCKYWPACKNGDECAYHHPISPCKAFPNCKFAEKCLFVHPNCKYDAKCTKPDCPFTHMSRRIPVLPPKPVTTPAPPSSSQLCRYFPACKKMECPFYHPKHCRFNTQCTRPDCTFYHPTITVPPRHALKWIRPQTSE

SEQ ID NO: 12 - protein, 735 aa, Oryctolagus cuniculus
MEIGTEISRKIRSAIKGKLQELGAYVDEELPDYIMVMVANKKSQDQMTEDLSLFLGNNTIRFTVWLHGVLDKLRSVTTEPSSLKSSDTNIFDSNVPSTKSSFSRGDERRHEATVPPLAVSSSRPEKRDSRVSTSSQEQKTTVRQTYDDGAATRLMSTVKPLREPAPSEDVIDIKPEPDDLIDEDLNFVQENPLSQKKPTVTLTYGSSRPSIEIYRPPASRSSDGGAHLNRLQFQQQQNSGHAGKQLDAQNSRVYETGRLCEPEVLSSLEETYSPFFKNSSEKMGIEDENFRKRKLPVVSSVVKVKKFNLDGEEEEEDDDYGSRTGSVSSSVSVPAKPERRPSLPPSKQANKNLILKAISEAQESVTKTTNYSAVPQKQTLPVAPRTRTSQEELLPEVVQGQSRIPRISPPMKEEETKGDNIEKSQGTQQRQLLSRLQIDPAMAETLQISQDYYDMESMVHADTRSFILKKPKLSEEIVVAPNQESGMKTADTLRVLSGHLMQTRDLVQPDKPASPKFIVTLDGVPSPPGYMSDQEEDMCFEGVKPVNQTAASHQGLRGLLHPQQLQLMSRQLDDPDGSFANAEMSELSVAQKPEKLLERCKYWPACKNGDECAYHHPVSPCKAFPNCKFAEKCLFVHPNCKYDAKCTKPDCPFTHVNRRIPALPPKPAVTTPGIPSSSQLCRYFPACKKMECPFYHPKHCRFNTQCTRPDCTFYHPTITVPPRHALKWIRPQTSE

SEQ ID NO: 13 - protein, 735 aa, Cavia porcellus
MEIGTEISRKIRSAIKGKLQELGAYVDEELPDYIMVMVANKKSQDQMTEDLSLFLGNNTIRFTVWLHGVLDKLRSVTTEPSSLKSSDTNIFDNNVPSNKSSFSRGDERRHEATVPPLAISSTRPEKRDSRVAASSQEQKTSNLRQTYDDGAATRLMSTVKPLREPAPSEDVIDIKPEPDDLIDEDLNFVPENPLSQKKPTVTLTYGSSRPSIEIYRPPASRNAESSAHLNRLQFQQQQNSIHAAKQLDMQNSRVYETEHTCEPEVLNSLEETYSPFFRNNSEKMMIEEENFRKRKLPVVSSVVKVKKFNHDGEEEEEDDDYGSRTGSVSSSVSVPAKPERRPSLPPSKQANKNLILKAISEAQESVTKTTNYSAVPQKQTLPVAPRTRTSQEELLAEVVQGQSRTSRVSPPIKEEETKGDNAEKNQGAQQRQLLSRLQIDPVMAETLQSQDYYDMESMVHADTRSFILKKPKLSEEIVVAPNQESGMKTADTLRVLSGHLMQTRDLVQPDKPASPKFIVTLDGVPSPPGYMSDQEEDMCFEGVKPVNQTAASNKGLRGLLHPQQLQLMQRQLGDPDGSFSYVEMSELNVAQKPEKLLERCKYWPACKNGDECAYHHPVSPCKAFPNCKFAEKCLFVHPNCKYDAKCTKPDCPFTHINRRIPVLPPKPAVTTTASPSTSQLCRYFPACKKMECPFYHPKHCRFNTQCTRPDCTFYHPTITVPPRHALKWIRPQTSE

SEQ ID NO: 14 - protein, 735 aa, Cricetulus barabensis
  source 1..735: note = Cricetulus griseus
MEIGTEISRKIRSAIKGKLQELGAYVDEELPDYIMVMVANKKSQDQMTEDLSLFLGNNTIRFTVWLHGVLDKLRSVTTEPSSLKSSDTSIFDSNVPSNKSSFSRGDERRHEAAVPPLAVASSRPEKRESRVSATSQEQKSTHVRHSYDDGGSTRLMSTVKPLREPAPSEDVIDIKPEPDDIDEDLNFVQENPLSQKKPTVTLTYGSSRPSIEIYRPPASRNADTGTHLNRLQLHQQQNSTHTAKQLDVQSSQVYEAGRLCEPEVLSSVDDTYSPFFRNNLDKMSIEDENFRKRKLPVVSSVVKVKRFSHDGEEEEEDEDYGSRIGSLSSSVSVPAKPERRPSLPPSKQANKNLILKAISEAQESVTKTTNYSAVPQKQTLPVAPRTRTSQEELIAEMVQGQSRAPRISPPVKEEEAKGDSTEKIQGTQQRQLLSRLQIDPVMVETMEMSPDYYDMESMVHADTRSFILKKPKLSEEIVVTPNQESGMKTADALRVLSGHLMQTRDLVQPDKPASPKFIVTLDGVPSPPGYMSDQEEEMCFEGVKPVNQTAASNKGLRGLLHPQHLHLMSRQLEDPDGSFSNAEMTDLSVAQKPEKLLERCKYWPACKNGDECAYHHPISPCKAFPNCKFAEKCLFVHPNCKYDAKCTKPDCPFTHMSRRIPVLASKPVVSSPAPSSNGQLCRYFPACKKMECPFYHPKHCRFNTQCTRPDCTFYHPTITVPPRHALKWIRPQTSE

SEQ ID NO: 15 - protein, 736 aa, Felis catus
MEIGTEISRKIRSAIKGKLQELGAYVDEELPDYIMVMVANKKSQDQMTEDLSLFLGNNTIRFTVWLHGVLDKLRSVTTDPSSLKSSDTNIFDNNVSSNKSSFGRGDERRHEAAVPPLAVASTRPEKRDSRVSTSSQEQKTTNVRQTYDDGAATRLMSTVKPLREPAPSEDVIDIKPEPDDLIDEDLNFVQENPLSQKKPTVTLTYGSSRPSIEIYRPPASRNADSGAHLNRLQFQQQQNNIHAAKQPDLQNSRVYETGRLCEQEVLNSLEETYSPFFRNNSEKMSIEEENFRKRKLPVVSSVVKVKKFNHDGEEEEEDDDCGSRTGSISSSVSVPAKPERRPSLPPSKQANKNLILKAISEAQESVTKTTNYSTVSQKQTLPVAPRTRTSQEELLAEMVQGQSRTPRISPPIKEEETKGDNIEKSQGAPQRQLLSRLQIDPVMAETLQISQDYYDMESMVHADTRSFILKKPKLSEEIVVAPNQESGMKTADTLRVLSGHLMQTRDLVQPDKPASPKFIVTLDGVPSPPGYMSDQEEDMCFEGMKPVNQTAAASKGLRGLLHPQQLQLMSRQLDDPNGSFANAEMSELSVAQKPEKLLERCKYWPACKNGDECAYHHPVSPCKAFPNCKFAEKCLFVHPNCKYDAKCTKPDCPFTHMSRRIPVLPLKPAVTTPAPPSSSQLCRYFPACKKMECPFYHPKHCRFNTQCTRPDCTFYHPTITVPPRHALKWIRPQTSE

SEQ ID NO: 16 - protein, 736 aa, synthetic construct
MEIGTEISRKIRSAIKGKLQELGAYVDEELPDYIMVMVANKKSQDQMTEDLSLFLGNNTIRFTVWLHGVLDKLRSVTTEPSSLKSSDTNIFDSNVPSNKSNFSRGDERRHEAAVPPLAIPSARPEKRDSRVSTSSQESKTTNVRQTYDDGAATRLMSTVKPLREPAPSEDVIDIKPEPDDLIDEDLNFVQENPLSQKKPTVTLTYGSSRPSIEIYRPPASRNADSGVHLNRLQFQQQQNSIHAAKQLDMQSSWVYETGRLCEPEVLNSLEETYSPFFRNNSEKMSMEDENFRKRKLPVVSSVVKVKKFNHDGEEEEEDDDYGSRTGSISSSVSVPAKPERRPSLPPSKQANKNLILKAISEAQESVTKTTNYSTVPQKQTLPVAPRTRTSQEELLAEVVQGQSRTPRISPPIKEEETKGDSVEKNQGTQQRQLLSRLQIDPVMAETLQMSQDYYDMESMVHADTRSFILKKPKLDEEVVVAPNQESGMKTADSLRVLSGHLMQTRDLVQPDKPASPKFIVTLDGVPSPPGYMSDQEEDMCFEGMKPVNQTAASNKGLRGLLHPQQLHLLSRQLEDPNGSFSNAEMSELSVAQKPEKLLERCKYWPACKNGDECAYHHPISPCKAFPNCKFAEKCLFVHPNCKYDAKCTKPDCPFTHVSRRIPVLSPKPAVAPPAPPSSSQLCRYFPACKKMECPFYHPKHCRFNTQCTRPDCTFYHPTINVPPRHALKWIRPQTSE

SEQ ID NO: 17 - protein, 736 aa, synthetic construct
MEIGTEISRKIRSAIKGKLQELGAYVDEELPDYIMVMVANKKSQDQMTEDLSLFLGNNTIRFTVWLHGVLDKLRSVTTEPSSLKSSDTNIFDSNVPSNKSNFSRGDERRHEAAVPPLAIPSARPEKRDSRVSTSSQESKTTNVRQTYDDGAATRLMSTVKPLREPAPSEDVIDIKPEPDDLIDEDLNFVQENPLSQKKPTVTLTYGSSRPSIEIYRPPASRNADSGVHLNRLQFQQQQNSIHAAKQLDMQSSWVYETGRLCEPEVLNSLEETYSPFFRNNSEKMSMEDENFRKRKLPVVSSVVKVKKFNHDGEEEEEDDDYGSRTGSISSSVSVPAKPERRPSLPPSKQANKNLILKAISEAQESVTKTTNYSTVPQKQTLPVAPRTRTSQEELLAEVVQGQSRTPRISPPIKEEETKGDSVEKNQGTQQRQLLSRLQIDPVMAETLQMSQDYYDMESMVHADTRSFILKKPKLEEEVVVAPNQESGMKTADSLRVLSGHLMQTRDLVQPDKPASPKFIVTLDGVPSPPGYMSDQEEDMCFEGMKPVNQTAASNKGLRGLLHPQQLHLLSRQLEDPNGSFSNAEMSELSVAQKPEKLLERCKYWPACKNGDECAYHHPISPCKAFPNCKFAEKCLFVHPNCKYDAKCTKPDCPFTHVSRRIPVLSPKPAVAPPAPPSSSQLCRYFPACKKMECPFYHPKHCRFNTQCTRPDCTFYHPTINVPPRHALKWIRPQTSE

SEQ ID NO: 18 - protein, 736 aa, synthetic construct
MEIGTEISRKIRSAIKGKLQELGAYVDEELPDYIMVMVANKKSQDQMTEDLSLFLGNNTIRFTVWLHGVLDKLRSVTTEPSSLKSSDTNIFDSNVPSNKSNFSRGDERRHEAAVPPLAIPSARPEKRDSRVSTSSQESKTTNVRQTYDDGAATRLMSTVKPLREPAPSEDVIDIKPEPDDLIDEDLNFVQENPLSQKKPTVTLTYGSSRPSIEIYRPPASRNADSGVHLNRLQFQQQQNSIHAAKQLDMQSSWVYETGRLCEPEVLNSLEETYSPFFRNNSEKMSMEDENFRKRKLPVVSSVVKVKKFNHDGEEEEEDDDYGSRTGSISSSVSVPAKPERRPSLPPSKQANKNLILKAISEAQESVTKTTNYSTVPQKQTLPVAPRTRTSQEELLAEVVQGQSRTPRISPPIKEEETKGDSVEKNQGTQQRQLLSRLQIDPVMAETLQMSQDYYDMESMVHADTRSFILKKPKLAEEVVVAPNQESGMKTADSLRVLSGHLMQTRDLVQPDKPASPKFIVTLDGVPSPPGYMSDQEEDMCFEGMKPVNQTAASNKGLRGLLHPQQLHLLSRQLEDPNGSFSNAEMSELSVAQKPEKLLERCKYWPACKNGDECAYHHPISPCKAFPNCKFAEKCLFVHPNCKYDAKCTKPDCPFTHVSRRIPVLSPKPAVAPPAPPSSSQLCRYFPACKKMECPFYHPKHCRFNTQCTRPDCTFYHPTINVPPRHALKWIRPQTSE

SEQ ID NO: 19 - DNA, 2898 bp, synthetic construct
  source 1..2898: mol_type = other DNA
atcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattcc

SEQ ID NO: 20 - protein, 857 aa, Homo sapiens
MEAGPSGAAAGAYLPPLQQVFQAPRRPGIGTVGKPIKLLANYFEVDIPKIDVYHYEVDIKPDKCPRRVNREVVEYMVQHFKPQIFGDRKPVYDGKKNIYTVTALPIGNERVDFEVTIPGEGKDRIFKVSIKWLAIVSWRMLHEALVSGQIPVPLESVQALDVAMRHLASMRYTPVGRSFFSPPEGYYHPLGGGREVWFGFHQSVRPAMWKMMLNIDVSATAFYKAQPVIEFMCEVLDIRNIDEQPKPLTDSQRVRFTKEIKGLKVEVTHCGQMKRKYRVCNVTRRPASHQTFPLQLESGQTVECTVAQYFKQKYNLQLKYPHLPCLQVGQEQKHTYLPLEVCNIVAGQRCIKKLTDNQTSTMIKATARSAPDRQEEISRLMKNASYNLDPYIQEFGIKVKDDMTEVTGRVLPAPILQYGGRNRAIATPNQGVWDMRGKQFYNGIEIKVWAIACFAPQKQCREEVLKNFTDQLRKISKDAGMPIQGQPCFCKYAQGADSVEPMFRHLKNTYSGLQLIIVILPGKTPVYAEVKRVGDTLLGMATQCVQVKNVVKTSPQTLSNLCLKINVKLGGINNILVPHQRSAVFQQPVIFLGADVTHPPAGDGKKPSITAVVGSMDAHPSRYCATVRVQRPRQEIIEDLSYMVRELLIQFYKSTRFKPTRIIFYRDGVPEGQLPQILHYELLAIRDACIKLEKDYQPGITYIVVQKRHHTRLFCADKNERIGKSGNIPAGTTVDTNITHPFEFDFYLCSHAGIQGTSRPSHYYVLWDDNRFTADELQILTYQLCHTYVRCTRSVSIPAPAYYARLVAFRARYHLVDKEHDSGEGSHISGQSNGRDPQALAKAVQVHQDTLRTMYFA

SEQ ID NO: 21 - protein, 859 aa, Homo sapiens
MYSGAGPALAPPAPPPPIQGYAFKPPPRPDFGTSGRTIKLQANFFEMDIPKIDIYHYELDIKPEKCPRRVNREIVEHMVQHFKTQIFGDRKPVFDGRKNLYTAMPLPIGRDKVELEVTLPGEGKDRIFKVSIKWVSCVSLQALHDALSGRLPSVPFETIQALDVVMRHLPSMRYTPVGRSFFTASEGCSNPLGGGREVWFGFHQSVRPSLWKMMLNIDVSATAFYKAQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQRCIKKLTDNQTSTMIRATARSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFA

SEQ ID NO: 22 - protein, 860 aa, Homo sapiens
MEIGSAGPAGAQPLLMVPRRPGYGTMGKPIKLLANCFQVEIPKIDVYLYEVDIKPDKCPRRVNREVVDSMVQHFKVTIFGDRRPVYDGKRSLYTANPLPVATTGVDLDVTLPGEGGKDRPFKVSIKFVSRVSWHLLHEVLTGRTLPEPLELDKPISTNPVHAVDVVLRHLPSMKYTPVGRSFFSAPEGYDHPLGGGREVWFGFHQSVRPAMWKMMLNIDVSATAFYKAQPVIQFMCEVLDIHNIDEQPRPLTDSHRVKFTKEIKGLKVEVTHCGTMRRKYRVCNVTRRPASHQTFPLQLENGQTVERTVAQYFREKYTLQLKYPHLPCLQVGQEQKHTYLPLEVCNIVAGQRCIKKLTDNQTSTMIKATARSAPDRQEEISRLVRSANYETDPFVQEFQFKVRDEMAHVTGRVLPAPMLQYGGRNRTVATPSHGVWDMRGKQFHTGVEIKMWAIACFATQRQCREEILKGFTDQLRKISKDAGMPIQGQPCFCKYAQGADSVEPMFRHLKNTYSGLQLIIVILPGKTPVYAEVKRVGDTLLGMATQCVQVKNVIKTSPQTLSNLCLKINVKLGGINNILVPHQRPSVFQQPVIFLGADVTHPPAGDGKKPSIAAVVGSMDAHPSRYCATVRVQRPRQEIIQDLASMVRELLIQFYKSTRFKPTRIIFYRDGVSEGQFRQVLYYELLAIREACISLEKDYQPGITYIVVQKRHHTRLFCADRTERVGRSGNIPAGTTVDTDITHPYEFDFYLCSHAGIQGTSRPSHYHVLWDDNCFTADELQLLTYQLCHTYVRCTRSVSIPAPAYYAHLVAFRARYHLVDKEHDSAEGSHVSGQSNGRDPQALAKAVQIHQDTLRTMYFA

SEQ ID NO: 23 - protein, 861 aa, Homo sapiens
MEALGPGPPASLFQPPRRPGLGTVGKPIRLLANHFQVQIPKIDVYHYDVDIKPEKRPRRVNREVVDTMVRHFKMQIFGDRQPGYDGKRNMYTAHPLPIGRDRVDMEVTLPGEGKDQTFKVSVQWVSVVSLQLLLEALAGHLNEVPDDSVQALDVITRHLPSMRYTPVGRSFFSPPEGYYHPLGGGREVWFGFHQSVRPAMWNMMLNIDVSATAFYRAQPIIEFMCEVLDIQNINEQTKPLTDSQRVKFTKEIRGLKVEVTHCGQMKRKYRVCNVTRRPASHQTFPLQLENGQAMECTVAQYFKQKYSLQLKYPHLPCLQVGQEQKHTYLPLEVCNIVAGQRCIKKLTDNQTSTMIKATARSAPDRQEEISRLVKSNSMVGGPDPYLKEFGIVVHNEMTELTGRVLPAPMLQYGGRNKTVATPNQGVWDMRGKQFYAGIEIKVWAVACFAPQKQCREDLLKSFTDQLRKISKDAGMPIQGQPCFCKYAQGADSVEPMFKHLKMTYVGLQLIVVILPGKTPVYAEVKRVGDTLLGMATQCVQVKNVVKTSPQTLSNLCLKINAKLGGINNVLVPHQRPSVFQQPVIFLGADVTHPPAGDGKKPSIAAVVGSMDGHPSRYCATVRVQTSRQEISQELLYSQEVIQDLTNMVRELLIQFYKSTRFKPTRIIYYRGGVSEGQMKQVAWPELIAIRKACISLEEDYRPGITYIVVQKRHHTRLFCADKTERVGKSGNVPAGTTVDSTITHPSEFDFYLCSHAGIQGTSRPSHYQVLWDDNCFTADELQLLTYQLCHTYVRCTRSVSIPAPAYYARLVAFRARYHLVDKDHDSAEGSHVSGQSNGRDPQALAKAVQIHHDTQHTMYFA

SEQ ID NO: 24 - protein, 70 aa, synthetic construct
  REGION 1..70: note = Description of Artificial Sequence: Synthetic polypeptide
  VARIANT 1..70: note = This sequence may encompass 1-10 repeating (G)nS units, wherein n = 2-6
  VARIANT 1..6: note = This region may encompass 2-6 residues
  VARIANT 8..13: note = This region may encompass 2-6 residues
  VARIANT 15..20: note = This region may encompass 2-6 residues
  VARIANT 22..27: note = This region may encompass 2-6 residues
  VARIANT 29..34: note = This region may encompass 2-6 residues
  VARIANT 36..41: note = This region may encompass 2-6 residues
  VARIANT 43..48: note = This region may encompass 2-6 residues
  VARIANT 50..55: note = This region may encompass 2-6 residues
  VARIANT 57..62: note = This region may encompass 2-6 residues
  VARIANT 64..69: note = This region may encompass 2-6 residues
GGGGGGSGGGGGGSGGGGGGSGGGGGGSGGGGGGSGGGGGGSGGGGGGSGGGGGGSGGGGGGSGGGGGGS

SEQ ID NO: 25 - protein, 6 aa, synthetic construct
HHHHHH

SEQ ID NO: 26 - protein, 6 aa, synthetic construct
HHHHHH

SEQ ID NO: 27 - protein, 9 aa, synthetic construct
YPYDVPDYA

SEQ ID NO: 28 - protein, 10 aa, synthetic construct
EQKLISEEDL

SEQ ID NO: 29 - protein, 6 aa, synthetic construct
DTYRYI

SEQ ID NO: 30 - protein, 8 aa, synthetic construct
DYKDDDDK